(19) United States
(12) Patent Application Publication
(10) Pub. No.: US 2007/0208166 A1
(43) Pub. Date: Sep. 6, 2007

(54) Tao Kinase Modulators And Method Of Use

(75) Inventors: Deborah Lynn Baly, Lafayette, CA (US); Adam Antoni Galan, Alameda, CA (US); Mohamed Abdulkader Ibrahim, Mountain View, CA (US); Christopher Thomas Jaeger, Brisbane, CA (US); Patrick Kearney, San Francisco, CA (US); James William Leahy, San Leandro, CA (US); Gary Lee Lewis, San Francisco, CA (US); Kirk McMillan, Burlingame, CA (US); Robin Tammie Noguchi, San Bruno, CA (US); John M. Nuss, Danville, CA (US); Jason Jevious Parks, Sacramento, CA (US); Kevin Luke Schnepp, Elk Grove, CA (US); Xian Shi, San Bruno, CA (US); Matthew Alan Williams, San Mateo, CA (US); Wei Xu, Danville, CA (US); Wentao Zhang, Foster City, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Exelixis, Inc., South San Franciso, CA (US)

(21) Appl. No.: 10/576,932

(22) PCT Filed: Oct. 22, 2004
(86) PCT No.: PCT/US04/35469

§ 371 Date: Oct. 19, 2006

Related U.S. Application Data
(60) Provisional application No. 60/514,377, filed on Oct. 24, 2003.

(51) Int. Cl. C07D 471/02

(52) U.S. Cl. 534/766; 564/152; 546/336; 548/492; 546/118; 546/272.4; 548/495; 548/537; 544/250; 544/333; 544/284; 548/161; 544/278; 544/360; 540/496; 546/167; 564/50; 546/146; 548/470; 549/362; 544/386; 548/215; 548/364.1; 546/229; 548/190; 546/114

(57) ABSTRACT

The invention provides compounds and methods for inhibition of kinases, such as those of the TAO family, more specifically KIAA1361, TAO, and JIK kinases. The invention provides compounds for modulating protein kinase enzymatic activity for modulating cellular activities such as proliferation, differentiation, programmed cell death, migration and chemoinvasion. Compounds of the invention inhibit, regulate and/or modulate kinase receptor signal transduction pathways related to the changes in cellular activities as mentioned above, and the invention includes compositions which contain these compounds, and methods of using them to treat kinase-dependent diseases and conditions.

### CROSS-REFERENCE TO RELATED APPLICATIONS

This application claims priority to U.S. provisional patent applications 60/514,377 filed on Oct. 24, 2003, entitled “TAO Kinase Modulators and Methods of Use,”; which is hereby incorporated by reference in its entirety for all purposes.

### BACKGROUND OF THE INVENTION

1. Field of the Invention

This invention relates to compounds for modulating protein kinase enzymatic activity for modulating cellular activities such as proliferation, differentiation, programmed cell death, migration and chemoinvasion. Even more specifically, the invention relates to compounds which inhibit, regulate and/or modulate kinase receptor signal transduction pathways related to the changes in cellular activities as mentioned above, compositions which contain these compounds, and methods of using them to treat kinase-dependent diseases and conditions.

2. Summary of Related Art

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms.

Protein kinases are enzymes that catalyze the phosphorylation of proteins at the hydroxy groups of tyrosine, serine and threonine residues of proteins. The kinase complement of the human genome contains 518 putative protein kinase genes [Manning et al, Science, (2002), 298, 1912]. The consequences of this activity include effects on cell differentiation, proliferation, transcription, translation, metabolism, cell cycle progression, apoptosis, metabolism cytoskeletal rearrangement and movement; i.e., protein kinases mediate the majority of signal transduction in eukaryotic cells. Furthermore, abnormal protein kinase activity has been related to a host of disorders, ranging from relatively non-life threatening diseases such as psoriasis to cancer. Chromosomal mapping has revealed that over 200 kinases map to disease loci, including cancer, inflammatory and metabolic disease.

Tyrosine kinases can be categorized as receptor type or non-receptor type. Receptor-type tyrosine kinases have an extracellular, a transmembrane, and an intracellular portion, while non-receptor type tyrosine kinases are wholly intracellular.

Receptor-type tyrosine kinases are comprised of a large number of transmembrane receptors with diverse biological activity. In fact, about 20 different subfamilies of receptor-type tyrosine kinases have been identified. One tyrosine kinase subfamily, designated the HER subfamily, is comprised of EGFR (HER1), HER2, HER3, and HER4. Ligands of this subfamily of receptors identified so far include epithelial growth factor, TGF-alpha, amphiregulin, HB-EGF, betacellulin and heregulin. Another subfamily of these receptor-type tyrosine kinases is the insulin subfamily, which includes INS-R, IGF-IR, and IR-R. The PDGF subfamily includes the PDGF-alpha and beta-receptors, CSFIR, c-kit and FLK-II. Then there is the FLK family, which is comprised of the kinase insert domain receptor (KDR), fetal liver kinase-1 (FLK-1), fetal liver kinase-4 (FLK4) and the fms-like tyrosine kinase-1 (Flt-1). The PDGF and FLK families are usually considered together due to the similarities of the two groups. For a detailed discussion of the receptor-type tyrosine kinases, see Plowman et al., DN&P 7(6): 334-339, 1994, which is hereby incorporated by reference.

The non-receptor type of tyrosine kinases is also comprised of numerous subfamilies, including Src, Frk, Btk, Csk, Abl, Syk/Zap70, Fes/Fps, Fak, Jak, and Ack. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen, Oncogene, 8:2025-2031 (1993), which is hereby incorporated by reference.

Serine-threonine kinases play critical roles in intracellular signal transduction and include the multiple families, including STE, CKI, AGC, CAMK, and CMGC. Important subfamilies include, the MAP kinases, p38, JNK and ERK, which modulate signal transduction resulting from such diverse stimuli as mitogenic, stress, proinflammatory and antiapoptotic pathways. Members of the MAP kinase subfamily have been targeted for therapeutic intervention, including p38a, JNK isozymes and Raf.

Since protein kinases and their ligands play critical roles in various cellular activities, deregulation of protein kinase enzymatic activity can lead to altered cellular properties, such as uncontrolled cell growth associated with cancer. In addition to oncological indications, altered kinase signaling is implicated in numerous other pathological diseases. These include, but are not limited to: immunological disorders, cardiovascular diseases, inflammatory diseases, and degenerative diseases. Therefore, both receptor and non-receptor protein kinases are attractive targets for small molecule drug discovery.

One particularly attractive goal for therapeutic use of kinase modulation relates to oncological indications. For example, modulation of protein kinase activity for the treatment of cancer has been demonstrated successfully with the FDA approval of Gleevec® (imatinib mesylate, produced by Novartis Pharmaceutical Corporation of East Hanover, N.J.) for the treatment of Chronic Myeloid Leukemia (CML) and gastrointestinal stroma cancers. Gleevec is a selective Abl kinase inhibitor.

Modulation (particularly inhibition) of cell proliferation and angiogenesis, two key cellular processes needed for tumor growth and survival (Matter A. Drug Disc Technol 2001 6, 1005-1024), is an attractive goal for development of small-molecule drugs. Anti-angiogenic therapy represents a potentially important approach for the treatment of solid tumors and other diseases associated with dysregulated vascularization, including ischemic coronary artery disease, diabetic retinopathy, psoriasis and rheumatoid arthritis. As well, cell antiproliferative agents are desirable to slow or stop the growth of tumors.

KIAA1361, JIK (c-jun inhibiting kinase), and TAO (thousand and one amino acid kinase) are homologous serine-threonine kinases. These serine-threonine kinases are members of the STE20-related family, which participate in the modulation of the stress-activated protein MAP kinases, p38 and JNK. JIK has been reported to be decreased upon EGF receptor activation and may represent an RTK-mediated negative regulator of JNK [Tassi et al (1999) J Biol Chem 19, 274]. TAO activates p38 MAP kinase cascades by phosphorylating the MAP kinase kinases, MAP2K3 and MAP2K6 [Chen et al (2003) 278, 22278]. The role of these STE20-related family kinases in p38/JNK modulation may have therapeutic relevance to proliferative diseases, including inflammation and cancer. Furthermore, KIAA1361, JIK, and TAO may be targets for therapeutic intervention for pathologies associated with defective β-catenin signaling pathway, such as cancer [Costa et al (2003) Exelixis, Inc, USA, PCT Int. Appl. WO 2003051905].

Accordingly, the identification of small-molecule compounds that specifically inhibit, regulate and/or modulate the signal transduction of kinases, particularly at least one of KIAA1361, JIK, and TAO, more particularly KIA1361 is desirable as a means to treat or prevent disease states associated with abnormal cell proliferation, and is an object of this invention.

### SUMMARY OF THE INVENTION

In one aspect, the present invention provides compounds for modulating the activity of at least one of KIAA1361, TAO, and JIK kinases and methods of treating diseases mediated by the activity of at least one of KIAA1361, TAO, and JIK utilizing the compounds and pharmaceutical compositions thereof. Diseases mediated by KIAA1361, TAO, and JIK activity include, but are not limited to, diseases characterized in part by migration, invasion, proliferation and other biological activities associated with invasive cell growth.

In another aspect, the invention provides methods of screening for modulators of KIAA1361, TAO, and JIK activity. The methods comprise combining a composition of the invention, typically one of KIAA1361, TAO, and JIK, and at least one candidate agent and determining the effect of the candidate agent on the KIAA1361, TAO, or JIK activity.

In yet another aspect, the invention also provides pharmaceutical kits comprising one or more containers filled with one or more of the ingredients of pharmaceutical compounds and/or compositions of the present invention, including, one or more KIAA1361, TAO, or JIK enzyme activity modulators as described herein. Such kits can also include, for example, other compounds and/or compositions (e.g., diluents, permeation enhancers, lubricants, and the like), a device(s) for administering the compounds and/or compositions, and written instructions in a form prescribed by a governmental agency regulating the manufacture, use or sale of pharmaceuticals or biological products, which instructions can also reflects approval by the agency of manufacture, use or sale for human administration.

In still yet another aspect, the invention also provides a diagnostic agent comprising a compound of the invention and, optionally, pharmaceutically acceptable adjuvants and excipients.

These and other features and advantages of the present invention will be described in more detail below with reference to the associated drawings.

### DETAILED DESCRIPTION OF THE INVENTION

The compositions of the invention are used to treat diseases associated with abnormal and or unregulated cellular activities. Disease states which can be treated by the methods and compositions provided herein include, but are not limited to, cancer (further discussed below), immunological disorders such as rheumatoid arthritis, graft-host diseases, multiple sclerosis, psoriasis; cardiovascular diseases such as artheroscrosis, myocardioinfarction, ischemia, stroke and restenosis; other inflammatory and degenerative diseases such as interbowel diseases, osteoarthritus, macular degeneration, diabetic retinopathy.

It is appreciated that in some cases the cells may not be in a hyper- or hypo-proliferative and/or migratory state (abnormal state) and still require treatment. For example, during wound healing, the cells may be proliferating “normally”, but proliferation and migration enhancement may be desired. Alternatively, reduction in “normal” cell proliferation and/or migration rate may be desired.

The present invention comprises a compound for modulating kinase activity according to Formula I, 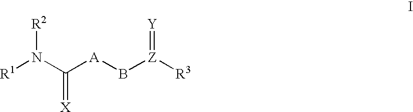
or a pharmaceutically acceptable salt, hydrate, or prodrug thereof, wherein,
R¹ is selected from optionally substituted C₁₋₁₀alkyl, optionally substituted aryl, optionally substituted aryl-C₁₋₁₀alkyl, optionally substituted heterocyclyl, and optionally substituted heterocyclyl-C₁₋₁₀alkyl;
R² is selected from —H and optionally substituted C₁₋₆alkyl;
or R¹ and R² combine to form an optionally substituted three- to seven-membered heteroalicyclic;
A is a C₁₋₃alkylene optionally substituted with one to four of R⁶;
B is selected from —O—, —N(R⁴)—, —S(O)₀₋₂— and —N(CH₂)₂N(CH₂)₂—S(O)₀₋₂—;
X is selected from ═O, ═S, and ═NR⁷;
Y is selected from ═O, ═S, and ═NR⁷;
Z is C; or
Z=Y is either absent or —CH₂—;
R³ is selected from —H, halogen, trihalomethyl, —OR⁵, —N(R⁵)R⁵, —N(R⁵)SO₂R⁵, —N(R⁵)C(O)R⁵, —NCO₂R⁵, optionally substituted alkoxy, optionally substituted alkyl, optionally substituted aryl, optionally substituted arylalkyl, optionally substituted heterocyclyl, and optionally substituted heterocyclylalkyl;
R⁴ is selected from —H and optionally substituted C₁₋₆alkyl; or
R⁴ and one of R⁶, together with the atoms to which they are attached, combine to form an optionally substituted five- to seven-membered non-aromatic ring;
each R⁵ is independently selected from —H, optionally substituted lower alkyl, optionally substituted aryl, optionally substituted lower arylalkyl, optionally substituted heterocyclyl, and optionally substituted lower heterocyclylalkyl;
two of R⁵, together with the atom or respective atoms to which they are attached, can combine to form an optionally substituted five- to seven-membered heterocyclic;
each R⁶ is independently selected from —H, halogen, trihalomethyl, —CN, —NO₂, —NH₂, —OR⁵, —NR⁵R⁵, —S(O)₀₋₂R⁵, —SO₂NR⁵R⁵, —CO₂R⁵, —C(O)NR⁵R⁵, —N(R⁵)SO₂R⁵, —N(R⁵)C(O)R⁵, —N(R⁵)CO₂R⁵, —C(O)R⁵, optionally substituted lower alkyl, optionally substituted aryl, optionally substituted heterocyclyl, optionally substituted lower heterocyclylalkyl, and optionally substituted lower arylalkyl;
two of R6, together with the atom or atoms to which they are attached, can combine to form a three to seven-membered non-aromatic ring; and
each R⁷ is independently selected from —H, —NO₂, —NH², —N(R⁵)R⁵, —CN, —OR⁵, optionally substituted lower alkyl, optionally substituted heteroalicyclylalkyl, optionally substituted aryl, optionally substituted arylalkyl and optionally substituted heteroalicyclic;
provided the compound is not one of: 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-Dimethyl-4-oxo-4H-thieno[2,3-d]pyrimidin-3-yl)-N-[(6-methoxy-benzothiazol-2-ylcarbamoyl)-methyl]-acetamide, (Naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide.

In one example, the compound is according to paragraph [0022], according to one of the following formulae: 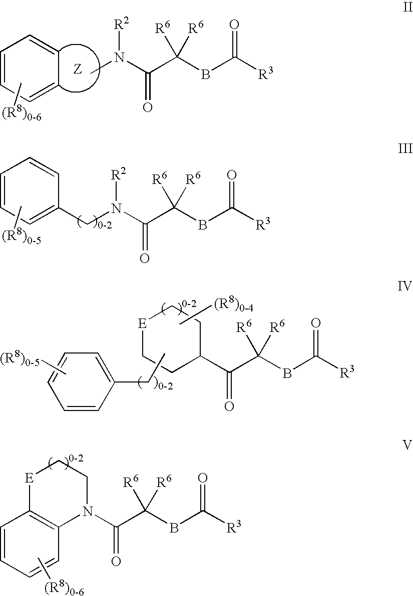
wherein R², R³, R⁶, and B are as defined above; Z is a five- to seven-membered ring; each R⁸ is independently selected from —H, halogen, trihalomethyl, —CN, —NO₂, —NH₂, —OR⁵, —NR⁵R⁵, —S(O)₀₋₂R⁵, —SO₂NR⁵R⁵, —CO₂R⁵, —C(O)NR⁵R⁵, —N(R⁵)SO₂R⁵, —N(R⁵)C(O)R⁵, —N(R⁵)CO₂R⁵, —C(O)R⁵, optionally substituted lower alkyl, optionally substituted aryl, optionally substituted heterocyclylalkyl, and optionally substituted arylalkyl; two of R⁸, together with the atom or atoms to which they are attached, can combine to form a three- to seven-membered ring; and E is selected from —O—, —N(R⁹)—, —CH₂—, and —S(O)₀₋₂—, where R⁹ is selected from —H, trihalomethyl, —S(O)₀₋₂R⁵, —SO₂NR⁵R⁵, —CO₂R⁵, —C(O)NR⁵R⁵, —C(O)R⁵, optionally substituted lower alkyl, optionally substituted aryl, optionally substituted heterocyclylalkyl, and optionally substituted arylalkyl.

In another example, the compound is according to paragraph [0022], wherein B is selected from —O—, —N(R⁴)—, and —S(O)₀₋₁—.

In another example, the compound is according to paragraph [0023], according to either formula II or III.

In another example, the compound is according to paragraph [0024], wherein R⁴ is —H or C₁₋₆alkyl.

In another example, the compound is according to paragraph [0025], wherein R² is —H or C₁₋₆alkyl.

In another example, the compound is according to paragraph [0026], according to formula IIa.

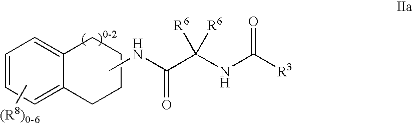

In another example, the compound is according to paragraph [0027], wherein each R⁶ is independently selected from —H, trihalomethyl, optionally substituted lower alkyl, optionally substituted aryl, optionally substituted heterocyclyl, optionally substituted heterocyclylalkyl, and optionally substituted arylalkyl.

In another example, the compound is according to paragraph [0028], wherein one of R⁶ is —H, and the other R⁶ is selected from trihalomethyl, optionally substituted lower alkyl, optionally substituted aryl, optionally substituted heterocyclyl, optionally substituted heterocyclylalkyl, and optionally substituted arylalkyl.

In another example, the compound is according to paragraph [0029], wherein R³ is selected from optionally substituted alkoxy, optionally substituted lower alkyl, optionally substituted aryl, optionally substituted lower arylalkyl, optionally substituted heterocyclyl, and optionally substituted lower heterocyclylalkyl.

In another example, the compound is according to paragraph [0030], wherein R³ is selected from lower alkyl substituted with an optionally substituted aryloxy or an optionally substituted heteroaryloxy, optionally substituted aryl, optionally substituted lower arylalkyl, optionally substituted heteroaryl, and optionally substituted lower heteroarylalkyl.

In another example, the compound is according to paragraph [0031], wherein R³ is an aryl, said aryl substituted with at least one of an optionally substituted aryl and an optionally substituted heteroaryl.

In another example, the compound is according to paragraph [0032], wherein R³ is an optionally substituted bis-phenyl.

In another example, the compound is according to paragraph [0033], wherein R³ comprises an optionally substituted phenylene, wherein the point of attachment of R³ according to formula IIa, and an optionally substituted phenyl bear a para relationship to one another about said optionally substituted phenylene.

The present invention further comprises a compound for modulating kinase activity according to formula VI, 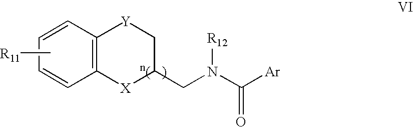
or a pharmaceutically acceptable salt, hydrate, or prodrug thereof, wherein,
each of R¹¹ and R¹² is independently selected from —H, halogen, trihalomethyl, —CN, —NO₂, —NR¹⁴R¹⁴, —S(O)₀₋₂R¹⁴, —SO₂NR¹⁴R¹⁴, —CO₂R¹⁴, —C(O)NR¹⁴R¹⁴, —N(R¹⁴)SO₂R¹⁴, —N(R¹⁴)C(O)R¹⁴, —N(R¹⁴)CO₂R¹⁴, —OR¹⁴, —C(O)R¹⁴, optionally substituted lower alkyl, optionally substituted alkoxy, optionally substituted aryl, optionally substituted heterocyclyl, optionally substituted heterocyclylalkyl, and optionally substituted arylalkyl;
R¹⁴ is selected from —H, optionally substituted lower alkyl, optionally substituted aryl, optionally substituted lower arylalkyl, optionally substituted heterocyclyl, and optionally substituted lower heterocyclylalkyl;
each of X and Y is independently selected from —O—, —N(R¹⁴)—, and —S(O)₀₋₂—;
n is selected from an integer from 0-2;
Ar is an optionally substituted aryl that may be substituted with up to three R¹¹, wherein two adjacent R¹¹'s, together with the annular atoms to which they are attached, can form a five- to seven-membered ring containing up to three heteroatoms and optionally substituted with up to three of R¹⁵;
each R¹⁵ is independently selected from —H, halo, trihalomethyl, —CN, —NO₂, —OR¹⁶, —N(R¹⁶)R¹⁶, —S(O)₀₋₂R¹⁶, —SO₂N(R¹⁶)R¹⁶, —CO₂R¹⁶, —C(═O)N(R¹⁶)R¹⁶, —C(═NR¹⁷)N(R¹⁶)R¹⁶, —C(═NR¹⁷)R¹⁶, —N(R¹⁶)SO₂R¹⁶, —N(R¹⁶)C(O)R¹⁶, —NCO₂R¹⁶, —C(═O)R¹⁶, optionally substituted alkoxy, optionally substituted lower alkyl, optionally substituted aryl, optionally substituted lower arylalkyl, optionally substituted heterocyclyl, and optionally substituted lower heterocyclylalkyl;
R¹⁶ is selected from —H, optionally substituted lower alkyl, optionally substituted aryl, optionally substituted lower arylalkyl, optionally substituted heterocyclyl, and optionally substituted lower heterocyclylalkyl; and
R¹⁷ is selected from —H, —CN, —NO₂, —OR¹⁶, —S(O)₀₋₂R¹⁶, —CO₂R¹⁶, optionally substituted lower alkyl, optionally substituted lower alkenyl, and optionally substituted lower alkynyl.

In one example, the compound is according to paragraph [0035], wherein X is O.

In another example, the compound is according to paragraph [0036], wherein Y is O.

In another example, the compound is according to paragraph [0037], wherein R¹¹ is —H.

In another example, the compound is according to paragraph [0038], wherein R¹² is —H.

In another example, the compound is according to paragraph [0039], wherein n is 1.

In another example, the compound-is according to paragraph [0040], wherein Ar is substituted aryl, wherein two adjacent R¹¹'s, together with the annular atoms to which they are attached, form a substituted six-membered ring containing up to three heteroatoms.

In another example, the compound is according to paragraph [0041], wherein Ar is substituted aryl, wherein two adjacent R¹¹'s, together with the annular atoms to which they are attached, form a substituted seven-membered ring containing up to three heteroatoms.

In another example, the compound is according to paragraphs [0021] and [0035], selected from Table 1:

TABLE 1

| Entry | Name | Structure |
|---|---|---|
| 1 | N-[2-oxo-2-(1,2,3,4- tetrahydronaphthalen-1- ylamino)ethyl]-4- (pentyloxy)benzamide | 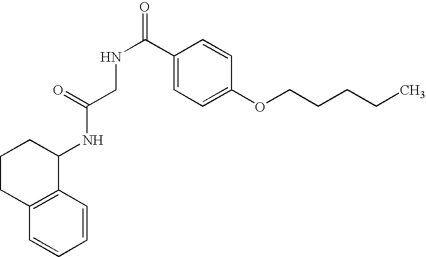 |
| 2 | N˜2˜-[({4- [(phenylmethyl)oxy]phenyl}oxy)acet yl]-N-(1,2,3,4-tetrahydronaphthalen- 1-yl)glycinamide | 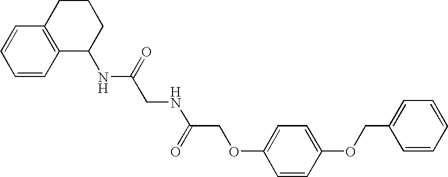 |
| 3 | N˜2˜-{[(4-bromophenyl)oxy]acetyl}- N-(1,2,3,4-tetrahydronaphthalen-1- yl)glycinamide | 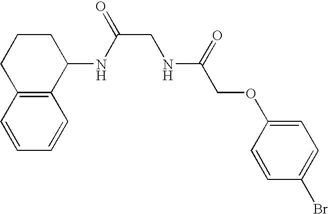 |
| 4 | 4′-ethyl-N-[2-oxo-2-(1,2,3,4- tetrahydronaphthalen-1- ylamino)ethyl]biphenyl-4- carboxamide | 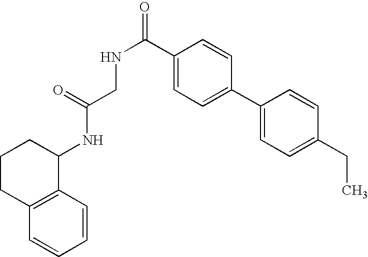 |
| 5 | 4′-ethyl-N-[1-methyl-2-oxo-2- (1,2,3,4-tetrahydronaphthalen-2- ylamino)ethyl]biphenyl-4- carboxamide | 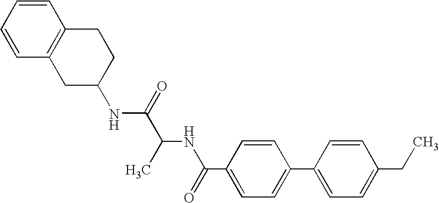 |
| 6 | 4-(hexyloxy)-N-[2-oxo-2-(1,2,3,4- tetrahydronaphthalen-1- ylamino)ethyl]benzamide | 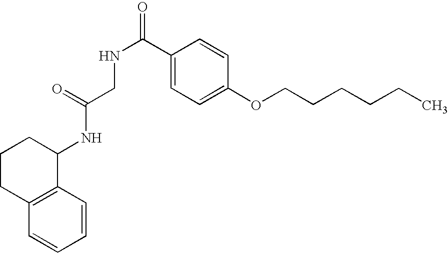 |
| 7 | 2-cyclopentyl-N-[2-oxo-1-pyridin-3- yl-2-(1,2,3,4-tetrahydronaphthalen- 1-ylamino)ethyl]-2-phenylacetamide | 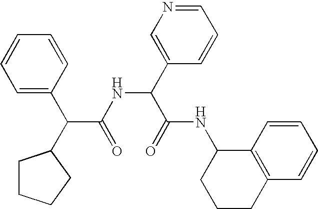 |
| 8 | 4-(heptyloxy)-N-[2-oxo-2-(1,2,3,4- tetrahydronaphthalen-1- ylamino)ethyl]benzamide | 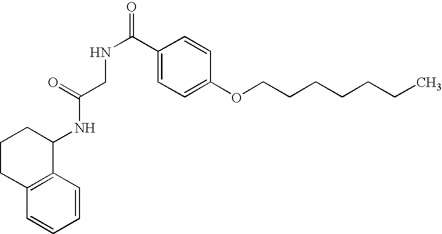 |
| 9 | N-[1-methyl-2-oxo-2-(1,2,3,4- tetrahydronaphthalen-2- ylamino)ethyl]-4- (pentyloxy)benzamide | 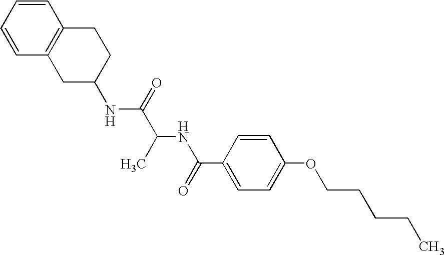 |
| 10 | 4-hexyl-N-[2-oxo-2-(1,2,3,4- tetrahydronaphthalen-1- ylamino)ethyl]benzamide | 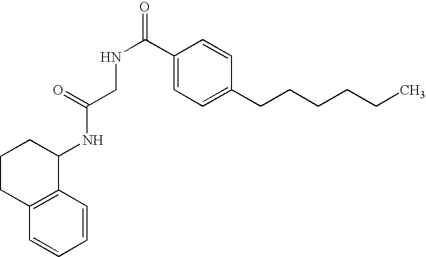 |
| 11 | N-[2-oxo-2-(1,2,3,4- tetrahydronaphthalen-1- ylamino)ethyl]-4-pentylbenzamide | 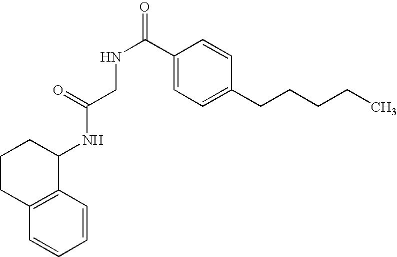 |
| 12 | 4-heptyl-N-[2-oxo-2-(1,2,3,4- tetrahydronaphthalen-1- ylamino)ethyl]benzamide | 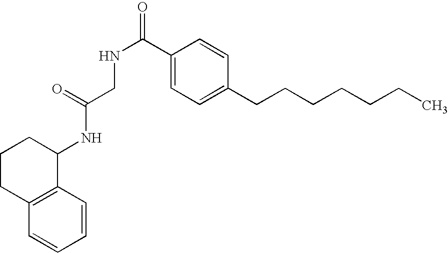 |
| 13 | Nalpha-{[5,6-bis(methyloxy)-1H- indol-2-yl]carbonyl}-N-(1,2,3,4- tetrahydronaphthalen-1- yl)tryptophanamide | 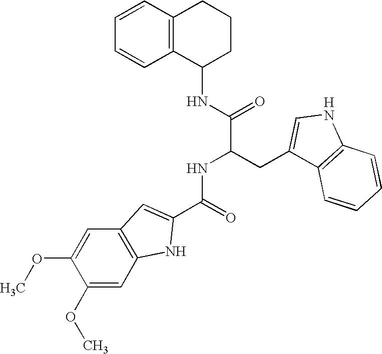 |
| 14 | Nalpha-{[4- (butyloxy)phenyl]carbonyl}-N-(1,2,3,4-tetrahydronaphthalen-1- yl)tryptophanamide | 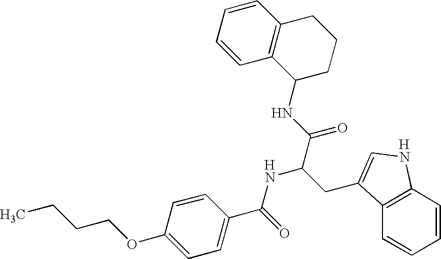 |
| 15 | 5-{(2E)-3-[3,4- bis(methyloxy)phenyl]prop-2-enoyl}- N-[2-(3-chlorophenyl)ethyl]-4,5,6,7- tetrahydro-3H-imidazol[4,5- c]pyridine-6-carboxamide | 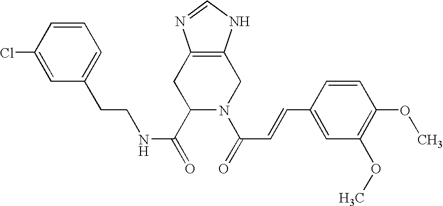 |
| 16 | N-[2-oxo-1-pyridin-3-yl-2-(1,2,3,4- tetrahydronaphthalen-1- ylamino)ethyl]-4-(1H-1,2,4-triazol-1- yl)benzamide | 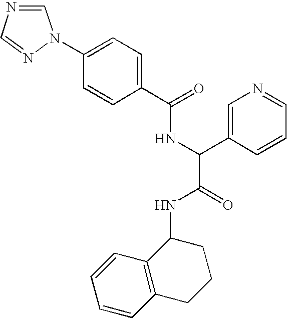 |
| 17 | Nalpha-{(2E)-3-[3,4- bis(methyloxy)phenyl]prop-2-enoyl}- N-(1,2,3,4-tetrahydronaphthalen-1- yl)tryptophanamide | 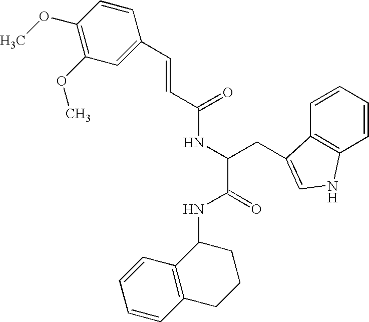 |
| 18 | 4-(butyloxy)-N-[1-methyl-2-oxo-2- (1,2,3,4-tetrahydronaphthalen-2- ylamino)ethyl]benzamide | 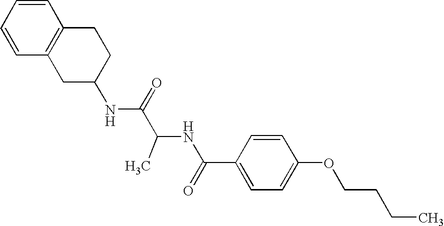 |
| 19 | N-[1-methyl-2-oxo-2-(1,2,3,4- tetrahydronaphthalen-2- ylamino)ethyl]-4- [(phenylmethyl)oxy]benzamide | 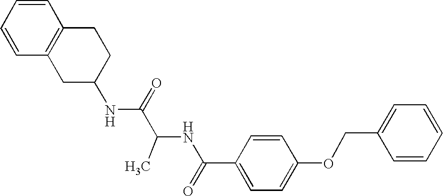 |
| 20 | 3,5-dimethyl-N-{2-oxo-2-(1,2,3,4- tetrahydronaphthalen-1- ylamino)ethyl]-4- [(phenylmethyl)oxy]benzamide | 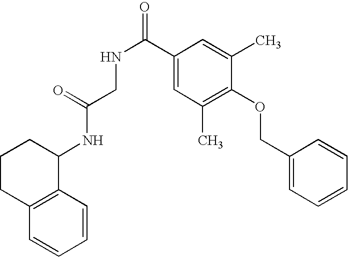 |
| 21 | N˜2˜-({[4- (butyloxy)phenyl]amino}carbonyl)- N-(2-phenylethyl)-O- (phenylmethyl)serinamide | 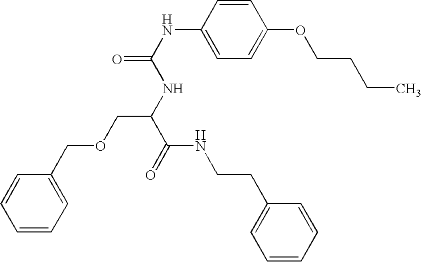 |
| 22 | (2S)-N˜-1˜-(4-butylphenyl)-N˜2˜- [(3,4- dichlorophenyl)methyl]pyrrolidine- 1,2-dicarboxamide | 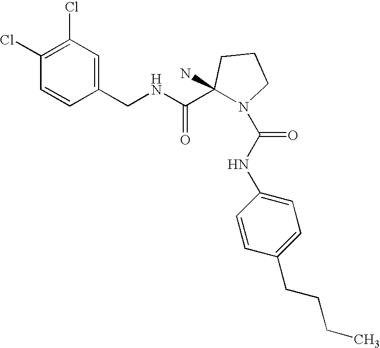 |
| 23 | N-[6-(methyloxy)-1,3-benzothiazol- 2-yl]-4-{[(4-oxo-5,6,7,8- tetrahydro[1]benzothieno[2,3- d]pyrimidin-3(4H)- yl)acetyl]amino}butanamide | 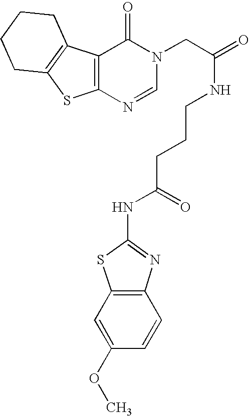 |
| 24 | (2S)-N˜2˜-[(3,4- dichlorophenyl)methyl]-N˜1˜-[4-(1- methylethyl)phenyl]pyrrolidine-1,2- dicarboxamide | 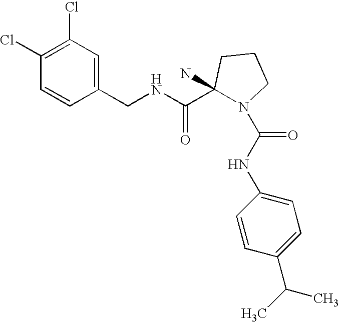 |
| 25 | N˜2˜-{[(4-bromophenyl)oxy]acetyl}- N-[(3,4- dichlorophenyl)methyl]glycinamide | 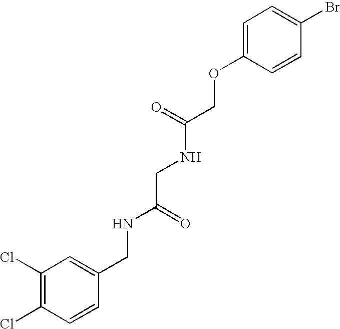 |
| 26 | N-[2-oxo-1-pyridin-3-yl-2-(1,2,3,4- tetrahydronaphthalen-1- ylamino)ethyl]-4-[(E)- phenyldiazenyl]benzamide | 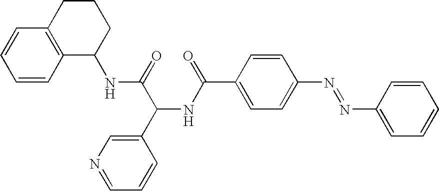 |
| 27 | 4-(butyloxy)-N-[2-oxo-2-(1,2,3,4- tetrahydronaphthalen-1- ylamino)ethyl]benzamide | 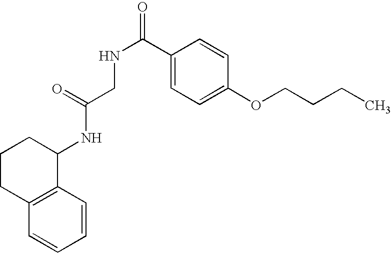 |
| 28 | N˜2˜-({[3- (methyloxy)phenyl]oxy}acetyl)-N- (1,2,3,4-tetrahydronaphthalen-1- yl)glycinamide | 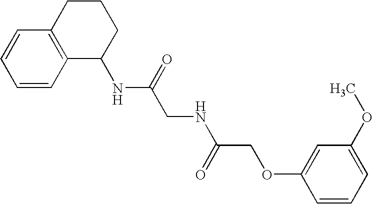 |
| 29 | 4-butyl-N-[2-oxo-2-(1,2,3,4- tetrahydronaphthalen-1- ylamino)ethyl]benzamide | 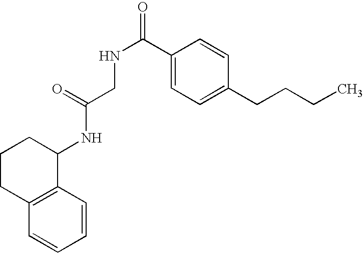 |
| 30 | N˜2˜-{(2E)-3-[3,4- bis(methyloxy)phenyl]prop-2-enoyl}- N-(diphenylmethyl)-O- (phenylmethyl)serinamide | 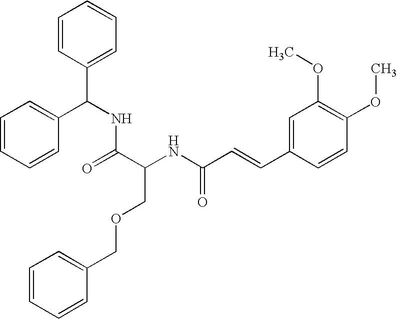 |
| 31 | N-(1,2,3,4-tetrahydronaphthalen-1- yl)-N˜2˜-[({4- [(trifluoromethyl)oxy]phenyl}oxy)ace tyl]glycinamide | 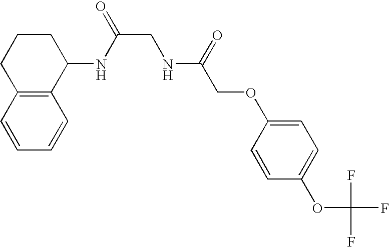 |
| 32 | N-{3-methyl-1-[(1,2,3,4- tetrahydronaphthalen-1- ylamino)carbonyl]butyl}biphenyl-4- carboxamide | 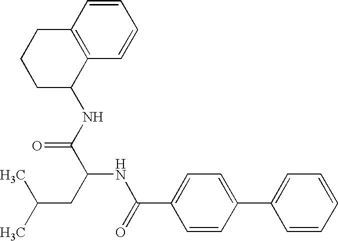 |
| 33 | N-˜2˜-({[4-(1,1- dimethylethyl)phenyl]oxy}acetyl)-N- (1,2,3,4-tetrahydronaphthalen-1- yl)glycinamide | 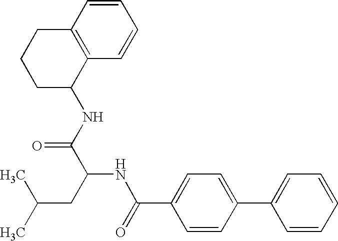 |
| 34 | N˜2˜-{[(4-chlorophenyl)oxy]acetyl}- N-(1,2,3,4-tetrahydronaphthalen-1- yl)glycinamide | 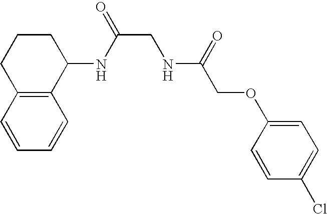 |
| 35 | N˜2˜-{[4- (phenyl]carbonyl}-N- (1,2,3,4-tetrahydronaphthalen-1- yl)leucinamide | 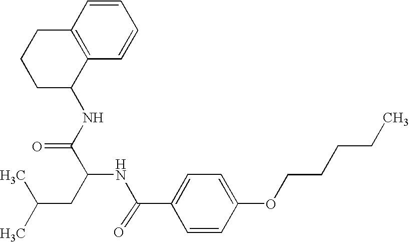 |
| 36 | 4-(hexyloxy)-N-[1-methyl-2-oxo-2- (1,2,3,4-tetrahydronaphthalen-2- ylamino)ethyl]benzamide | 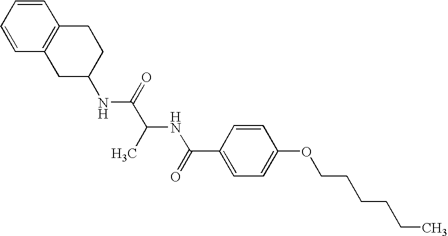 |
| 37 | 2-{[6-(methyloxy)-1,3-benzothiazol- 2-yl]amino}-2-oxoethyl 3-phenyl-3- [(phenylcarbonyl)amino]propanoate | 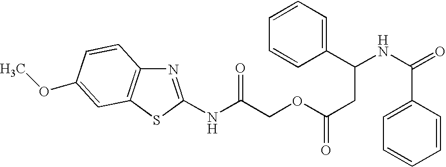 |
| 38 | N-[(3,4-dichlorophenyl)methyl]-2- {[4-(4-pyridin-2-ylpyrimidin-2- yl)phenyl]oxy]acetamide | 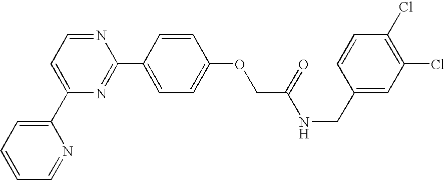 |
| 39 | N-(2,3-dihydro-1,4-benzodioxin-2- ylmethyl)-3-[3-(ethyloxy)propyl]-4- oxo-2-thioxo-1,2,3,4- tetrahydroquinazoline-7- carboxamide | 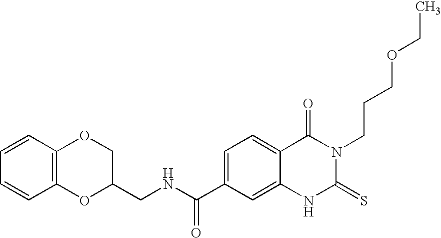 |
| 40 | N˜2˜-[(5,6-dimethyl-4- oxothieno[2,3-d]pyrimidin-3(4H)- yl)acetyl]-N-[6-(methyloxy)-1,3- benzothiazol-2-yl]glycinamide | 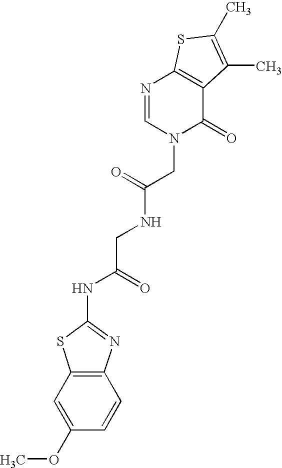 |
| 41 | 2-{4-[(2-naphthalen-1- ylethyl)sulfonyl]piperazin-1-yl)-N- pyridin-2-ylacetamide | 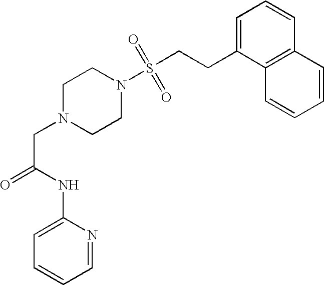 |
| 42 | N-(2,3-dihydro-1,4-benzodioxin-2- ylmethyl)-11-oxo-10,11-dihydro-5H- dibenzo[b,e][1,4]diazepine-8- carboxamide | 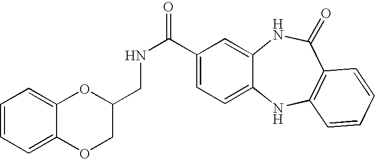 |
| 43 | N-[2-oxo-2-(1,2,3,4- tetrahydronaphthalen-1- ylamino)ethyl]biphenyl-4- carboxamide | 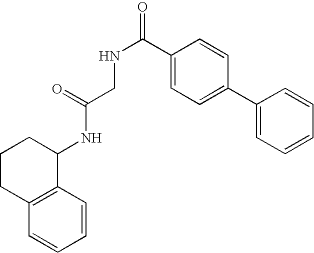 |
| 44 | N-[2-oxo-2-(1,2,3,4- tetrahydronaphthalen-1- ylamino)ethyl]naphthalene-2- carboxamide | 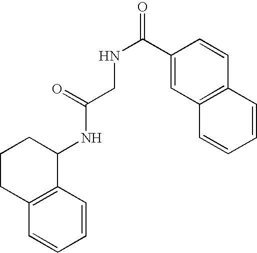 |
| 45 | N-[2-oxo-2-(1,2,3,4- tetrahydronaphthalen-1- ylamino)ethyl]-4- [(trifluoromethyl)oxy]benzamide | 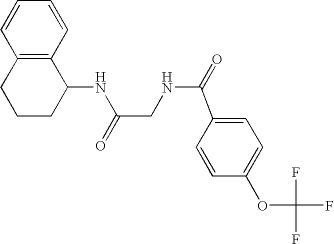 |
| 46 | N-[2-oxo-2-(1,2,3,4- tetrahydronaphthalen-1- ylamino)ethyl]quinoline-3- carboxamide | 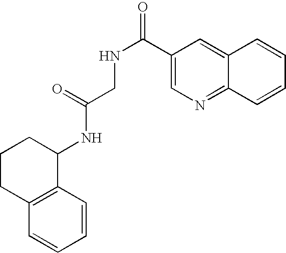 |
| 47 | N˜2˜-({[3,5- bis(trifluoromethyl)phenyl]amino}car bonyl)-N-(1,2,3,4- tetrahydronaphthalen-1- yl)glycinamide | 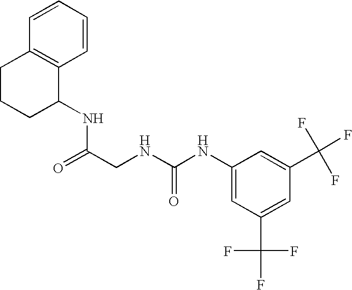 |
| 48 | N˜2˜-{[(3- ethylphenyl)amino]carbonyl}-N- (1,2,3,4-tetrahydronaphthalen-1- yl)glycinamide | 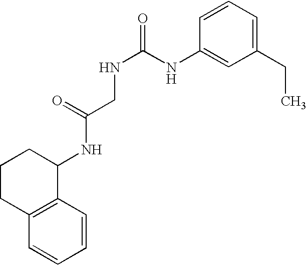 |
| 49 | N-[2-oxo-2-(1,2,3,4- tetrahydronaphthalen-1- ylamino)ethyl]-4- (phenyloxy)benzamide | 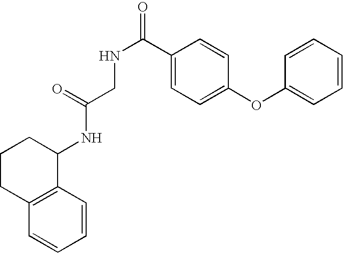 |
| 50 | 4-cyclohexyl-N-[2-oxo-2-(1,2,3,4- tetrahydronaphthalen-1- ylamino)ethyl]benzamide | 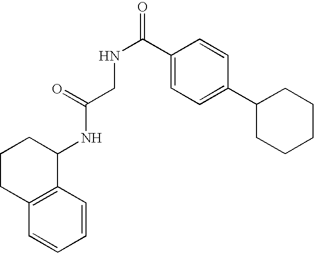 |
| 51 | N-[2-oxo-2-(1,2,3,4- tetrahydronaphthalen-1- ylamino)ethyl]-4- [(phenylmethyl)oxy]benzamide | 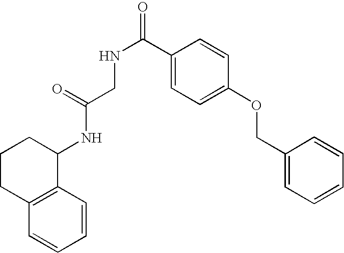 |
| 52 | 4-ethyl-N-{2-oxo-2- [(phenylmethyl)amino]ethyl}biphenyl -4-carboxamide | 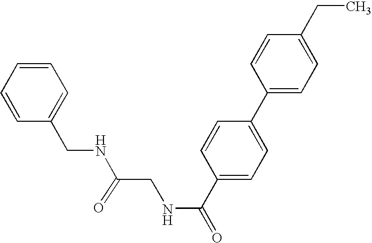 |
| 53 | N-[2-(cyclohexylamino)-2-oxoethyl]- 4′-ethylbiphenyl-4-carboxamide | 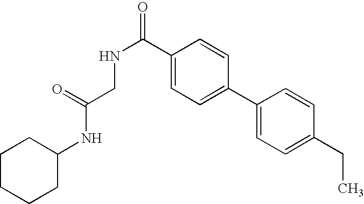 |
| 54 | N-(2-{[(3,4- dichlorophenyl)methyl]amino}-2- oxoethyl)-4′-ethylbiphenyl-4- carboxamide | 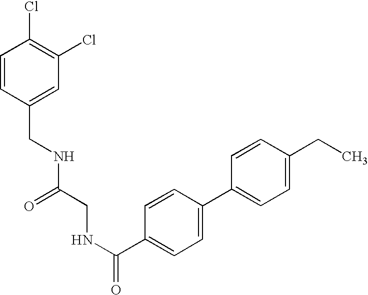 |
| 55 | N˜2˜- [{[cyclopentyl(phenyl)acetyl]amino}(pyridin-3-yl)acetyt]-N-(1,2,3,4- tetrahydronaphthalen-1- yl)glycinamide | 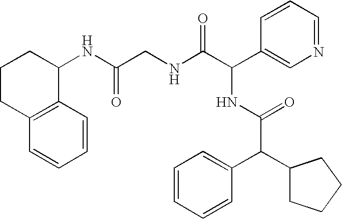 |
| 56 | 4-hydroxy-N-[2-oxo-2-(1,2,3,4- tetrahydronaphthalen-1- ylamino)ethyl]benzamide | 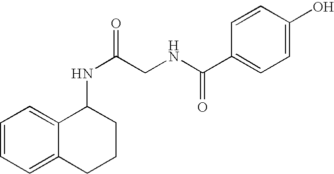 |
| 57 | N-[2-(1,3-dihydro-2H-isoindol-2-yl)- 2-oxoethyl]-4′-ethylbiphenyl-4- carboxamide | 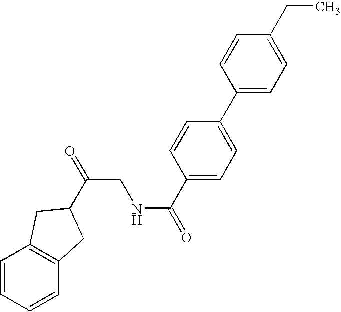 |
| 58 | 4-morpholin-4-yl-N-[2-oxo-2- (1,2,3,4-tetrahydronaphthalen-1- ylamino)ethyl]benzamide | 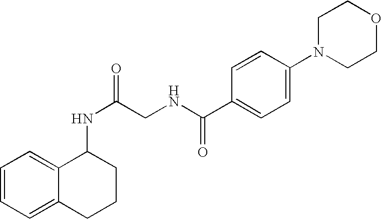 |
| 59 | 5,6-bis(methyloxy)-N-(2-oxo-2- (1,2,3,4-tetrahydronaphthalen-1- ylamino)ethyl]-1H-indole-2- carboxamide | 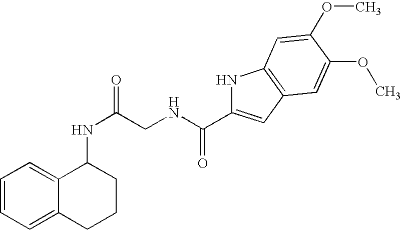 |
| 60 | N-[2-(3,4-dihydroisoquinolin-2(1H)- yl)-2-oxoethyl]-4′-ethylbiphenyl-4- carboxamide | 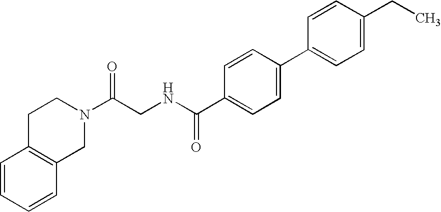 |
| 61 | 4′-ethyl-N-{2-oxo-2-[(1S)-1,2,3,4- tetrahydronaphthalen-1- ylamino]ethyl}biphenyl-4- carboxamide | 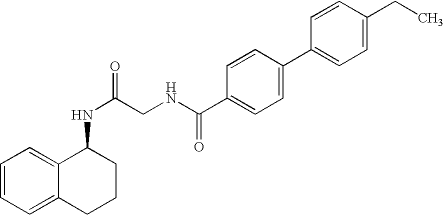 |
| 62 | 2-amino-N-˜4˜-(2,3-dihydro-1,4- benzodioxin-2-ylmethyl)-N˜1˜-[3- (ethyloxy)propyl]benzene-1,4- dicarboxamide | 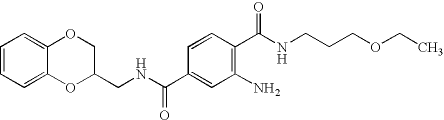 |
| 63 | N-{3-[(2-{[6-(methyloxy)-1,3- benzothiazol-2-yl]amino}-2- oxoethyl)amino]-3-oxo-1- phenylpropyl}benzamide | 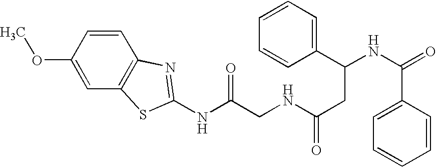 |
| 64 | 4′ethyl-N-(2-{[6-(methyloxy)-1,3- benzothiazol-2-yl]amino}-2- oxoethyl)biphenyl-4-carboxamide | 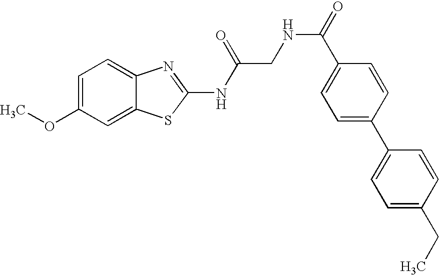 |
| 65 | N-{2-[(2,3-dihydro-1,4-benzodioxin- 2-ylmethyl)amino]-2-oxoethyl}-4′- ethylbiphenyl-4-carboxamide | 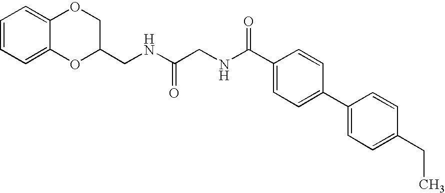 |
| 66 | 4′-ethyl-N-methyl-N-[2-oxo-2- (1,2,3,4-tetrahydronaphthalen-1- ylamino)ethyl]biphenyl-4- carboxamide | 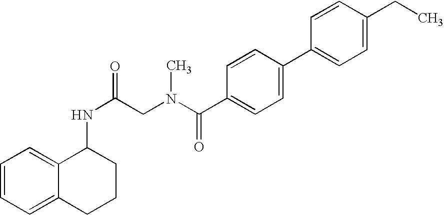 |
| 67 | 4′-ethyl-N-[2-(naphthalen-1- ylamino)-2-oxoethyl]biphenyl-4- carboxamide | 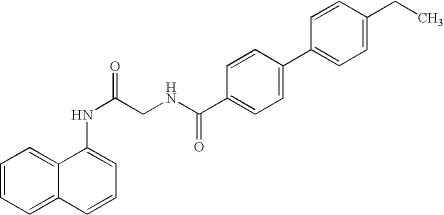 |
| 68 | 4′-ethyl-N-[2-oxo-2-(4- phenylpiperazin-1-yl)ethyl]biphenyl- 4-carboxamide | 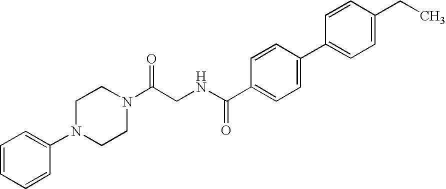 |
| 69 | N˜2˜-(biphenyl-4-ylmethyl)-N- (1,2,3,4-tetrahydronaphthalen-1- yl)glycinamide | 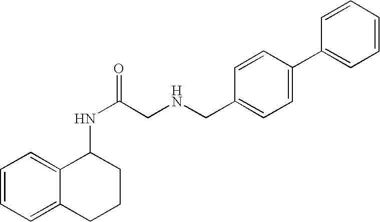 |
| 70 | 4-(1H-imidazol-1-yl)-N-[2-oxo-2- (1,2,3,4-tetrahydronaphthalen-1- ylamino)ethyl]benzamide | 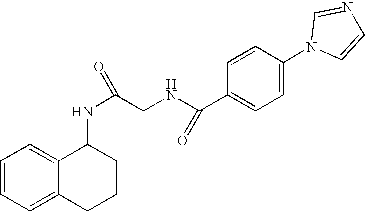 |
| 71 | 4-(1,3-oxazol-5-yl)-N-[2-oxo-2- (1,2,3,4-tetrahydronaphthalen-1- ylamino)ethyl]benzamide | 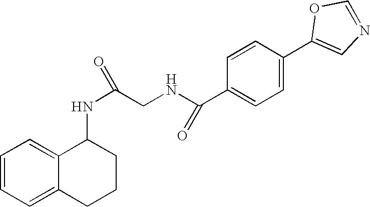 |
| 72 | N˜2˜-{[(2-biphenyl-4- ylethyl)amino]carbonyl}-N-(1,2,3,4- tetrahydronaphthalen-1- yl)glycinamide | 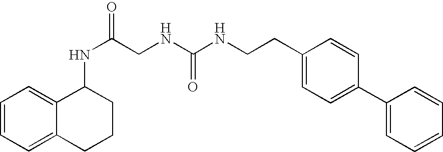 |
| 73 | N˜2˜-[(biphenyl-4- ylamino)carbonyl]-N-(1,2,3,4- tetrahydronaphthalen-1- yl)glycinamide | 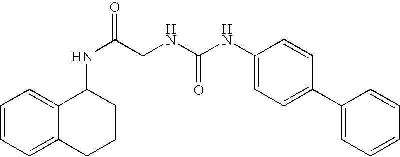 |
| 74 | 4′-ethyl-N-(2-{[(2- methylphenyl)methyl]amino}-2- oxoethyl)biphenyl-4-carboxamide | 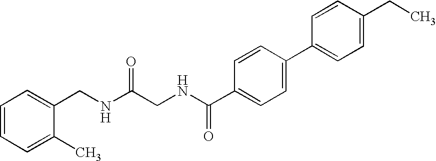 |
| 75 | 4′-ethyl-N-{2-oxo-2-[(2- phenylethyl)amino]ethyl}biphenyl-4- carboxamide | 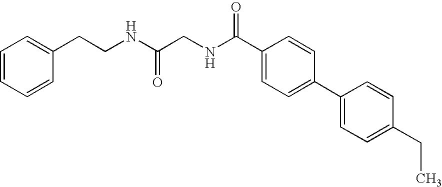 |
| 76 | 4′-ethyl-N-{2-oxo-2-[(1R)-1,2,3,4- tetrahydronaphthalen-1- ylamino]ethyl}biphenyl-4- carboxamide | 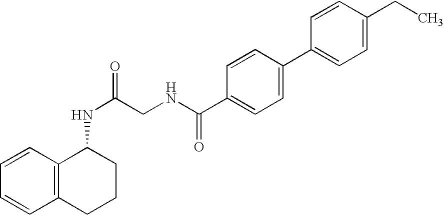 |
| 77 | N-[2-oxo-2-(1,2,3,4- tetrahydronaphthalen-1- ylamino)ethyl]-4-piperidin-1- ylbenzamide | 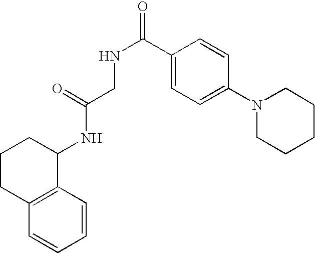 |
| 78 | 4′-ethyl-N-{2-oxo-2-[(1- phenylethyl)amino]ethyl}biphenyl-4- carboxamide | 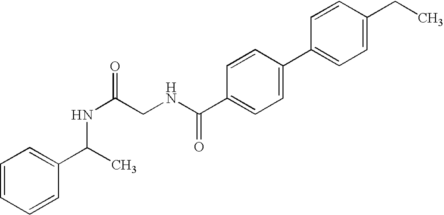 |
| 79 | 4′-ethyl-N-[2-oxo-2-({[2- (trifluoromethyl)phenyl]methyl}amino) ethyl]biphenyl-4-carboxamide | 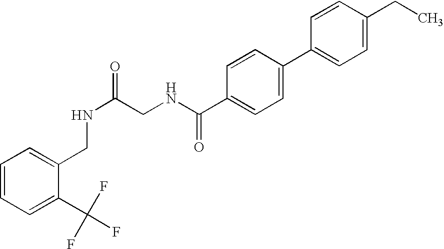 |
| 80 | N-[2-oxo-2-(1,2,3,4- tetrahydronaphthalen-1- ylamino)ethyl]quinoline-6- carboxamide | 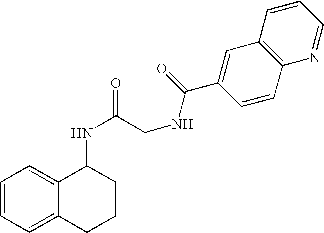 |
| 81 | N-(3-oxo-3-{[2-oxo-2-(1,2,3,4- tetrahydronaphthalen-1- ylamino)ethyl]amino}-1- phenylpropyl)benzamide | 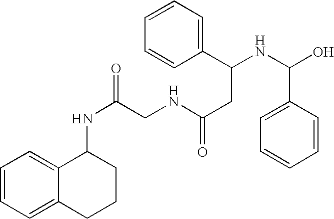 |
| 82 | N-[2-oxo-2-(1,2,3,4- tetrahydronaphthalen-1- ylamino)ethyl]-5,6,7,8- tetrahydronaphthalene-2- carboxamide | 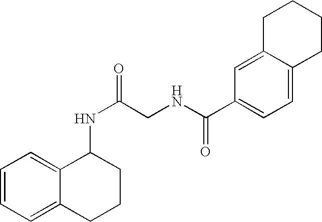 |
| 83 | 4′-ethyl-N-{2-oxo-2-[(1- phenylpropyl)amino]ethyl}biphenyl- 4-carboxamide | 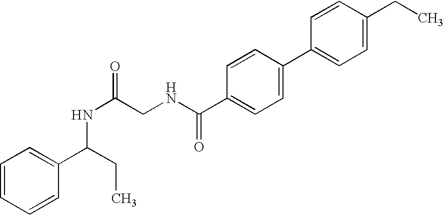 |
| 84 | 3-(acetylamino)-N-(2-{[6- (methyloxy)-1,3-benzothiazol-2- yl]amino}-2-oxoethyl)-3- phenylpropanamide | 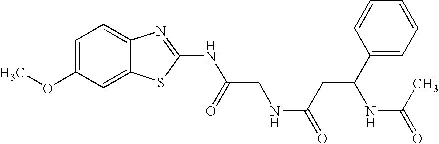 |
| 85 | N-(phenylcarbonyl)-beta-alanyl-N- [6-(methyloxy)-1,3-benzothiazol-2- yl]glycinamide | 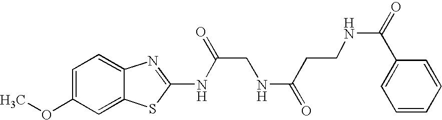 |
| 86 | N-[2-oxo-2-(5,6,7,8- tetrahydronaphthalen-1- ylamino)ethyl]quinoline-6- carboxamide | 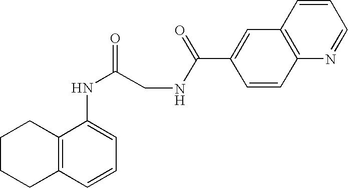 |
| 87 | N-[2-oxo-2-(5,6,7,8- tetrahydronaphthalen-1- ylamino)ethyl]quinoline-3- carboxamide | 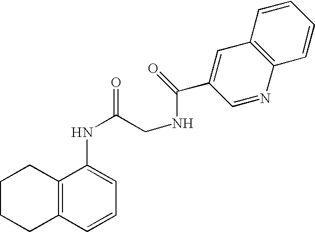 |
| 88 | N-[2-oxo-2-(1,2,3,4- tetrahydronaphthalen-1- ylamino)ethyl]-3-piperidin-1- ylpropanamide | 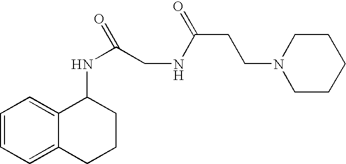 |
| 89 | N-[2-oxo-2-(5,6,7,8- tetrahydronaphthalen-1- ylamino)ethyl]-4-piperidin-1- ylbenzamide | 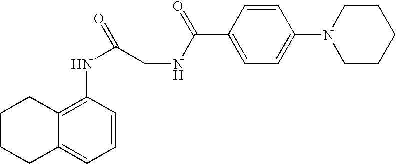 |
| 90 | N-(3-{[2-(1,3-benzothiazol-2- ylamino)-2-oxoethyl]amino}-3-oxo- 1-phenylpropyl)benzamide | 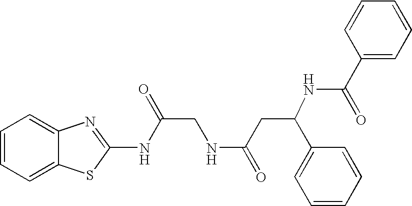 |
| 91 | N-[2-oxo-2-(1,2,3,4- tetrahydronaphthalen-1- ylamino)ethyl]-4-phenylpiperazine 1-carboxamide | 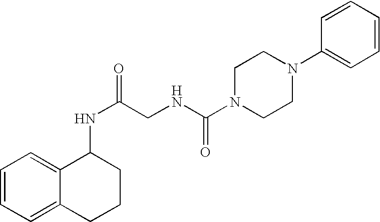 |
| 92 | N-(3-oxo-3-{[2-oxo-2-(1,3-thiazol-2- ylamino)ethyl]amino}-1- phenylpropyl)benzamide | 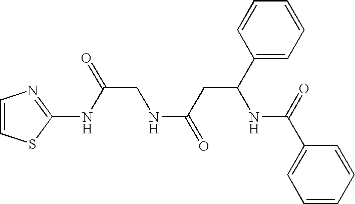 |
| 93 | N-[3-({2-[(4-methyl-1,3-thiazol-2- yl)amino]-2-oxoethyl}amino)-3-oxo- 1-phenylpropyl]benzamide | 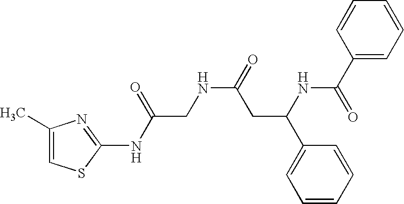 |
| 94 | N-{3-[(2-{[5- (methyloxy)[1,3]thiazolo[5,4- b]pyridin-2-yl]amino}-2- oxoethyl)amino]-3-oxo-1- phenylpropyl}benzamide | 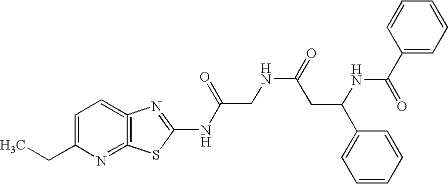 |
| 95 | 1,1-dimethylethyl 4-({N-[(4′- ethylbiphenyl-4- yl)carbonyl]glycyl}amino)-3,4- dihydroisoquinoline-2(1H)- carboxylate | 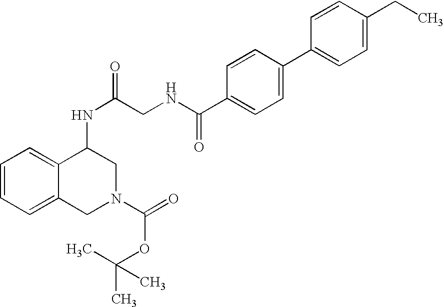 |
| 96 | 4′-ethyl-N-[2-oxo-2-(1,2,3,4- tetrahydroisoquinolin-4- ylamino)ethyl]biphenyl-4- carboxamide | 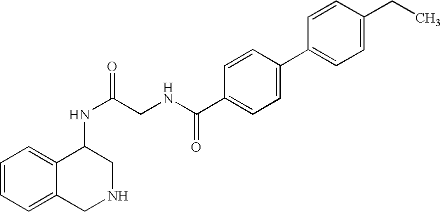 |
| 97 | N-{3-[(6-hydroxy-1,3-benzothiazol- 2-yl)amino]-3-oxo-1- phenylpropyl)benzamide | 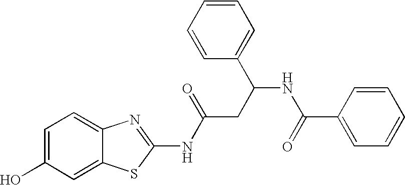 |
| 98 | N-[3-({2-[(6-hydroxy-1,3- benzothiazol-2-yl)amino]-2- oxoethyl}amino)-3-oxo-1- phenylpropyl]benzamide | 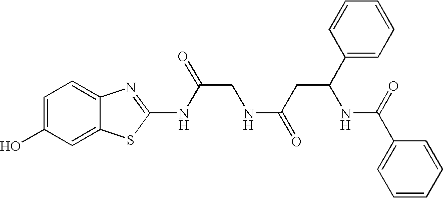 |
| 99 | 2-[(5-bromopyridin-2-yl)amino]-2- oxoethyl{[2-(naphthalen-1- ylamino)-2-oxoethyl]thio}acetate | 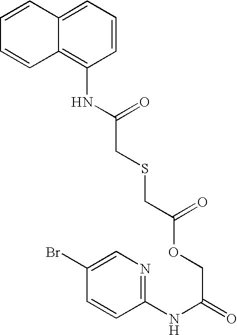 |
| 100 | 2-[(5-chloropyridin-2-yl)amino]-2- oxoethyl(1,3-benzoxazol-2- ylthio)acetate | 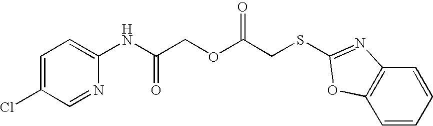 |
| 101 | 4-{[(5,6-dimethyl-4-oxothieno[2,3- d]pyrimidin-3(4H)-yl)acetyl]amino}- N-[6-(methyloxy)-1,3-benzothiazol- 2-yl]butanamide | 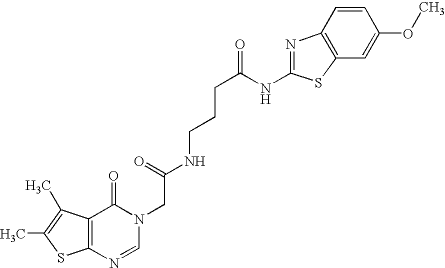 |

Another aspect of the invention is a pharmaceutical composition comprising a compound according to any one of paragraphs [0021]-[0035] and a pharmaceutically acceptable carrier.

Another aspect of the invention is a metabolite of the compound or the pharmaceutical composition according to any one of paragraphs [0021]-[0045].

Another aspect of the invention is a method of modulating the in vivo activity of a kinase, the method comprising administering to a subject an effective amount of either the compound or the pharmaceutical composition according to any of paragraphs [0021]-[0045], or a compound, or pharmaceutical composition comprising said compound and a pharmaceutically acceptable carrier, selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-dimethyl-4-oxo-4H-thieno[2,3-d]pyrimidin-3-yl)-N-[(6-methoxy-benzothiazol-2-ylcarbamoyl)-methyl]-acetamide, (naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide.

Another aspect of the invention is the method according to paragraph [0047], wherein the kinase is at least one of KIAA1361, TAO, and JIK.

Another aspect of the invention is the method according to paragraph [0048], wherein modulating the in vivo activity of the kinase comprises inhibition of said kinase.

Another aspect of the invention is a method of treating diseases or disorders associated with uncontrolled, abnormal, and/or unwanted cellular activities, the method comprising administering, to a mammal in need thereof, a therapeutically effective amount of either the compound or the pharmaceutical composition as described in any one of paragraphs [0021]-[0045], or a compound, or pharmaceutical composition comprising said compound and a pharmaceutically acceptable carrier, selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-Dimethyl-4-oxo-4H-thieno[2,3-d]pyrimidin-3-yl)-N-[(6-methoxy-benzothiazol-2-ylcarbamoyl)-methyl]-acetamide, (Naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide.

Another aspect of the invention is a method of screening for a modulator of a kinase, said kinase selected from KIAA1361, TAO, and JIK, the method comprising combining either a compound according to any one of paragraphs [0021]-[0044], or a compound selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-dimethyl-4-oxo-4H-thieno[2,3-d]pyrimidin-3-yl)-N-[(6-methoxy-benzothiazol-2-ylcarbamoyl)-methyl]-acetamide, (naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide, and at least one candidate agent and determining the effect of the candidate agent on the activity of said kinase.

Another aspect of the invention is a method of inhibiting proliferative activity in a cell, the method comprising administering an effective amount of a composition comprising a compound according any one of paragraphs [0021]-[0044], or a compound selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-dimethyl-4-oxo-4H-thieno[2,3-d]pyrimidin-3-yl)-N-[(6-methoxy-benzothiazol-2-ylcarbamoyl)-methyl]-acetamide, (naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide, to a cell or a plurality of cells.

Definitions

As used in the present specification, the following words and phrases are generally intended to have the meanings as set forth below, except to the extent that the context in which they are used indicates otherwise or they are expressly defined to mean something different.

The symbol “—” means a single bond, “═” means a double bond, “≡” means a triple bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent formula, the “ [symbol] ” symbol will be used at the end of the bond which was theoretically cleaved in order to separate the group from its parent structural formula.

If a group “R” is depicted as “floating” on a ring system, as for example in the formula: 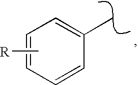
then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae: 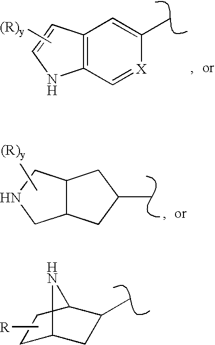
then, unless otherwise defined, a substituent “R” may reside on any atom of the fused ring system, assuming replacement of a depicted (for example the —NH— in the formula above), implied (for example as in the formula above, where the hydrogens are not shown but understood to be present), or expressly defined hydrogen (for example where in the formula above, “X” equals —CH—) from one of the ring atoms, so long as a stable structure is formed. In the example depicted, the “R” group may reside on either the 5-membered or the 6-membered ring of the fused ring system. In the formula depicted above, when y is 2 for example, then the two “R's” may reside on any two atoms of the ring system, again assuming each replaces a depicted, implied, or expressly defined hydrogen on the ring.

When there are more than one such depicted “floating” groups, as for example in the formulae: 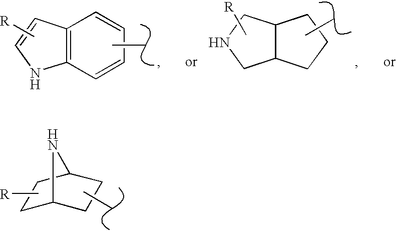
where there are two groups, namely, the “R” and the bond indicating attachment to a parent structure; then, unless otherwise defined, the “floating” groups may reside on any atoms of the ring system, again assuming each replaces a depicted, implied, or expressly defined hydrogen on the ring.

When a group “R” is depicted as existing on a ring system containing saturated carbons, as for example in the formula: 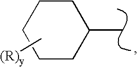
where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

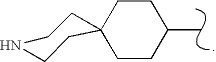

“Alkyl” is intended to include linear, branched, or cyclic hydrocarbon structures and combinations thereof, inclusively. For example, “C₈ alkyl” may refer to an n-octyl, iso-octyl, cyclohexylethyl, and the like. Lower alkyl refers to alkyl groups of from one to six carbon atoms. Examples of lower alkyl groups include methyl, ethyl, propyl, isopropyl, butyl, s-butyl, t-butyl, isobutyl, pentyl, hexyl and the like. Higher alkyl refers to alkyl groups containing more that eight carbon atoms. Exemplary alkyl groups are those of C₂₀ or below. Cycloalkyl is a subset of alkyl and includes cyclic hydrocarbon groups of from three to thirteen carbon atoms. Examples of cycloalkyl groups include c-propyl, c-butyl, c-pentyl, norbornyl, adamantyl and the like. In this application, alkyl refers to alkanyl, alkenyl, and alkynyl residues (and combinations thereof); it is intended to include cyclohexylmethyl, vinyl, allyl, isoprenyl, and the like. Thus when an alkyl residue having a specific number of carbons is named, all geometric isomers having that number of carbons are intended to be encompassed; thus, for example, either “butyl” or “C₄ alkyl” is meant to include n-butyl, sec-butyl, isobutyl, t-butyl, isobutenyl and but-2-yne radicals; and for example, “propyl” or “C₃ alkyl” each include n-propyl, propenyl, and isopropyl.

“Alkylene” refers to straight or branched chain divalent radical consisting solely of carbon and hydrogen atoms, containing no unsaturation and having from one to ten carbon atoms, for example, methylene, ethylene, propylene, n-butylene and the like. Alkylene is a subset of alkyl, referring to the same residues as alkyl, but having two points of attachment and, specifically, fully saturated. Examples of alkylene include ethylene (—CH₂CH₂—), propylene (—CH₂CH₂CH₂—), dimethylpropylene (—CH₂C(CH₃)₂CH₂—), and cyclohexylpropylene (—CH₂CH₂CH(C₆H₁₃)).

“Alkylidene” refers to a straight or branched chain unsaturated divalent radical consisting solely of carbon and hydrogen atoms, having from two to ten carbon atoms, for example, ethylidene, propylidene, n-butylidene, and the like. Alkylidene is a subset of alkyl, referring to the same residues as alkyl, but having two points of attachment and, specifically, double bond unsaturation. The unsaturation present includes at least one double bond.

“Alkylidyne” refers to a straight or branched chain unsaturated divalent radical consisting solely of carbon and hydrogen atoms having from two to ten carbon atoms, for example, propylid-2-ynyl, n-butylid-1-ynyl, and the like. Alkylidyne is a subset of alkyl, referring to the same residues as alkyl, but having two points of attachment and, specifically, triple bond unsaturation. The unsaturation present includes at least one triple bond.

Any of the above radicals, “alkylene,” “alkylidene” and “alkylidyne,” when optionally substituted, may contain alkyl substitution which itself contains unsaturation. For example, 2-(2-phenylethynyl-but-3-enyl)-naphthalene (IUPAC name) contains an n-butylid-3-ynyl radical with a vinyl substituent at the 2-position of said radical.

“Alkoxy” or “alkoxyl” refers to the group —O-alkyl, for example including from one to eight carbon atoms of a straight, branched, cyclic configuration, unsaturated chains, and combinations thereof attached to the parent structure through an oxygen atom. Examples include methoxy, ethoxy, propoxy, isopropoxy, cyclopropyloxy, cyclohexyloxy and the like. Lower-alkoxy refers to groups containing one to six carbons.

“Substituted alkoxy” refers to the group —O-(substituted alkyl), the substitution on the alkyl group generally containing more than only carbon (as defined by alkoxy). One exemplary substituted alkoxy group is “polyalkoxy” or —O-optionally substituted alkylene-optionally substituted alkoxy, and includes groups such as —OCH₂CH₂OCH₃, and glycol ethers such as polyethyleneglycol and —O(CH₂CH₂O)ₓCH₃, where x is an integer of between about two and about twenty, in another example, between about two and about ten, and in a further example between about two and about five. Another exemplary substituted alkoxy group is hydroxyalkoxy or —OCH₂(CH₂)_{y}OH, where y is for example an integer of between about one and about ten, in another example y is an integer of between about one and about four. Yet another exemplary substituted alkoxy group is arylalkoxy or heteroarylalkoxy, in these groups the alkyl portion of the alkoxy is substituted with an aryl or heteroaryl, the aryl or heteroaryl is preferably optionally substituted.

“Acyl” refers to groups of from one to ten carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to six carbons.

“α-Amino Acids” refer to naturally occurring and commercially available amino acids and optical isomers thereof. Typical natural and commercially available α-amino acids are glycine, alanine, serine, homoserine, threonine, valine, norvaline, leucine, isoleucine, norleucine, aspartic acid, glutamic acid, lysine, omithine, histidine, arginine, cysteine, homocysteine, methionine, phenylalanine, homophenylalanine, phenylglycine, ortho-tyrosine, meta-tyrosine, para-tyrosine, tryptophan, glutamine, asparagine, proline and hydroxyproline. A “side chain of an α-amino acid” refers to the radical found on the α-carbon of an α-amino acid as defined above, for example, hydrogen (for glycine), methyl (for alanine), benzyl (for phenylalanine), and the like.

“Amino” refers to the group —NH₂. “Substituted amino,” refers to the group —N(H)R or —N(R)R where each R is independently selected from the group: optionally substituted alkyl, optionally substituted alkoxy, optionally substituted aryl, optionally substituted heterocyclyl, acyl, carboxy, alkoxycarbonyl, sulfanyl, sulfinyl and sulfonyl, for example, diethylamino, methylsulfonylamino, furanyl-oxy-sulfonamino.

“Aryl” refers to aromatic six- to fourteen-membered carbocyclic ring, for example, benzene, naphthalene, indane, tetralin, fluorene and the like, univalent radicals. As univalent radicals, the aforementioned ring examples are named, phenyl, naphthyl, indanyl, tetralinyl, and fluorenyl.

“Arylene” generically refers to any aryl that has at least two groups attached thereto. For a more specific example, “phenylene” refers to a divalent phenyl ring radical. A phenylene, thus may have more than two groups attached, but is defined by the minimum of two groups attached thereto.

“Arylalkyl” refers to a residue in which an aryl moiety is attached to a parent structure via one of an alkylene, alkylidene, or alkylidyne radical. Examples include benzyl, phenethyl, phenylvinyl, phenylallyl and the like. Both the aryl, and the corresponding alkylene, alkylidene, or alkylidyne radical portion of an arylalkyl group may be optionally substituted. “Lower arylalkyl” refers to an arylalkyl where the “alkyl” portion of the group has one to six carbons; this can also be referred to as C₁₋₆ arylalkyl.

“Aryloxy” refers to a residue in which an aryl moiety is attached to a parent structure via an intervening oxygen, depicted in formula as —O—. Examples include phenyloxy, and naphthyloxy. “Substituted aryloxy” refers to an aryloxy, where the aryl portion of the aryloxy is substituted with the group or groups identified as substituents.

“Exo-alkenyl” refers to a double bond that emanates from an annular carbon, and is not within the ring system, for example the double bond depicted in the formula below.

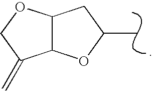

In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Fused-polycyclic” or “fused ring system” refers to a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the invention may themselves have spiro rings attached thereto via a single ring atom of the fused-polycyclic.

“Halogen” or “halo” refers to fluorine, chlorine, bromine or iodine. “Haloalkyl” and “haloaryl” refer generically to alkyl and aryl radicals that are substituted with one or more halogens, respectively. Thus, “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is within the scope of dihaloaryl.

“Heteroatom” refers to O, S, N, or P.

“Heterocyclyl” refers to a stable three- to fifteen-membered ring radical that consists of carbon atoms and from one to five heteroatoms selected from the group consisting of nitrogen, phosphorus, oxygen and sulfur. For purposes of this invention, the heterocyclyl radical may be a monocyclic, bicyclic or tricyclic ring system, which may include fused or bridged ring systems as well as spirocyclic systems; and the nitrogen, phosphorus, carbon or sulfur atoms in the heterocyclyl radical may be optionally oxidized to various oxidation states. In a specific example, the group —S(O)₀₋₂—, refers to —S— (sulfide), —S(O)— (sulfoxide), and —SO₂— (sulfone). For convenience, nitrogens, particularly but not exclusively, those defined as annular aromatic nitrogens, are meant to include their corresponding N-oxide form, although not explicitly defined as such in a particular example. Thus, for a compound of the invention having, for example, a pyridyl ring; the corresponding pyridyl-N-oxide is meant to be included as another compound of the invention. In addition, annular nitrogen atoms may be optionally quaternized; and the ring radical may be partially or fully saturated or aromatic. Examples of heterocyclyl radicals include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Heteroalicyclic” refers specifically to a non-aromatic heterocyclyl radical. A heteroalicyclic may contain unsaturation, but is not aromatic.

“Heteroaryl” refers specifically to an aromatic heterocyclyl radical.

“Heterocyclylalkyl” refers to a residue in which a heterocyclyl is attached to a parent structure via one of an alkylene, alkylidene, or alkylidyne radical. Examples include (4-methylpiperazin-1-yl) methyl, (morpholin-4-yl) methyl, (pyridine-4-yl) methyl, 2-(oxazolin-2-yl) ethyl, 4-(4-methylpiperazin-1-yl)-2-butenyl, and the like. Both the heterocyclyl, and the corresponding alkylene, alkylidene, or alkylidyne radical portion of a heterocyclylalkyl group may be optionally substituted. “Lower heterocyclylalkyl” refers to a heterocyclylalkyl where the “alkyl” portion of the group has one to six carbons. “Heteroalicyclylalkyl” refers specifically to a heterocyclylalkyl where the heterocyclyl portion of the group is non-aromatic; and “heteroarylalkyl” refers specifically to a heterocyclylalkyl where the heterocyclyl portion of the group is aromatic.

“Heteroaryloxy” refers to a residue in which a heteroaryl moiety is attached to a parent structure via an intervening oxygen, depicted in formula as —O—. Examples include pyridyloxy, and pyrazinyloxy. “Substituted heteroaryloxy” refers to a heteroaryloxy, where the heteroaryl portion of the heteroaryloxy is substituted with the group or groups identified as substituents.

“Optional” or “optionally” means that the subsequently described event or circumstance may or may not occur, and that the description includes instances where said event or circumstance occurs and instances in which it does not. One of ordinary skill in the art would understand that, with respect to any molecule described as containing one or more optional substituents, that only sterically practical and/or synthetically feasible compounds are meant to be included. “Optionally substituted” refers to all subsequent modifiers in a term, for example in the term “optionally substituted arylC₁₋₈ alkyl,” optional substitution may occur on both the “C₁₋₈ alkyl” portion and the “aryl” portion of the molecule; and for example, optionally substituted alkyl includes optionally substituted cycloalkyl groups, which in turn are defined as including optionally substituted alkyl groups, potentially ad infinitum. A list of exemplary optional substitution are listed below in the definition of “substituted.”

“Saturated bridged ring system” refers to a bicyclic or polycyclic ring system that is not aromatic. Such a system may contain isolated or conjugated unsaturation, but not aromatic or heteroaromatic rings in its core structure (but may have aromatic substitution thereon). For example, hexahydro-furo[3,2-b]furan,2,3,3a,4,7,7a-hexahydro-1H-indene, 7-aza-bicyclo[2.2.1]heptane, and 1,2,3,4,4a,5,8,8a-octahydro-naphthalene are all included in the class “saturated bridged ring system.”

“Spirocyclyl” or “spirocyclic ring” refers to a ring originating from a particular annular carbon of another ring. For example, as depicted below, a ring atom of a saturated bridged ring system (rings B and B′), but not a bridgehead atom, can be a shared atom between the saturated bridged ring system and a spirocyclyl (ring A) attached thereto. A spirocyclyl can be carbocyclic or heteroalicyclic.

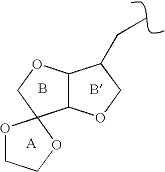

“Substituted” alkyl, aryl, and heterocyclyl, for example, refer respectively to alkyl, aryl, and heterocyclyl, wherein one or more (for example up to about five, in another example, up to about three) hydrogen atoms are each replaced by a substituent independently selected from: optionally substituted alkyl (for example, haloalkyl, alkoxylalkyl, hydroxyalkyl, nitroalkyl, and the like), optionally substituted aryl (for example, hydroxyphenyl, nitrophenyl, aminophenyl, halophenyl, alkylphenyl, and the like), optionally substituted arylalkyl (for example, 1-phenyl-ethyl), optionally substituted heterocyclylalkyl (for example, 1-pyridin-3-yl-ethyl), optionally substituted heterocyclyl (for example, 5-chloro-pyridin-3-yl and 1-methyl-piperidin-4-yl), optionally substituted alkoxy, alkylenedioxy (for example methylenedioxy), optionally substituted amino (for example, alkylamino and dialkylamino), optionally substituted amidino, optionally substituted aryloxy (for example, phenoxy), optionally substituted arylalkyloxy (for example, benzyloxy), carboxy (—CO₂H), carboalkoxy (that is, acyloxy or —OC(═O)R), carboxyalkyl (that is, esters or —CO₂R), carboxamido, benzyloxycarbonylamino (CBZ-amino), cyano, acyl, halogen, hydroxy, nitro, sulfanyl, sulfinyl, sulfonyl, thiol, halogen, hydroxy, oxo, carbamyl, acylamino, and sulfonamido.

“Sulfanyl” refers to the groups: —S-(optionally substituted alkyl), —S-(optionally substituted aryl), and —S-(optionally substituted heterocyclyl).

“Sulfinyl” refers to the groups: —S(O)—H, —S(O)-(optionally substituted alkyl), —S(O)-optionally substituted aryl), and —S(O)-(optionally substituted heterocyclyl).

“Sulfonyl” refers to the groups: —S(O₂)—H, —S(O₂)-(optionally substituted alkyl), —S(O₂)-optionally substituted aryl), —S(O₂)-(optionally substituted heterocyclyl), —S(O₂)-(optionally substituted alkoxy), —S(O₂)-optionally substituted aryloxy), and —S(O₂)-(optionally substituted heterocyclyloxy).

“Yield” for each of the reactions described herein is expressed as a percentage of the theoretical yield.

Some of the compounds of the invention may have imino, amino, oxo or hydroxy substituents off aromatic heterocyclyl systems. For purposes of this disclosure, it is understood that such imino, amino, oxo or hydroxy substituents may exist in their corresponding tautomeric form, i.e., amino, imino, hydroxy or oxo, respectively.

Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS).

The compounds of the invention, or their pharmaceutically acceptable salts, may have asymmetric carbon atoms, oxidized sulfur atoms or quaternized nitrogen atoms in their structure.

The compounds of the invention and their pharmaceutically acceptable salts may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

It is assumed that when considering generic descriptions of compounds of the invention for the purpose of constructing a compound, such construction results in the creation of a stable structure. That is, one of ordinary skill in the art would recognize that there can theoretically be some constructs which would not normally be considered as stable compounds (that is, sterically practical and/or synthetically feasible, supra).

When a particular group with its bonding structure is denoted as being bonded to two partners; that is, a divalent radical, for example, —OCH₂—, then it is understood that either of the two partners may be bound to the particular group at one end, and the other partner is necessarily bound to the other end of the particular group, unless stated explicitly otherwise. Stated another way, divalent radicals are not to be construed as limited to the depicted orientation, for example “—OCH₂—” is meant to mean not only “—OCH₂—” as drawn, but also “—CH₂O—.”

Methods for the preparation and/or separation and isolation of single stereoisomers from racemic mixtures or non-racemic mixtures of stereoisomers are well known in the art. For example, optically active (R)— and (S)-isomers may be prepared using chiral synthons or chiral reagents, or resolved using conventional techniques. Enantiomers (R— and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting on enantiomer to the other by asymmetric transformation. For a mixture of enantiomers, enriched in a particular enantiomer, the major component enantiomer may be further enriched (with concomitant loss in yield) by recrystallization.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

“Kinase-dependent diseases or conditions” refer to pathologic conditions that depend on the activity of one or more protein kinases. Kinases either directly or indirectly participate in the signal transduction pathways of a variety of cellular activities including proliferation, adhesion, migration, differentiation and invasion. Diseases associated with kinase activities include tumor growth, the pathologic neovascularization that supports solid tumor growth, and associated with other diseases where excessive local vascularization is involved such as ocular diseases (diabetic retinopathy, age-related macular degeneration, and the like) and inflammation (psoriasis, rheumatoid arthritis, and the like).

While not wishing to be bound to theory, phosphatases can also play a role in “kinase-dependent diseases or conditions” as cognates of kinases; that is, kinases phosphorylate and phosphatases dephosphorylate, for example protein substrates. Therefore compounds of the invention, while modulating kinase activity as described herein, may also modulate, either directly or indirectly, phosphatase activity. This additional modulation, if present, may be synergistic (or not) to activity of compounds of the invention toward a related or otherwise interdependent kinase or kinase family. In any case, as stated previously, the compounds of the invention are useful for treating diseases characterized in part by abnormal levels of cell proliferation (i.e. tumor growth), programmed cell death (apoptosis), cell migration and invasion and angiogenesis associated with tumor growth.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [neplrroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma [pinealoma], glioblastorna multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma), granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal lands: neuroblastoma. Thus, the term “cancerous cell” as provided herein, includes a cell afflicted by any one of the above-identified conditions.

“Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) wherein the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover compounds made either using standard organic synthetic techniques, including combinatorial chemistry or by biological methods, such as bacterial digestion, metabolism, enzymatic conversion, and the like.

“Treating” or “treatment” as used herein covers the treatment of a disease-state in a human, which disease-state is characterized by abnormal cellular proliferation, and invasion and includes at least one of: (i) preventing the disease-state from occurring in a human, in particular, when such human is predisposed to the disease-state but has not yet been diagnosed as having it; (ii) inhibiting the disease-state, i.e., arresting its development; and (iii) relieving the disease-state, i.e., causing regression of the disease-state. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

General Administration

Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages.

The compositions will include a conventional pharmaceutical carrier or excipient and a compound of the invention as the/an active agent, and, in addition, may include other medicinal agents, pharmaceutical agents, carriers, adjuvants, etc. Compositions of the invention may be used in combination with anticancer or other agents that are generally administered to a patient being treated for cancer. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

Utility of Compounds of the Invention as Screening Agents

To employ the compounds of the invention in a method of screening for candidate agents that bind to, for example KIAA1361 receptor kinase, the protein is bound to a support, and a compound of the invention is added to the assay. Alternatively, the compound of the invention is bound to the support and the protein is added. Classes of candidate agents among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate agent to, for example, KIAA1361 protein may be done in a number of ways. In one example, the candidate agent (the compound of the invention) is labeled, for example, with a fluorescent or radioactive moiety and binding determined directly. For example, thus may be done by attaching all or a portion of the KIAA1361 protein to a solid support, adding a labeled agent (for example a compound of the invention in which at least one atom has been replaced by a detectable isotope), washing off excess reagent, and determining whether the amount of the label is that present on the solid support. Various blocking and washing steps may be utilized as is known in the art.

By “labeled” herein is meant that the compound is either directly or indirectly labeled with a label which provides a detectable signal, for example, radioisotope, fluorescent tag, enzyme, antibodies, particles such as magnetic particles, chemiluminescent tag, or specific binding molecules, and the like. Specific binding molecules include pairs, such as biotin and streptavidin, digoxin and antidigoxin, and the like. For the specific binding members, the complementary member would normally be labeled with a molecule which provides for detection, in accordance with known procedures, as outlined above. The label can directly or indirectly provide a detectable signal.

In some embodiments, only one of the components is labeled. For example, KIAA1361 protein may be labeled at tyrosine positions using ¹²⁵I, or with fluorophores. Alternatively, more than one component may be labeled with different labels; using ¹²⁵I for the proteins, for example, and a fluorophor for the candidate agents.

The compounds of the invention may also be used as competitors to screen for additional drug candidates. “candidate bioactive agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. In the case where protein binding or activity is screened, some embodiments exclude molecules already known to bind to that particular protein. Exemplary embodiments of assays described herein include candidate agents, which do not bind the target protein in its endogenous native state, termed herein as “exogenous” agents. In one example, exogenous agents further exclude antibodies to KIAA1361.

Candidate agents can encompass numerous chemical classes, though typically they are organic molecules having a molecular weight of more than about 100 and less than about 2,500 daltons. Candidate agents comprise functional groups necessary for structural interaction with proteins, particularly hydrogen bonding and lipophilic binding, and typically include at least an amine, carbonyl, hydroxyl, ether, or carboxyl group, for example at least two of the functional chemical groups. The candidate agents often comprise cyclical carbon or heterocyclyl structures and/or aromatic or polyaromatic structures substituted with one or more of the above functional groups. Candidate agents are also found among biomolecules including peptides, saccharides, fatty acids, steroids, purines, pyrimidines, derivatives, structural analogs, or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In one example, the binding of the candidate agent is determined through the use of competitive binding assays. In this example, the competitor is a binding moiety known to bind to KIAA1361, such as an antibody, peptide, binding partner, ligand, etc. Under certain circumstances, there may be competitive binding as between the candidate agent and the binding moiety, with the binding moiety displacing the candidate agent.

In some embodiments, the candidate agent is labeled. Either the candidate agent, or the competitor, or both, is added first to KIAA1361 protein for a time sufficient to allow binding, if present. Incubations, may be performed at any temperature that facilitates optimal activity, typically between 4° C. and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In one example, the competitor is added first, followed by the candidate agent. Displacement of the competitor is an indication the candidate agent is binding to KIAA1361 and thus is capable of binding to, and potentially modulating, the activity of the KIAA1361. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate the candidate agent is bound to KIAA1361 with a higher affinity. Thus, if the candidate agent is labeled, the presence of the label on the support, coupled with a lack of competitor binding, may indicate the candidate agent is capable of binding to KIAA1361.

It may be of value to identify the binding site of KIAA1361. This can be done in a variety of ways. In one embodiment, once KIAA1361 has been identified as binding to the candidate agent, the KIAA1361 is fragmented or modified and the assays repeated to identify the necessary components for binding.

Modulation is tested by screening for candidate agents capable of modulating the activity of KIAA1361 comprising the steps of combining a candidate agent with KIAA1361, as above, and determining an alteration in the biological activity of the KIAA1361. Thus, in this embodiment, the candidate agent should both bind to (although this may not be necessary), and alter its biological or biochemical activity as defined herein. The methods include both in vitro screening methods and in vivo screening of cells for alterations in cell viability, morphology, and the like.

Alternatively, differential screening may be used to identify drug candidates that bind to native KIAA1361, but cannot bind to modified KIAA1361.

Positive controls and negative controls can be used in the assays. For example, all control and test samples are performed in at least triplicate to obtain statistically significant results. Incubation of samples is for a time sufficient for the binding of the agent to the protein. Following incubation, samples are washed free of non-specifically bound material and the amount of bound, generally labeled agent determined. For example, where a radiolabel is employed, the samples can be counted in a scintillation counter to determine the amount of bound compound.

A variety of other reagents can be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components can be added in any order that provides for the requisite binding.

One of ordinary skill in the art would understand that certain crystallized, protein-ligand complexes, in particular KIAA1361-, TAO-, or JIK-ligand complexes, and their corresponding x-ray structure coordinates can be used to reveal new structural information useful for understanding the biological activity of TAO kinase's as described herein. As well, the key structural features of the aforementioned proteins, particularly, the shape of the ligand binding site, are useful in methods for designing or identifying selective modulators of TAO kinase's and in solving the structures of other proteins with similar features. Ligands of such complexes may include compounds of the invention as described herein.

As well, one of ordinary skill in the art would appreciate that such suitable x-ray quality crystals can be used as part of a method of identifying a candidate agent capable of binding to and modulating the activity of TAO kinases. Such methods may be characterized by the following aspects: a) introducing into a suitable computer program, information defining a ligand binding domain of a TAO kinase in a conformation (e.g. as defined by x-ray structure coordinates obtained from suitable x-ray quality crystals as described above) wherein the computer program creates a model of the three dimensional structures of the ligand binding domain, b) introducing a model of the three dimensional structure of a candidate agent in the computer program, c) superimposing the model of the candidate agent on the model of the ligand binding domain, and d) assessing whether the candidate agent model fits spatially into the ligand binding domain. Aspects a-d are not necessarily carried out in the aforementioned order. Such methods may further entail: performing rational drug design with the model of the three-dimensional structure, and selecting a potential candidate agent in conjunction with computer modeling.

Additionally, one skilled in the art would appreciate that such methods may further entail: employing a candidate agent, so-determined to fit spatially into the ligand binding domain, in a biological activity assay for TAO kinase modulation, and determining whether said candidate agent modulates TAO kinase activity in the assay. Such methods may also include administering the candidate agent, determined to modulate TAO kinase activity, to a mammal suffering from a condition treatable by TAO kinase modulation, such as those described above.

Also, one skilled in the art would appreciate that compounds of the invention can be used in a method of evaluating the ability of a test agent to associate with a molecule or molecular complex comprising a ligand binding domain of a TAO kinase. Such a method may be characterized by the following aspects: a) creating a computer model of a TAO kinase binding pocket using structure coordinates obtained from suitable x-ray quality crystals of the TAO kinase, b) employing computational algorithms to perform a fitting operation between the test agent and the computer model of the binding pocket, and c) analyzing the results of the fitting operation to quantify the association between the test agent and the computer model of the binding pocket.

### ABBREVIATIONS AND THEIR DEFINITIONS

The following abbreviations and terms have the indicated meanings throughout:
Ac=acetyl
ATP=adenosine triphosphate
BNB=4-bromomethyl-3-nitrobenzoic acid
Boc=t-butyloxy carbonyl
br=broad
Bu=butyl
C=degrees Celsius
c-=cyclo
CBZ=carbobenzoxy=benzyloxycarbonyl
d=doublet
dd=doublet of doublet
dt=doublet of triplet
DBU=diazabicyclo[5.4.0]undec-7-ere
DCM=dichloromethane=methylene chloride=CH₂Cl₂
DCE=dichloroethylene
DEAD=diethyl azodicarboxylate
DIC=diisopropylcarbodiimide
DIEA=N,N-diisopropylethyl amine
DMA=N,N-dimethylacetamide
DMAP=4-N,N-dimethylaminopyridine
DMF=N,N-dimethylfonnamide
DMSO=dimethyl sulfoxide
DVB=1,4-divinylbenzene
EDC=1-(3-dimethylaminopropyl)-3-ethylcarbodiimide hydrochloride
EEDQ=2-ethoxy-1-ethoxycarbonyl-1,2-dihydroquinoline
EI=Electron Impact ionization
Et=ethyl
Fmoc=9-fluorenylmethoxycarbonyl
g=gram(s)
GC=gas chromatography
h or hr=hour(s)
HATU=0-(7-Azabenzotriazol-1-yl)-1,1,3,3-tetramethyluronium hexafluorophosphate
HMDS=hexamethyldisilazane
HOAc=acetic acid
HOBt=hydroxybenzotriazole
HPLC=high pressure liquid chromatography
L=liter(s)
M=molar or molarity
m=multiplet
Me=methyl
mesyl=methanesulfonyl
mg=milligram(s)
MHz=megahertz (frequency)
Min=minute(s)
mL=milliliter(s)
mM=millimolar
mmol=millimole(s)
mol=mole(s)
MS=mass spectral analysis
MTBE=methyl t-butyl ether
N=normal or normnality
NBS=N-bromosuccinimide
NCS=N-chlorosuccinimide
nM=nanomolar
NMO=N-methylmorpholine oxide
NMP=1-methyl-2-pyrrolidinone
NMR=nuclear magnetic resonance spectroscopy
PEG=polyethylene glycol
pEY=poly-glutamine, tyrosine
Ph=phenyl
PhOH=phenol
Pfp=pentafluorophenol
Pfpy=pentafluoropyridine
PPTS=pyridinium p-toluenesulfonate
Py=pyridine
PyBroP=bromo-tris-pyrrolidino-phosphonium hexafluorophosphate
q=quartet
RT=room temperature
Sat'd=saturated
s=singlet
s-=secondary
t-=tertiary
t or tr=triplet
TBDMS=t-butyldimethylsilyl
TES=triethylsilane
TFA=trifluoroacetic acid
THF=tetrahydrofuran
TMOF=trimethyl orthoformate
TMS=trimethylsilyl
tosyl=p-toluenesulfonyl
Trt=triphenylmethyl
uL=microliter(s)
uM=micromole(s) or micromolar
   Synthesis of Compounds

Schemes 1 and 2 depict general synthetic routes for compounds of the invention and is not intended to be limiting. Specific examples are described subsequently to this general synthetic description. With the descriptions of the general routes and the specific examples thereafter, one of ordinary skill in the art would be able to make compounds of the invention as described.

Scheme 1 shows that in general, compounds of formula I can be made, for example, via a linear route. For example, a commercially available amino acid or suitably protected amino acid derivative (i) (where X is ═O, A is for example —CH₂—, L¹ is either —OH or a suitable leaving group for amide bond formation, B is O, S, or N, and P¹ is a protecting group for B), is coupled with an amine represented by R¹(R²)NH to give intermediate amide (ii). Deprotection of B gives intermediate (iii). Intermediate (iii) is, for example, acylated to give compounds of formula I.

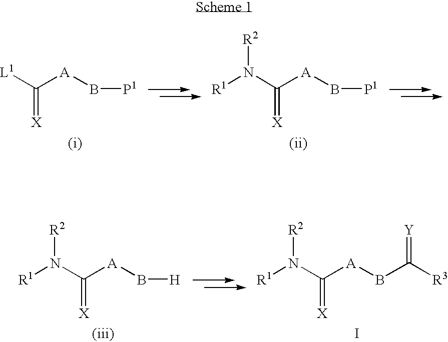

Scheme 2 shows that in general, compounds of formula VI can be made, for example, via a linear route. Form example, a commercially available amine or suitably protection amino acid derivative (i) (where R11, R12, X, Y and n are as defined above), is reacted with a carboxylic acid of (ii) (where Ar is as defined above) to arrive at a compound of of (iii).

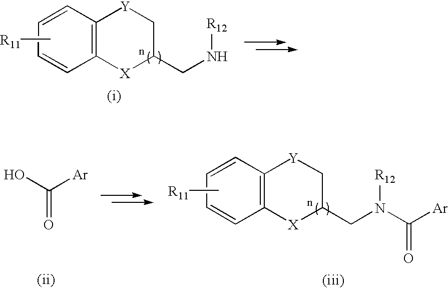

One of ordinary skill in the art would recognize that the descriptions associated with Schemes 1 and 2 are generalizations, and that there are other combinations of steps and approaches that can be used to make compounds of the invention. The examples that follow provide much more detailed descriptions of making exemplary compounds of the invention.

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety. Generally, but not necessarily, each example set out below describes a multi-step synthesis as outlined above.

### Example 1

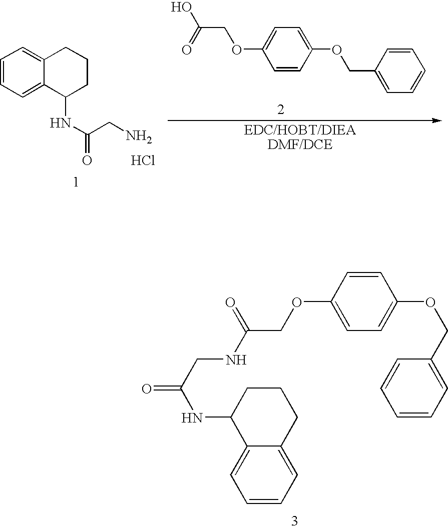

2-(4-Benzyloxy-phenoxy)-N-[(1,2,3,4-tetrahydro-naphthalen-1-ylcarbamoyl)-methyl]-acetamide 3. To a solution of 2-amino-N-(1,2,3,4-tetrahydro-naphthalen-1-yl)-acetamide hydrochloride 1 (67.9 mg, 0.283 mmol) in DMF (0.5 mL) and 1,2-Dichloroethane (2 mL) was added DIEA (150 μL, 0.859 mmol), HOBT (57.3 mg, 0.442 mmol), and (4-benzyloxyphenoxy)acetic acid 2 (74.0 mg, 0.287 mmol). With stirring, EDC (81.0 mg, 0.422 mmol) was added to the reaction at room temperature. The resulting slurry was stirred at room temperature for 20 hours before concentrating in vacuo and purifying via preparative HPLC. Obtained 3 as a white solid (58.4 mg, 46.7% yield). ¹H NMR (CDCl3); 7.43-7.37 (mult., 4H); 7.35-7.31 (mult., 2H); 7.24-7.22 (mult., 1H); 7.19-7.16 (mult., 2H); 7.11-7.09 (mult., 1H); 6.90-6.83 (mult., 4H); 6.19-6.17 (d, 1H); 5.21-5.16 (q, 1H); 5.01 (s, 2H); 4.44 (s, 2H), 4.04-4.02 (t, 2H); 2.84-2.71 (mult., 2H); 2.07-2.01 (mult., 1H); 1.84-1.74 (mult., 3H).

2-(4-Bromo-phenoxy)-N-[(1,2,3,4-tetrahydro-naphthalen-1-ylcarbamoyl)-methyl]-acetamide was prepared in a similar procedure as above from 2-amino-N-(1,2,3,4-tetrahydro-naphthalen-1-yl)-acetamide hydrochloride 1 and (4-bromophenoxy)acetic acid. 1H NMR (CDCl3) 7.39-7.36 (d, 2H); 7.31-7.25 (br., 1H); 7.22-7.17 (mult., 3H); 7.12-7.11 (d, 1H); 6.82-6.79 (d, 2H); 6.09-6.07 (d, 1H); 5.22-5.20 (q, 1H); 4.49 (s, 2H); 4.05-4.02 (t, 2H); 2.86-2.34 (mult., 2H); 2.05-2.02 (mult., 1H); 1.9-1.7 (br., 4H).

4′-Ethyl-biphenyl-4-carboxylic acid [(1,2,3,4-tetrahydro-naphthalen-1-ylcarbamoyl)-methyl]-amide was prepared in a similar procedure as above from 2-amino-N-(1,2,3,4-tetrahydro-naphthalen-1-yl)-acetamide hydrochloride 1 and 4′-ethyl-biphenyl-4-carboxylic acid. 1H NMR (CDCl3); 7.82-7.99 (d, 2H); 7.63-7.61 (d, 2H); 7.53-7.52 (d, 2H); 7.29-7.27 (d, 2H); 7.17-7.14 (mult., 2H); 7.09-7.03 (mult., 2H); 6.41-6.39 (d, 1H); 5.22-5.19 (mult., 1H); 4.19-4.18 (d, 2H); 2.84-2.75 (mult, 2H); 2.73-2.68 (q, 2H); 2.10-2.04 (mult., 1H); 1.87-1.84 (mult., 3H); 1.30-1.24 (t, 3H).

### Example 2

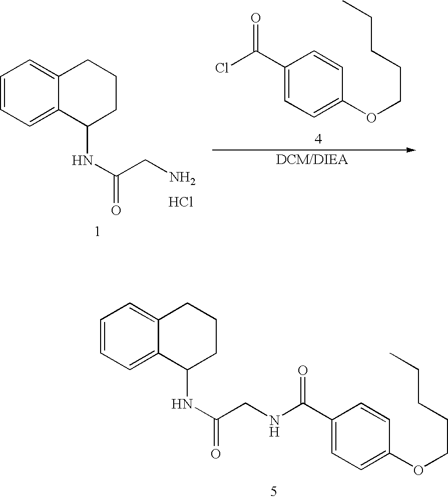

4-Pentyloxy-N-[(1,2,3,4-tetrahydro-naphthalen-1-ylcarbamoyl)-methyl]-benzamide 5. To a stirred solution of 1 (84.8 mg, 0.352 mmol) in DCM (2 mL) and DIEA (180 μL, 1.03 mmol) was added 4-pentyloxybenzoyl chloride 4 (82.0 mg, 0.362 mmol). The resulting slurry was stirred at room temperature for 14 hours before diluting with DCM (10 mL). The diluted mixture was washed with HCl (aq., 0.5M), NaHCO₃ (aq., satd.) and brine. After drying over MgSO₄ the solvent was removed in vacuo, yielding yellow solids which were further purified by flash chromatography (1:1 Hexanes:EtOAc). Product 5 was obtained as a white solid (63.5 mg, 45.7% yield). 1H NMR (CDCl3) 7.67-7.63 (dt, 2H); 7.24-7.22 (dd, 1H); 7.17-7.10 (dq, 2H); 7.08-7.06 (d, 1H); 7.02-7.00 (t, 1H); 6.88-6.84 (dt, 2H); 6.77-6.75 (d, 1H); 5.20-5.16 (q, 1H); 4.16-4.15 (d, 2H); 4.00-3.96 (t, 2H); 2.81-2.71 (mult., 2H); 2.07-2.01 (mult., 1H); 1.82-1.77 (mult., 5H), 1.48-1.36 (mult., 4H); 0.96-0.92 (t, 3H).

### Example 3

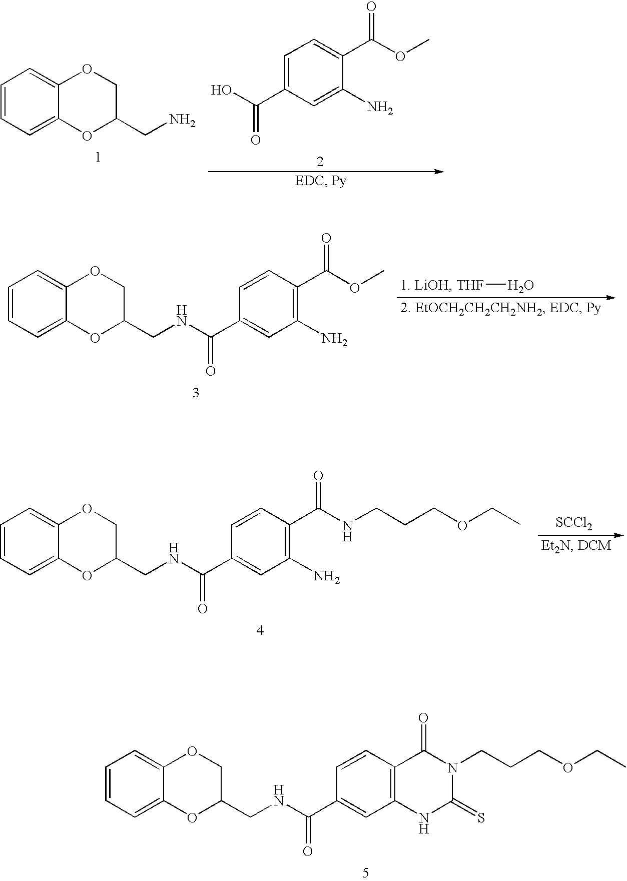

2-Amino-N-(2,3-dihydro-benzo[1,4]dioxin-2-ylmethyl)-terephthalamic acid methyl ester 3. To a solution of 1 (1.65 g, 10 mmol), and 2 (1.95 g, 10 mmol) in pyridine (30 mL) was added EDC (2.88 g, 15 mmol) and the mixture was stirred at room temperature for 16 h. Pyridine was removed in vacuo and the residue was dissolved in EtOAc, washed sequentially with 3M HCl, satd. NaHCO₃, satd. NaCl, and then dried over Na₂SO₄. The solvent was removed in vacuo to give compound 3 (3.08 g, 90%) pure as a yellow solid: MS: m/z 343.20 (M+H)⁺; ¹H NMR (CDCl₃) δ 7.91 (d, 1H), 7.11 (d, 1H), 6.93-6.84 (m, 1H), 6.58 (t, 1H), 5.87 (bs, 2H), 4.42-4.36 (m, 1H), 4.34 (dd, 1H), 4.01 (dd, 1H), 3.89 (s, 3H), 3.88-3.83 (m, 1H), 3.74-3.65 (m, 1H).

2-Amino-N4-(2.3-dihydro-benzo[1,4]dioxin-2-ylmethyl)-N1-(3-ethoxy-propyl)-terephthalamide 4. To a solution of 3 (3.08 g, 9 mmol) in THF (75 ml) at room temperature was added a solution of LiOH (647 mg in 25 ml H₂O). The mixture was stirred at room temperature for 16 h. The reaction was treated with additional LiOH (1 g) and was stirred for another 24 h. The mixture was concentrated in vacuo, and the residue dissolved in H₂O and extracted once with Et₂O. The aqueous solution was then acidified with 3M HCl to pH ˜4, extracted with EtOAc, and dried over Na₂SO₄. The solvent was then removed in vacuo to give the carboxylic acid as a pink solid (2.75 g, 93%). The crude acid was dissolved in pyridine (60 mL) and was treated with 3-ethoxypropylamine (864 mg, 8.38 mmol), and EDC (2.41 g, 12.55 mmol) and was stirred at room temperature for 16 h. Pyridine was removed in vacuo and the residue worked up as described above to give compound 4 (935 mg, 27%) pure as a white solid: MS: m/z 414.20 (M+H)⁺; ¹H NMR (CDCl₃) δ 7.47 (d, 1H), 7.16 (d, 1H), 7.00 (dd, 1H), 6.87-6.77 (m, 4H), 4.37-4.28 (m, 2H), 3.96 (dd, 1H), 3.70-3.60 (m, 2H), 3.53 (t, 2H), 3.50 (q, 2H), 3.42 (t, 2H), 1.90-1.82 (m, 2H), 1.20 (t, 3H).

3-(3-Ethoxy-propyl)-4-oxo-2-thioxo-1,2,3,4-tetrahydro-quinazoline-7-carboxylic acid (2,3-dihydro-benzo[1,4]dioxin-2-ylmethyl)-amide 5. To a solution of 4 (130 mg, 0.314 mmol) in DCM (20 mL) at 0° C. was added dropwise a solution of thiophosgene (1M in DCM, 0.315 mL, 0.315 mmol). The mixture was then allowed to warm up to room temperature and stirred at room temperature for 1 h. The mixture was concentrated and the residue purified by preparative HPLC to give compound 5 (25.2 mg, 18%) as a white solid: MS: m/z 456.18 (M+H)⁺; ¹H NMR (CDCl₃) δ 11.36 (s, 1H), 8.35 (s, 1H), 8.17 (d, 2H), 7.48 (dd, 1H), 6.94-6.82 (m, 5H), 4.66-4.61 (m, 2H), 4.51-4.46 (m, 2H), 4.10-4.03 (m, 1H), 3.99-3.92 (m, 1H), 3.58 (t, 2H), 3.48 (q, 2H), 2.15-2.06 (m, 2H), 1.15 (t, 3H).

### Example 4

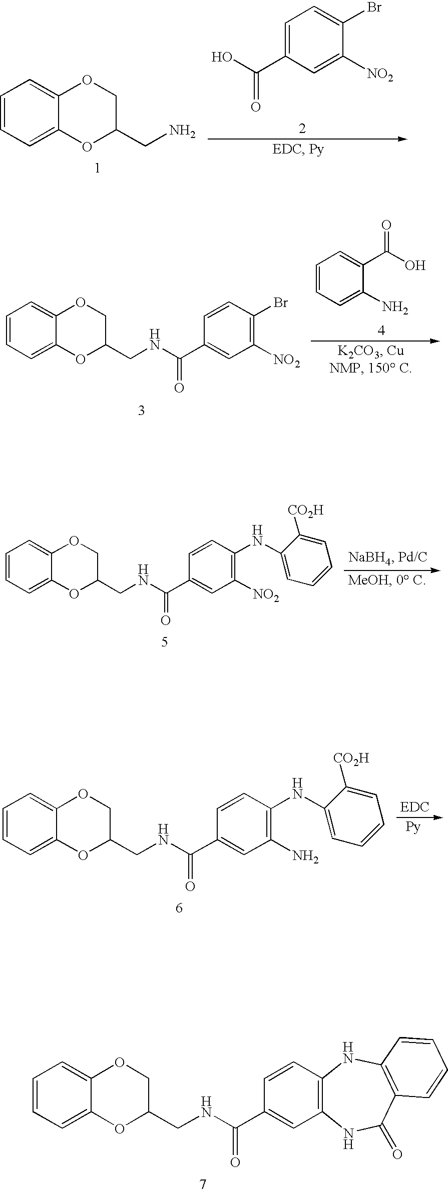

4-Bromo-N-(2,3-dihydro-benzo[1,4]dioxin-2-ylmethyl)-3-nitro-benzamide 3. To a solution of 1 (1.65 g, 10 mmol) and 2 (2.46 g, 10 mmol) in pyridine (80 mL) was added and EDC (2.87 g, 15 mmol), and mixture was stirred at room temperature for 16 h. Pyridine was removed in vacuo and the residue was dissolved in EtOAc, washed sequentially with 3M HCl, satd. NaHCO₃, and satd. NaCl, and then dried over Na₂SO₄. The solvent was removed in vacuo to give compound 3 (3.19 g, 81%) as a yellow solid: MS: m/z 393.03 (M+H)⁺.

2-{4-[(2,3-Dihydro-benzo[1,4]dioxin-2-ylmethyl)-carbamoyl]-2-nitro-phenylamino}-benzoic acid 5. A solution of 3 (3.19 g, 8.12 mmol), and 4 (1.05 g, 7.66 mmol) in NMP (6 mL) was treated with K₂CO₃ (1.57 g, 10.40 mmol) and the mixture was stirred in an open pressure tube until gas evolution ceased. Copper powder (3 micron, 40 mg) was added and the mixture was then heated in a sealed tube at 150° C. for about 30 min. The mixture was cooled down to room temperature, diluted with H₂O, filtered, and the filtrate was acidified with 3M HCl to precipitate a solid. The solid was filtered, washed with H₂O, and dried. The crude product was purified by flash chromatography (1:1 Hexanes:EtOAc) to give compound 5 (1.31 g, 38%) as a yellow solid: MS: m/z 450.14 (M+H)⁺; ¹H NMR (CD₃OD) δ 8.65 (d, 1H), 8.00 (dd, 1H), 7.88 (dd, 1H), 7.57 (d, 1H), 7.54-7.43 (m, 2H), 7.11-7.06 (m, 1H), 6.79-6.68 (m, 4H), 4.30-4.20 (m, 2H), 3.89 (dd, 1H), 3.61-3.56 (m, 2H).

2-{2-Amino-4-[(2,3-dihydro-benzo[1,4]dioxin-2-ylmethyl)-carbamoyl]-phenylamino}-benzoic acid 6. To a suspension of Pd/C (30 mg) in MeOH (100 mL) under N₂ was added 5 (210 mg, 0.47 mmol). The mixture was cooled to 0° C. and NaBH₄ (250 mg) was added in several portions. The mixture was stirred at 0° C. for 1 h, filtered, and concentrated in vacuo. The residue was diluted with H₂O, acidified with 3M HCl to pH ˜4, and then extracted with EtOAc, dried over Na₂SO₄, and concentrated to give compound 6 (192 mg, 98%) as a pink solid: MS: m/z 420.22 (M+H)⁺.

11-Oxo-10,11-dihydro-5H-dibenzo[b,e][1,4]diazepine-8-carboxylic acid (2,3-dihydro-benzo[1,4]dioxin-2-ylmethyl)-amide 7. To a solution of 6 (192 mg, 0.46 mmol) in pyridine (30 mL) was added EDC (180 mg, 0.94 mmol), and the mixture was stirred at room temperature for 16 h. Pyridine was removed in vacuo and the residue was dissolved in EtOAc, washed sequentially with 3M HCl, satd. NaHCO₃, and satd. NaCl, and then dried over Na₂SO₄. The solvent was removed in vacuo, and the crude product was purified by flash chromatography (1:1 Hexanes:EtOAc) to afford compound 7 (164.3 mg, 89%) as a yellow solid: MS: m/z 402.17 (M+H)⁺; ¹H NMR (CD₃OD) δ 7.76 (dd, 1H), 7.48 (dd, 1H), 7.42 (d, 1H), 7.38-7.32 (m, 1H), 7.01-6.76 (m, 7H), 4.36-4.29 (m, 2H), 3.96 (dd, 1H), 3.70-3.59 (m, 2H).

### Example 5

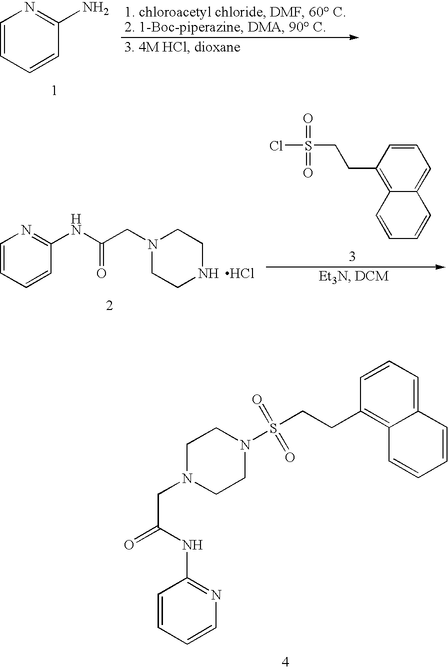

2-Piperazin-1-yl-N-pyridin-2-yl-acetamide hydrochloride 2. To a solution of 1 (941 mg, 10 mmol) in dry DMF (5 mL) at room temperature was added dropwise chloroacetyl chloride (1.69 g, 15 mmol). A solid mixture quickly formed after addition of the acid chloride, which was then heated at 60° C. for 30 min. The mixture was cooled down to room temperature, diluted with Et₂O, and the solid was filtered. The solid was treated with satd. NaHCO₃, washed with H₂O, and air-dried to give 2-chloro-N-pyridin-2-yl-acetamide as a solid (1.05 g, 62%). MS: m/z 171.16 (M+H)⁺. To a solution of the crude 2-chloro-N-pyridin-2-yl-acetamide (171 mg, 1 mmol) in DMA (2 mL) was added 1-Boc-piperazine (373 mg, 2 mmol) and the solution was heated at 90° C. for 2 h. The mixture was cooled to room temperature, diluted with H₂O, treated with satd. NaHCO₃ and extracted with EtOAc. The organic extract was dried over MgSO₄, and concentrated in vacuo to give 4-(Pyridin-2-ylcarbamoylmethyl)-piperazine-1-carboxylic acid tert-butyl ester (154 mg, 48%), which was treated with 4M HCl in dioxane (5 mL) at room temperature for 16 h. The solvent was removed and the residue vacuum-dried to give 2 as a light pink solid. MS: m/z 221.26 (M+H)⁺.

2-[4-(2-Naphthalen-1-yl-ethanesulfonyl)-piperazin-1-yl]-N-pyridin-2-yl-acetamide 4. To a solution of 2 in CH₂Cl₂ (10 mL), at room temperature was added Et₃N (0.33 mL, 2.34 mmol) followed by 3 (120 mg, 0.47 mmol) and the mixture was stirred at room temperature for 1 h and concentrated in vacuo. The crude product was purified by preparative HPLC to give the compound 4 (20 mg, 10%) as a white solid. MS: m/z 439.23 (M+H)⁺; ¹H NMR (DMSO-d₆): δ 9.96 (s, 1H), 8.31-8.27 (m, 1H), 8.08 (d, 1H), 8.03 (d, 1H), 7.97 (d, 1H), 7.85 (d, 1H), 7.83-7.76 (m, 1H), 7.65-7.44 (m, 4H), 7.14-7.09 (m, 1H), 3.51-3.38 (m, 4H), 3.32-3.26 (m, 4H), 3.26 (s, 2H), 2.66-2.58 (m, 4H).

Assays

Kinase assays were performed by measurement of incorporation of γ-³³P ATP into immobilized myelin basic protein (MBP). High binding white 384 well plates (Greiner) were coated with MBP (Sigma #M-1891) by incubation of 60 ul/well of 20 μg/ml MBP in Tris-buffered saline (TBS; 50 mM Tris pH 8.0, 138 mM NaCl, 2.7 mM KCl) for 24 hours at 4° C. Plates were washed 3× with 100 μl TBS. Kinase reactions were carried out in a total volume of 34 μl in kinase buffer (5 mM Hepes pH 7.6, 15 mM NaCl, 0.01% bovine gamma globulin (Sigma #I-5506), 10 mM MgCl₂, 1 mM DTT, 0.02% TritonX-100). Compound dilutions were performed in DMSO and added to assay wells to a final DMSO concentration of 1%. Each data point was measured in duplicate, and at least two duplicate assays were performed for each individual compound determination. Enzyme was added to final concentrations of 10 nM or 20 nM, for example. A mixture of unlabeled ATP and γ-³³P ATP was added to start the reaction (2×10⁶ cpm of γ-³³P ATP per well (3000 Ci/mmole) and either 10 μM or 30 μM unlabeled ATP, typically. The reactions were carried out for 1 hour at room temperature with shaking. Plates were washed 7× with TBS, followed by the addition of 50 μl/well scintillation fluid (Wallac). Plates were read using a Wallac Trilux counter. This is only one format of such assays, various other formats are possible, as known to one of ordinary skill in the art.

The above assay procedure can be used to determine the IC₅₀ for inhibition and/or the inhibition constant, Kᵢ. The IC₅₀ is defined as the concentration of compound required to reduce the enzyme activity by 50% under the conditions of the assay. Exemplary compositions have IC₅₀'s of, for example, less than about 100 μM, less than about 10 μM, less than about 1 μM, and further for example having IC₅₀'s of less than about 100 nM, and still further, for example, less than about 10 nM. The Kᵢ for a compound may be determined

$$V = {V}_{max} {E}_{0} [ 1 - \frac{\begin{matrix}( {E}_{0} + {I}_{0} + {K}_{d} ) - \\ \sqrt{{( {E}_{0} + {I}_{0} + {K}_{d} )}^{2} - 4 {E}_{0}} {I}_{0}\end{matrix}}{2 {E}_{0}} ] \qquad Equation ( 1 )$$

from the IC₅₀ based on three assumptions. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to equation (1) below; where V is the observed rate, Vₘₐₓ, is the rate of the free enzyme, I₀ is the inhibitor concentration, E₀ is the enzyme concentration, and K_{d} is the dissociation constant of the enzyme-inhibitor complex.

Kinase Specific Assays:

Kinase activity and compound inhibition are investigated using one or more of the three assay formats described below. The ATP concentrations for each assay are selected to be close to the Michaelis-Menten constant (K_{M}) for each individual kinase. Dose-response experiments are performed at 10 different inhibitor concentrations in a 384-well plate format. The data are fitted to four-parameter equation (2) below; where Y is the observed signal, X is the inhibitor concentration, Min is the background signal in the absence of enzyme (0% enzyme activity), Max is the signal in the absence of inhibitor (100% enzyme activity), IC₅₀ is the inhibitor concentration at 50% enzyme inhibition and H represents the empirical Hill's slope to measure the cooperativity. Typically H is close to unity.
Y=Min+(Max−Min)/(1+(X/IC₅₀)ˆH) Equation (2)
KIAA 1361 Assay:

KIAA 1361 biochemical activity was assessed using a Luciferase-Coupled Chemiluminescent Kinase assay (LCCA) format. Kinase activity was measured as the percent ATP remaining following the kinase reaction. Remaining ATP was detected by luciferase-luciferin-coupled chemiluminescence. Specifically, the reaction was initiated by mixing test compounds, 2 μM ATP, 10 μM MBP and 12 nM KIAA 1361 (baculovirus expressed human KIAA 1361 kinase domain 2-322) in a 20 uL assay buffer (20 mM Tris-HCL pH7.5, 10 mM MgCl₂, 0.02% Triton X-100, 100 mM DTT, 2 mM MnCl₂). The mixture is incubated at ambient temperature for 2 hours after which 20 uL luciferase-luciferin mix is added and the chemiluminescent signal read using a Wallac Victor² reader. The luciferase-luciferin mix consists of 50 mM HEPES, pH 7.8, 8.5 ug/mL oxalic acid (pH 7.8), 5 (or 50) mM DTT, 0.4% Triton X-100, 0.25 mg/mL coenzyme A, 63 uM AMP, 28 ug/mL luciferin and 40,000 units of light/mL luciferase.

JIK Assay

JIK biochemical activity was assessed using a Luciferase-Coupled Chemiluminescent Kinase assay (LCCA) format. Kinase activity was measured as the percent ATP remaining following the kinase reaction. Remaining ATP was detected by luciferase-luciferin-coupled chemiluminescence. Specifically, the reaction was initiated by mixing test compounds, 2 μM ATP, 10 μM MBP and 6 nM JIK (baculovirus expressed human KIAA 1361 kinase domain2-318) in a 20 uL assay buffer (20 mM Tris-HCL pH7.5, 10 mM MgCl₂, 0.02% Triton X-100, 100 mM DTT, 2 mM MnCl₂). The mixture is incubated at ambient temperature for 2 hours after which 20 uL luciferase-luciferin mix is added and the chemiluminescent signal read using a Wallac Victor² reader. The luciferase-luciferin mix consists of 50 mM HEPES, pH 7.8, 8.5 ug/mL oxalic acid (pH 7.8), 5 (or 50) mM DTT, 0.4% Triton X-100, 0.25 mg/mL coenzyme A, 63 uM AMP, 28 ug/mL luciferin and 40,000 units of light/mL luciferase.

TAO Assay

TAO biochemical activity was assessed using a Luciferase-Coupled Chemiluminescent Kinase assay (LCCA) format. Kinase activity was measured as the percent ATP remaining following the kinase reaction. Remaining ATP was detected by luciferase-luciferin-coupled chemiluminescence. Specifically, the reaction was initiated by mixing test compounds, 2 μM ATP, 10 μM MBP and 12 nM TAO (baculovirus expressed human KIAA 1361 kinase domain 2-322) in a 20 uL assay buffer (20 mM Tris-HCL pH7.5, 10 mM MgCl₂, 0.02% Triton X-100, 100 mM DTT, 2 mM MnCl₂). The mixture is incubated at ambient temperature for 2 hours after which 20 uL luciferase-luciferin mix is added and the chemiluminescent signal read using a Wallac Victor² reader. The luciferase-luciferin mix consists of 50 mM BEPES, pH 7.8, 8.5 ug/mL oxalic acid (pH 7.8), 5 (or 50) mM DTT, 0.4% Triton X-100, 0.25 mg/nL coenzyme A, 63 uM AMP, 28 ug/mL luciferin and 40,000 units of light/mL luciferase.

Structure Activity Relationships

Table 2 shows structure activity relationship data for selected compounds of the invention. Inhibition is indicated as IC₅₀ with the following key: A=IC₅₀ less than 50 nM, B=IC₅₀ greater than 50 nM, but less than 500 nM, C=IC₅₀ greater than 500 nM, but less than 5000 nM, and D=IC₅₀ equal to, or greater than 5,000 nM.

TABLE 2

| Entry | Name | KIAA1361 | JIK | TAO |
|---|---|---|---|---|
| 1 | N-[2-oxo-2-(1,2,3,4- | A | A | B |
| | tetrahydronaphthalen-1- | | | |
| | ylamino)ethyl]-4- | | | |
| | (pentyloxy)benzamide | | | |
| 2 | N˜2˜-[({4- | B | A | B |
| | [(phenylmethyl)oxy]phenyl}oxy)acet | | | |
| | yl]-N-(1,2,3,4-tetrahydronaphthalen- | | | |
| | 1-yl)glycinamide | | | |
| 3 | N˜2˜-{[(4-bromophenyl)oxy]acetyl}- | B | A | B |
| | N-(1,2,3,4-tetrahydronaphthalen-1- | | | |
| | yl)glycinamide | | | |
| 4 | 4′-ethyl-N-[2-oxo-2-(1,2,3,4- | A | A | A |
| | tetrahydronaphthalen-1- | | | |
| | ylamino)ethyl]biphenyl-4- | | | |
| | carboxamide | | | |
| 5 | 4′-ethyl-N-[1-methyl-2-oxo-2- | A | A | B |
| | (1,2,3,4-tetrahydronaphthalen-2- | | | |
| | ylamino)ethyl]biphenyl-4- | | | |
| | carboxamide | | | |
| 6 | 4-(hexyloxy)-N-[2-oxo-2-(1,2,3,4- | A | A | B |
| | tetrahydronaphthalen-1- | | | |
| | ylamino)ethyl]benzamide | | | |
| 7 | 2-cyclopentyl-N-[2-oxo-1-pyridin-3- | B | A | B |
| | yl-2-(1,2,3,4-tetrahydronaphthalen- | | | |
| | 1-ylamino)ethyl]-2-phenylacetamide | | | |
| 8 | 4-(heptyloxy)-N-[2-oxo-2-(1,2,3,4- | B | B | B |
| | tetrahydronaphthalen-1- | | | |
| | ylamino)ethyl]benzamide | | | |
| 9 | N-[1-methyl-2-oxo-2-(1,2,3,4- | B | B | C |
| | tetrahydronaphthalen-2- | | | |
| | ylamino)ethyl]-4- | | | |
| | (pentyloxy)benzamide | | | |
| 10 | 4-hexyl-N-[2-oxo-2-(1,2,3,4- | B | B | B |
| | tetrahydronaphthalen-1- | | | |
| | ylamino)ethyl]benzamide | | | |
| 11 | N-[2-oxo-2-(1,2,3,4- | B | B | B |
| | tetrahydronaphthalen-1- | | | |
| | ylamino)ethyl]-4-pentylbenzamide | | | |
| 12 | 4-heptyl-N-[2-oxo-2-(1,2,3,4- | B | B | C |
| | tetrahydronaphthalen-1- | | | |
| | ylamino)ethyl]benzamide | | | |
| 13 | Nalpha-{[5,6-bis(methyloxy)-1H- | B | A | C |
| | indol-2-yl]carbonyl}-N-(1,2,3,4- | | | |
| | tetrahydronaphthalen-1- | | | |
| | yl)tryptophanamide | | | |
| 14 | Nalpha-{[4- | B | B | C |
| | (butyloxy)phenyl]carbonyl}-N- | | | |
| | (1,2,3,4-tetrahydronaphthalen-1- | | | |
| | yl)tryptophanamide | | | |
| 15 | 5-{(2E)-3-[3,4- | B | A | C |
| | bis(methyloxy)phenyl]prop-2-enoyl}- | | | |
| | N-[2-(3-chlorophenyl)ethyl]-4,5,6,7- | | | |
| | tetrahydro-3H-imidazo[4,5- | | | |
| | c]pyridine-6-carboxamide | | | |
| 16 | N-[2-oxo-1-pyridin-3-yl-2-(1,2,3,4- | B | B | B |
| | tetrahydronaphthalen-1- | | | |
| | ylamino)ethyl]-4-(1H-1,2,4-triazol-1- | | | |
| | yl)benzamide | | | |
| 17 | Nalpha-{(2E)-3-[3,4- | B | B | C |
| | bis(methyloxy)phenyl]prop-2-enoyl}- | | | |
| | N-(1,2,3,4-tetrahydronaphthalen-1- | | | |
| | yl)tryptophanamide | | | |
| 18 | 4-(butyloxy)-N-[1-methyl-2-oxo-2- | B | B | C |
| | (1,2,3,4-tetrahydronaphthalen-2- | | | |
| | ylamino)ethyl]benzamide | | | |
| 19 | N-[1-methyl-2-oxo-2-(1,2,3,4- | B | B | C |
| | tetrahydronaphthalen-2- | | | |
| | ylamino)ethyl]-4- | | | |
| | [(phenylmethyl)oxy]benzamide | | | |
| 20 | 3,5-dimethyl-N-[2-oxo-2-(1,2,3,4- | B | B | C |
| | tetrahydronaphthalen-1- | | | |
| | ylamino)ethyl]-4- | | | |
| | [(phenylmethyl)oxy]benzamide | | | |
| 21 | N˜2˜-({[4- | B | B | D |
| | (butyloxy)phenyl]amino}carbonyl)- | | | |
| | N-(2-phenylethyl)-O- | | | |
| | (phenylmethyl)serinamide | | | |
| 22 | (2S)-N˜1˜-(4-butylphenyl)-N˜2˜- | B | B | C |
| | [(3,4- | | | |
| | dichlorophenyl)methyl]pyrrolidine- | | | |
| | 1,2-dicarboxamide | | | |
| 23 | N-[6-(methyloxy)-1,3-benzothiazol- | B | B | C |
| | 2-yl]-4-{[(4-oxo-5,6,7,8- | | | |
| | tetrahydro[1]benzothieno[2,3- | | | |
| | d]pyrimidin-3(4H)- | | | |
| | yl)acetyl]amino}butanamide | | | |
| 24 | (2S)-N˜2˜-[(3,4- | B | B | C |
| | dichlorophenyl)methyl]-N˜1˜-[4-(1- | | | |
| | methylethyl)phenyl]pyrrolidine-1,2- | | | |
| | dicarboxamide | | | |
| 25 | N˜2˜-{[(4-bromophenyl)oxy]acetyl}- | B | B | C |
| | N-[(3,4- | | | |
| | dichlorophenyl)methyl]glycinamide | | | |
| 26 | N-[2-oxo-1-pyridin-3-yl-2-(1,2,3,4- | C | B | C |
| | tetrahydronaphthalen-1- | | | |
| | ylamino)ethyl]-4-[(E)- | | | |
| | phenyldiazenyl]benzamide | | | |
| 27 | 4-(butyloxy)-N-[2-oxo-2-(1,2,3,4- | C | B | C |
| | tetrahydronaphthalen-1- | | | |
| | ylamino)ethyl]benzamide | | | |
| 28 | N˜2˜-({[3- | C | B | C |
| | (methyloxy)phenyl]oxy}acetyl)-N- | | | |
| | (1,2,3,4-tetrahydronaphthalen-1- | | | |
| | yl)glycinamide | | | |
| 29 | 4-butyl-N-[2-oxo-2-(1,2,3,4- | C | C | D |
| | tetrahydronaphthalen-1- | | | |
| | ylamino)ethyl]benzamide | | | |
| 30 | N˜2˜-{(2E)-3-[3,4- | C | B | D |
| | bis(methyloxy)phenyl]prop-2-enoyl]- | | | |
| | N-(diphenylmethyl)-O- | | | |
| | (phenylmethyl)serinamide | | | |
| 31 | N-(1,2,3,4-tetrahydronaphthalen-1- | C | C | C |
| | yl)-N˜2˜-[({4- | | | |
| | [(trifluoromethyl)oxy]phenyl}oxy) | | | |
| | acetyl]glycinamide | | | |
| 32 | N-{3-methyl-1-[(1,2,3,4- | C | C | D |
| | tetrahydronaphthalen-1- | | | |
| | ylamino)carbonyl]butyl}biphenyl-4- | | | |
| | carboxamide | | | |
| 33 | N˜2˜-({[4-(1,1- | C | C | D |
| | dimethylethyl)phenyl]oxy}acetyl)-N- | | | |
| | (1,2,3,4-tetrahydronaphthalen-1- | | | |
| | yl)glycinamide | | | |
| 34 | N˜2˜-{[(4-chlorophenyl)oxy]acetyl}- | D | C | D |
| | N-(1,2,3,4-tetrahydronaphthalen-1- | | | |
| | yl)glycinamide | | | |
| 35 | N˜2˜-{[4- | D | B | C |
| | (pentyloxy)phenyl]carbonyl}-N | | | |
| | (1,2,3,4-tetrahydronaphthalen-1- | | | |
| | yl)leucinamide | | | |
| 36 | 4-(hexyloxy)-N-(1-methyl-2-oxo-2- | D | B | D |
| | (1,2,3,4-tetrahydronaphthalen-2- | | | |
| | ylamino)ethyl]benzamide | | | |
| 37 | 2-{[6-(methyloxy)-1,3-benzothiazol- | A | A | A |
| | 2-yl]amino}-2-oxoethyl 3-phenyl-3- | | | |
| | [(phenylcarbonyl)amino]propanoate | | | |
| 38 | N-[(3,4-dichlorophenyl)methyl]-2- | A | A | B |
| | {[4-(4-pyridin-2-ylpyrimidin-2- | | | |
| | yl)phenyl]oxy}acetamide | | | |
| 39 | N-(2,3-dihydro-1,4-benzodioxin-2- | B | B | B |
| | ylmethyl)-3-[3-(ethyloxy)propyl]-4- | | | |
| | oxo-2-thioxo-1,2,3,4- | | | |
| | tetrahydroquinazoline-7- | | | |
| | carboxamide | | | |
| 40 | N˜2˜-[(5,6-dimethyl-4- | B | B | C |
| | oxothieno[2,3-d]pyrimidin-3(4H)- | | | |
| | yl)acetyl]-N-[6-(methyloxy)-1,3- | | | |
| | benzothiazol-2-yl]glycinamide | | | |
| 41 | 2-{4-[(2-naphthalen-1- | A | A | B |
| | ylethyl)sulfonyl]piperazin-1-yl}-N- | | | |
| | pyridin-2-ylacetamide | | | |
| 42 | N-(2,3-dihydro-1,4-benzodioxin-2- | B | A | B |
| | ylmethyl)-11-oxo-10,11-dihydro-5H- | | | |
| | dibenzo[b,e][1,4]diazepine-8- | | | |
| | carboxamide | | | |
| 43 | N-[2-oxo-2-(1,2,3,4- | A | A | A |
| | tetrahydronaphthalen-1- | | | |
| | ylamino)ethyl]biphenyl-4- | | | |
| | carboxamide | | | |
| 44 | N-[2-oxo-2-(1,2,3,4- | C | C | D |
| | tetrahydronaphthalen-1- | | | |
| | ylamino)ethyl]naphthalene-2- | | | |
| | carboxamide | | | |
| 45 | N-[2-oxo-2-(1,2,3,4- | C | B | C |
| | tetrahydronaphthalen-1- | | | |
| | ylamino)ethyl]-4- | | | |
| | [(trifluoromethyl)oxy]benzamide | | | |
| 46 | N-[2-oxo-2-(1,2,3,4- | D | D | D |
| | tetrahydronaphthalen-1- | | | |
| | ylamino)ethyl]quinoline-3- | | | |
| | carboxamide | | | |
| 47 | N˜2˜-({[3,5- | D | D | D |
| | bis(trifluoromethyl)phen- | | | |
| | yl]amino}carbonyl)-N-(1,2,3,4- | | | |
| | tetrahydronaphthalen-1- | | | |
| | yl)glycinamide | | | |
| 48 | N˜2˜-{[(3- | D | D | D |
| | ethylphenyl)amino]carbonyl}-N- | | | |
| | (1,2,3,4-tetrahydronaphthalen-1- | | | |
| | yl)glycinamide | | | |
| 49 | N-[2-oxo-2-(1,2,3,4- | B | B | B |
| | tetrahydronaphthalen-1- | | | |
| | ylamino)ethyl]-4- | | | |
| | (phenyloxy)benzamide | | | |
| 50 | 4-cyclohexyl-N-[2-oxo-2-(1,2,3,4- | B | B | B |
| | tetrahydronaphthalen-1- | | | |
| | ylamino)ethyl]benzamide | | | |
| 51 | N-[2-oxo-2-(1,2,3,4- | A | A | B |
| | tetrahydronaphthalen-1- | | | |
| | ylamino)ethyl]-4- | | | |
| | [(phenylmethyl)oxy]benzamide | | | |
| 52 | 4′-ethyl-N-{2-oxo-2- | D | D | D |
| | [(phenylmethyl)amino]ethyl}bi- | | | |
| | phenyl-4-carboxamide | | | |
| 53 | N-[2-(cyclohexylamino)-2-oxoethyl]- | D | D | D |
| | 4′-ethylbiphenyl-4-caroxamide | | | |
| 54 | N-(2-{[(3,4- | D | D | D |
| | dichlorophenyl)methyl]amino}-2- | | | |
| | oxoethyl)-4′-ethylbiphenyl-4- | | | |
| | carboxamide | | | |
| 55 | N˜2˜- | D | D | D |
| | [{[cyclopentyl(phenyl)acetyl]amino} | | | |
| | (pyridin-3-yl)acetyl]-N-(1,2,3,4- | | | |
| | tetrahydronaphthalen-1- | | | |
| | yl)glycinamide | | | |
| 56 | 4-hydroxy-N-[2-oxo-2-(1,2,3,4- | B | B | C |
| | tetrahydronaphthaten-1- | | | |
| | ylamino)ethyl]benzamide | | | |
| 57 | N-[2-(1,3-dihydro-2H-isoindol-2-yl)- | D | D | D |
| | 2-oxoethyl]-4′-ethylbiphenyl-4- | | | |
| | carboxamide | | | |
| 58 | 4-morpholin-4-yl-N-[2-oxo-2- | D | C | D |
| | (1,2,3,4-tetrahydronaphthalen-1- | | | |
| | ylamino)ethyl]benzamide | | | |
| 59 | 5,6-bis(methyloxy)-N-[2-oxo-2- | C | B | C |
| | (1,2,3,4-tetrahydronaphthalen-1- | | | |
| | ylamino)ethyl]-1H-indole-2- | | | |
| | carboxamide | | | |
| 60 | N-[2-(3,4-dihydroisoquinolin-2(1H)- | D | D | D |
| | yl)-2-oxoethyl]-4′-ethylbiphenyl-4- | | | |
| | carboxamide | | | |
| 61 | 4′-ethyl-N-{2-oxo-2-[(1S)-1,2,3,4- | A | A | A |
| | tetrahydronaphthalen-1- | | | |
| | ylamino]ethyl}biphenyl-4- | | | |
| | carboxamide | | | |
| 62 | 2-amino-N˜4˜-(2,3-dihydro-1,4- | D | D | D |
| | benzodioxin-2-ylmethyl)-N˜1˜-[3- | | | |
| | (ethyloxy)propyl]benzene-1,4- | | | |
| | dicarboxamide | | | |
| 63 | N-{3-[(2-{[6-(methyloxy)-1,3- | A | A | A |
| | benzothiazol-2-yl]amino}-2- | | | |
| | oxoethyl)amino]-3-oxo-1- | | | |
| | phenylpropyl}benzamide | | | |
| 64 | 4′-ethyl-N-(2-{[6-(methyloxy)-1,3- | D | D | D |
| | benzothiazol-2-yl]amino}-2- | | | |
| | oxoethyl)biphenyl-4-carboxamide | | | |
| 65 | N-{2-[(2,3-dihydro-1,4-benzodioxin- | D | D | D |
| | 2-ylmethyl)amino]-2-oxoethyl}-4′- | | | |
| | ethylbiphenyl-4-carboxamide | | | |
| 66 | 4′-ethyl-N-methyl-N-[2-oxo-2- | D | D | D |
| | (1,2,3,4-tetrahydronaphthalen-1- | | | |
| | ylamino)ethyl]biphenyl-4- | | | |
| | carboxamide | | | |
| 67 | 4′-ethyl-N-[2-(naphthalen-1- | A | A | B |
| | ylamino)-2-oxoethyl]biphenyl-4- | | | |
| | carboxamide | | | |
| 68 | 4′-ethyl-N-[2-oxo-2-(4- | D | D | D |
| | phenylpiperazin-1-yl)ethyl]biphenyl- | | | |
| | 4-carboxamide | | | |
| 69 | N˜2˜-(biphenyl-4-ylmethyl)-N- | D | D | D |
| | (1,2,3,4-tetrahydronaphthalen-1- | | | |
| | yl)glycinamide | | | |
| 70 | 4-(1H-imidazol-1-yl)-N-[2-oxo-2- | C | B | C |
| | (1,2,3,4-tetrahydronaphthalen-1- | | | |
| | ylamino)ethyl]benzamide | | | |
| 71 | 4-(1,3-oxazol-5-yl)-N-[2-oxo-2- | B | A | B |
| | (1,2,3,4-tetrahydronaphthalen-1- | | | |
| | ylamino)ethyl]benzamide | | | |
| 72 | N˜2˜-{[(2-biphenyl-4- | D | D | D |
| | ylethyl)amino]carbonyl}-N-(1,2,3,4- | | | |
| | tetrahydronaphthalen-1- | | | |
| | yl)glycinamide | | | |
| 73 | N˜2˜-[(biphenyl-4- | D | B | D |
| | ylamino)carbonyl]-N-(1,2,3,4- | | | |
| | tetrahydronaphthalen-1- | | | |
| | yl)glycinamide | | | |
| 74 | 4′-ethyl-N-(2-{[(2- | B | B | B |
| | methylphenyl)methyl]amino}-2- | | | |
| | oxoethyl)biphenyl-4-carboxamide | | | |
| 75 | 4′-ethyl-N-{2-oxo-2-[(2- | D | D | D |
| | phenylethyl)amino]ethyl}biphenyl-4- | | | |
| | carboxamide | | | |
| 76 | 4′ethyl-N-{2-oxo-2-[(1R)-1,2,3,4- | D | D | D |
| | tetrahydronaphthalen-1- | | | |
| | ylamino]ethyl}biphenyl-4- | | | |
| | carboxamide | | | |
| 77 | N-[2-oxo-2-(1,2,3,4- | C | B | C |
| | tetrahydronaphthalen-1- | | | |
| | ylamino)ethyl]-4-piperidin-1- | | | |
| | ylbenzamide | | | |
| 78 | 4′-ethyl-N-{2-oxo-2-[(1- | C | B | D |
| | phenylethyl)amino]ethyl}biphenyl-4- | | | |
| | carboxamide | | | |
| 79 | 4′-ethyl-N-[2-oxo-2-({[2- | B | B | D |
| | (trifluoromethyl)phenyl]meth- | | | |
| | yl}amino)ethyl]biphenyl- | | | |
| | 4-carboxamide | | | |
| 80 | N-[2-oxo-2-(1,2,3,4- | C | B | C |
| | tetrahydronaphthalen-1- | | | |
| | ylamino)ethyl]quinoline-6- | | | |
| | carboxamide | | | |
| 81 | N-(3-oxo-3-{[2-oxo-2-(1,2,3,4- | D | D | D |
| | tetrahydronaphthalen-1- | | | |
| | ylamino)ethyl]amino}-1- | | | |
| | phenylpropyl)benzamide | | | |
| 82 | N-[2-oxo-2-(1,2,3,4- | D | C | D |
| | tetrahydronaphthalen-1- | | | |
| | ylamino)ethyl]-5,6,7,8- | | | |
| | tetrahydronaphthalene-2- | | | |
| | carboxamide | | | |
| 83 | 4′-ethyl-N-{2-oxo-2-[(1- | C | B | D |
| | phenylpropyl)amino]ethyl}biphenyl- | | | |
| | 4-carboxamide | | | |
| 84 | 3-(acetylamino)-N-(2-{[6- | D | D | D |
| | (methyloxy)-1,3-benzothiazol-2- | | | |
| | yl]amino}-2-oxoethyl)-3- | | | |
| | phenylpropanamide | | | |
| 85 | N-(phenylcarbonyl)-beta-alanyl-N- | D | D | D |
| | [6-(methyloxy)-1,3-benzothiazol-2- | | | |
| | yl]glycinamide | | | |
| 86 | N-[2-oxo-2-(5,6,7,8- | D | D | D |
| | tetrahydronaphthalen-1- | | | |
| | ylamino)ethyl]quinoline-6- | | | |
| | carboxamide | | | |
| 87 | N-[2-oxo-2-(5,6,7,8- | D | D | D |
| | tetrahydronaphthalen-1- | | | |
| | ylamino)ethyl]quinoline-3- | | | |
| | carboxamide | | | |
| 88 | N-[2-oxo-2-(1,2,3,4- | D | D | D |
| | tetrahydronaphthalen-1- | | | |
| | ylamino)ethyl]-3-piperidin-1- | | | |
| | ylpropanamide | | | |
| 89 | N-[2-oxo-2-(5,6,7,8- | D | D | D |
| | tetrahydronaphthalen-1- | | | |
| | ylamino)ethyl]-4-piperidin-1- | | | |
| | ylbenzamide | | | |
| 90 | N-(3-{[2-(1,3-benzothiazol-2- | B | A | B |
| | ylamino)-2-oxoethyl]amino}-3-oxo- | | | |
| | 1-phenylpropyl)benzamide | | | |
| 91 | N-[2-oxo-2-(1,2,3,4- | D | D | D |
| | tetrahydronaphthalen-1- | | | |
| | ylamino)ethyl]-4-phenylpiperazine- | | | |
| | 1-carboxamide | | | |
| 92 | N-(3-oxo-3-{[2-oxo-2-(1,3-thiazol-2- | C | B | C |
| | ylamino)ethyl]amino}-1- | | | |
| | phenylpropyl)benzamide | | | |
| 93 | N-[3-({2-[(4-methyl-1,3-thiazol-2- | D | D | D |
| | yl)amino]-2-oxoethyl}amino)-3-oxo- | | | |
| | 1-phenylpropyl]benzamide | | | |
| 94 | N-{3-[(2-{[5- | A | A | B |
| | (methyloxy)[1,3]thiazolo[5,4- | | | |
| | b]pyridin-2-yl]amino}-2- | | | |
| | oxoethyl)amino]-3-oxo-1- | | | |
| | phenylpropyl}benzamide | | | |
| 95 | 1,1-dimethylethyl 4-({N-[(4′- | D | C | D |
| | ethylbiphenyl-4- | | | |
| | yl)carbonyl]glycyl}amino)-3,4- | | | |
| | dihydroisoquinoline-2(1H)- | | | |
| | carboxylate | | | |
| 96 | 4′-ethyl-N-[2-oxo-2-(1,2,3,4- | C | C | C |
| | tetrahydroisoquinolin-4- | | | |
| | ylamino)ethyl]biphenyl-4- | | | |
| | carboxamide | | | |
| 97 | N-{3-[(6-hydroxy-1,3-benzothiazol- | D | D | D |
| | 2-yl)amino]-3-oxo-1- | | | |
| | phenylpropyl}benzamide | | | |
| 98 | N-[3-({2-[(6-hydroxy-1,3- | B | A | B |
| | benzothiazol-2-yl)amino]-2- | | | |
| | oxoethyl}amino)-3-oxo-1- | | | |
| | phenylpropyl]benzamide | | | |
| 99 | 2-[(5-bromopyridin-2-yl)amino]-2- | | | |
| | oxoethyl{[2-(naphthalen-1- | | | |
| | ylamino)-2-oxoethyl]thio}acetate | | | |
| 100 | 2-[(5-chloropyridin-2-yl)amino]-2- | | | |
| | oxoethyl(1,3-benzoxazol-2- | | | |
| | ylthio)acetate | | | |
| 101 | 4-{[(5,6-dimethyl-4-oxothieno[2,3- | | | |
| | d]pyrimidin-3(4H)-yl)acetyl]amino}- | | | |
| | N-[6-(methyloxy)-1,3-benzothiazol- | | | |
| | 2-yl]butanamide | | | |

What is claimed is:

1. A compound according to Formula I, 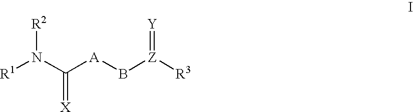
or a pharmaceutically acceptable salt, hydrate, or prodrug thereof, wherein,
R¹ is selected from optionally substituted C₁₋₁₀alkyl, optionally substituted aryl, optionally substituted aryl-C₁₋₁₀alkyl, optionally substituted heterocyclyl, and optionally substituted heterocyclyl-C₁₋₁₀alkyl;
R² is selected from —H and optionally substituted C₁₋₆alkyl;
or R¹ and R² combine to form an optionally substituted three- to seven-membered heteroalicyclic;
A is a C₁₋₃alkylene optionally substituted with one to four of R⁶;
B is selected from —O—, —N(R⁴)—, —S(O)₀₋₂— and —N(CH₂)₂N(CH₂)₂—S(O)₀₋₂—;
X is selected from ═O, ═S, and ═NR⁷;
Y is selected from ═O, ═S, and ═NR⁷;
Z is C; or
Z=Y is either absent or —CH₂—;
R³ is selected from —H, halogen, trihalomethyl, —OR⁵, —N(R⁵)R⁵, —N(R⁵)SO₂R⁵, —N(R⁵)C(O)R⁵, —NCO₂R⁵, optionally substituted alkoxy, optionally substituted alkyl, optionally substituted aryl, optionally substituted arylalkyl, optionally substituted heterocyclyl, and optionally substituted heterocyclylalkyl;
R⁴ is selected from —H and optionally substituted C₁₋₆alkyl; or
R⁴ and one of R⁶, together with the atoms to which they are attached, combine to form an optionally substituted five- to seven-membered non-aromatic ring;
each R⁵ is independently selected from —H, optionally substituted lower alkyl, optionally substituted aryl, optionally substituted lower arylalkyl, optionally substituted heterocyclyl, and optionally substituted lower heterocyclylalkyl;
two of R⁵, together with the atom or respective atoms to which they are attached, can combine to form an optionally substituted five- to seven-membered heterocyclic;
each R⁶ is independently selected from —H, halogen, trihalomethyl, —CN, —NO₂, —NH₂, —OR⁵, —NR⁵R⁵, —S(O)₀₋₂R⁵, —SO₂NR⁵R⁵, —CO₂R⁵, —C(O)NR⁵R⁵, —N(R⁵)SO₂R⁵. —N(R⁵)C(O)R⁵, —N(R⁵)CO₂R⁵, —C(O)R⁵, optionally substituted lower alkyl, optionally substituted aryl, optionally substituted heterocyclyl, optionally substituted lower heterocyclylalkyl, and optionally substituted lower arylalkyl;
two of R6, together with the atom or atoms to which they are attached, can combine to form a three to seven-membered non-aromatic ring; and
each R⁷ is independently selected from —H, —NO₂, —NH₂, —N(R⁵)R⁵, —CN, —OR⁵, optionally substituted lower alkyl, optionally substituted heteroalicyclylalkyl, optionally substituted aryl, optionally substituted arylalkyl and optionally substituted heteroalicyclic;
provided the compound is not one of: 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-Dimethyl-4-oxo-4H-thieno[2,3-d]pyrimidin-3-yl)-N-[(6-methoxy-benzothiazol-2-ylcarbamoyl)-methyl]-acetamide, (Naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide.

2. The compound of claim 1, according to one of the following formulae: 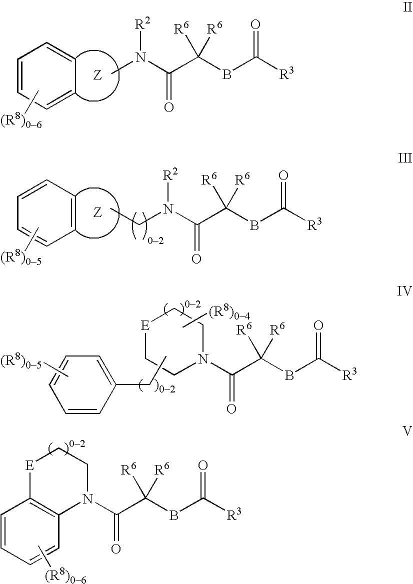
wherein R², R³, R⁶, and B are as defined above; Z is a five- to seven-membered ring; each R⁸ is independently selected from —H, halogen, trihalomethyl, —CN, —NO₂, —NH₂, —OR⁵, —NR⁵R⁵, —S(O)₀₋₂R⁵, —SO₂NR⁵R⁵, —CO₂R⁵, —C(O)NR⁵R⁵, —N(R⁵)SO₂R⁵, —N(R⁵)C(O)R⁵, —N(R⁵)CO₂R⁵, —C(O)R⁵, optionally substituted lower alkyl, optionally substituted aryl, optionally substituted heterocyclylalkyl, and optionally substituted arylalkyl; two of R⁸, together with the atom or atoms to which they are attached, can combine to form a three- to seven-membered ring; and E is selected from —O—, —N(R⁹)—, —CH₂—, and —S(O)₀₋₂—, where R⁹ is selected from —H, trihalomethyl, —S(O)₀₋₂R⁵, —SO₂NR⁵R⁵, —CO₂R⁵, —C(O)NR⁵R⁵, —C(O)R⁵, optionally substituted lower alkyl, optionally substituted aryl, optionally substituted heterocyclylalkyl, and optionally substituted arylalkyl.

3. The compound according to claim 2, wherein B is selected from —O—, —N(R⁴)—, and —S(O)₀₋₁—.

4. The compound according to claim 3, according to either formula II or III.

5. The compound according to claim 4, wherein R⁴ is —H or C₁₋₆alkyl.

6. The compound according to claim 5, wherein R² is —H or C₁₋₆alkyl.

7. The compound of claim 6, according to formula IIa.

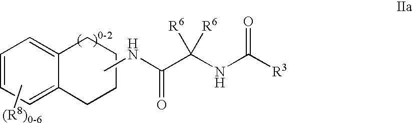

8. The compound according to claim 7, wherein each R⁶ is independently selected from —H, trihalomethyl, optionally substituted lower alkyl, optionally substituted aryl, optionally substituted heterocyclyl, optionally substituted heterocyclylalkyl, and optionally substituted arylalkyl.

9. The compound according to claim 8, wherein one of R⁶ is —H, and the other R⁶ is selected from trihalomethyl, optionally substituted lower alkyl, optionally substituted aryl, optionally substituted heterocyclyl, optionally substituted heterocyclylalkyl, and optionally substituted arylalkyl.

10. The compound according to claim 9, wherein R³ is selected from optionally substituted alkoxy, optionally substituted lower alkyl, optionally substituted aryl, optionally substituted lower arylalkyl, optionally substituted heterocyclyl, and optionally substituted lower heterocyclylalkyl.

11. The compound according to claim 10, wherein R³ is selected from lower alkyl substituted with an optionally substituted aryloxy or an optionally substituted heteroaryloxy, optionally substituted aryl, optionally substituted lower arylalkyl, optionally substituted heteroaryl, and optionally substituted lower heteroarylalkyl.

12. The compound according to claim 11, wherein R³ is an aryl, said aryl substituted with at least one of an optionally substituted aryl and an optionally substituted heteroaryl.

13. The compound according to claim 12, wherein R³ is an optionally substituted bis-phenyl.

14. The compound according to claim 13, wherein R3 comprises an optionally substituted phenylene, wherein the point of attachment of R3 according to formula IIa, and an optionally substituted phenyl bear a para relationship to one another about said optionally substituted phenylene.

15. A compound for modulating at least one kinase activity according to formula VI, 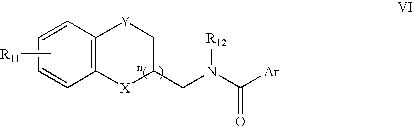
or a pharmaceutically acceptable salt, hydrate, or prodrug thereof, wherein, each of R¹¹ and R¹² is independently selected from —H, halogen, trihalomethyl, —CN, —NO₂, —NR¹⁴R¹⁴, —S(O)₀₋₂R¹⁴, —SO₂NR¹⁴R¹⁴, —CO₂R¹⁴, —C(O)NR¹⁴R¹⁴, —N(R¹⁴)SO₂R¹⁴, —N(R¹⁴)C(O)R¹⁴, —N(R¹⁴)CO₂R¹⁴, —OR¹⁴, —C(O)R¹⁴, optionally substituted lower alkyl, optionally substituted alkoxy, optionally substituted aryl, optionally substituted heterocyclyl, optionally substituted heterocyclylalkyl, and optionally substituted arylalkyl;
R¹⁴ is selected from —H, optionally substituted lower alkyl, optionally substituted aryl, optionally substituted lower arylalkyl, optionally substituted heterocyclyl, and optionally substituted lower heterocyclylalkyl;
each of X and Y is independently selected from —O—, —N(R¹⁴)—, and —S(O)₀₋₂—;
n is selected from an integer from 0-2;
Ar is an optionally substituted aryl that may be substituted with up to three R¹¹, wherein two adjacent R¹¹'s, together with the annular atoms to which they are attached, can form a five- to seven-membered ring containing up to three heteroatoms and optionally substituted with up to three of R¹⁵;
each R¹⁵ is independently selected from —H, halo, trihalomethyl, —CN, —NO₂, —OR¹⁶, —N(R¹⁶)R¹⁶, —S(O)₀₋₂R¹⁶, —SO₂N(R¹⁶)R¹⁶, —CO₂R¹⁶, —C(═O)N(R¹⁶)R¹⁶, —C(═NR¹⁷)N(R¹⁶)R¹⁶, —C(═NR¹⁷)R¹⁶, —N(R¹⁶)SO₂R¹⁶, —N(R¹⁶)C(O)R¹⁶, —NCO₂R¹⁶, —C(═O)R¹⁶, optionally substituted alkoxy, optionally substituted lower alkyl, optionally substituted aryl, optionally substituted lower arylalkyl, optionally substituted heterocyclyl, and optionally substituted lower heterocyclylalkyl;
R¹⁶ is selected from —H, optionally substituted lower alkyl, optionally substituted aryl, optionally substituted lower arylalkyl, optionally substituted heterocyclyl, and optionally substituted lower heterocyclylalkyl; and
R¹⁷ is selected from —H, —CN, —NO₂, —OR¹⁶, —S(O)₀₋₂R¹⁶, —CO₂R¹⁶, optionally substituted lower alkyl, optionally substituted lower alkenyl, and optionally substituted lower alkynyl.

16. A compound according to claim 15, wherein X is O.

17. A compound according to claim 16, wherein Y is O.

18. A compound according to claim 17, wherein R¹¹ is —H.

19. A compound according to claim 18, wherein R¹² is —H.

20. A compound according to claim 19, wherein n is 1.

21. A compound according to claim 20, wherein Ar is substituted aryl and two adjacent R¹¹'s, together with the annular atoms to which they are attached, form a substituted six-membered ring containing up to three heteroatoms.

22. A compound according to claim 20, wherein Ar is substituted aryl and two adjacent R¹¹'s, together with the annular atoms to which they are attached, form a substituted seven-membered ring containing up to three heteroatoms.

23. The compound according to claim 1 or 15, selected from the following:
TABLE 1

| Entry | Name | Structure |
|---|---|---|
| 1 | N-[2-oxo-2-(1,2,3,4- tetrahydronaphthalen-1- ylamino)ethyl]-4- (pentyloxy)benzamide | 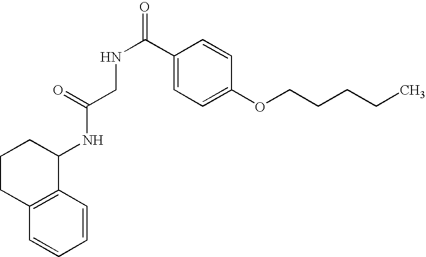 |
| 2 | N˜2˜-[({4- [(phenylmethyl)oxy]phenyl}oxy)acet yl]-N-(1,2,3,4-tetrahydronaphthalen- 1-yl)glycinamide | 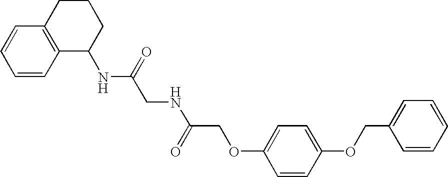 |
| 3 | N˜2˜-{[(4-bromophenyl)oxy]acetyl}- N-(1,2,3,4-tetrahydronaphthalen-1- yl)glycinamide | 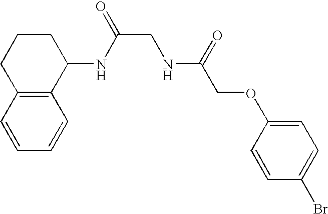 |
| 4 | 4′-ethyl-N-[2-oxo-2-(1,2,3,4- tetrahydronaphthalen-1- ylamino)ethyl]biphenyl-4- carboxamide | 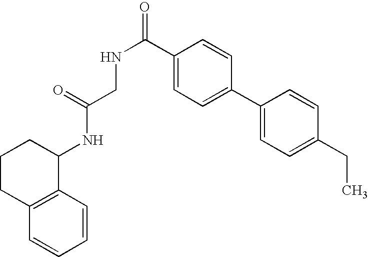 |
| 5 | 4′-ethyl-N-[1-methyl-2-oxo-2- (1,2,3,4-tetrahydronaphthalen-2- ylamino)ethyl]biphenyl-4- carboxamide | 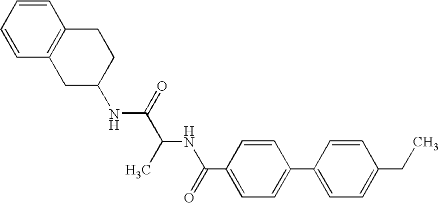 |
| 6 | 4-(hexyloxy)-N-[2-oxo-2-(1,2,3,4- tetrahydronaphthalen-1- ylamino)ethyl]benzamide | 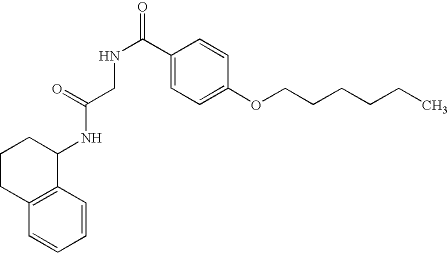 |
| 7 | 2-cyclopentyl-N-[2-oxo-1-pyridin-3- yl-2-(1,2,3,4-tetrahydronaphthalen- 1-ylamino)ethyl]-2-phenylacetamide | 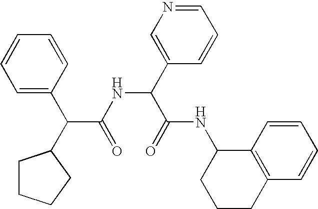 |
| 8 | 4-(heptyloxy)-N-[2-oxo-2-(1,2,3,4- tetrahydronaphthalen-1- ylamino)ethyl]benzamide | 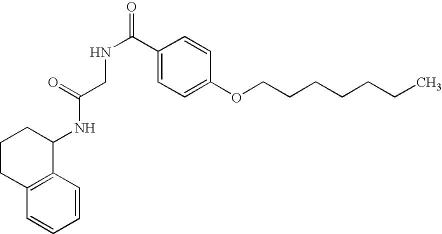 |
| 9 | N-[1-methyl-2-oxo-2-(1,2,3,4- tetrahydronaphthalen-2- ylamino)ethyl]-4- (pentyloxy)benzamide | 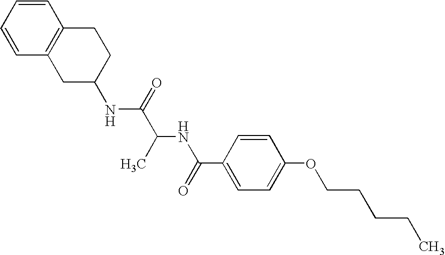 |
| 10 | 4-hexyl-N-[2-oxo-2-(1,2,3,4- tetrahydronaphthalen-1- ylamino)ethyl]benzamide | 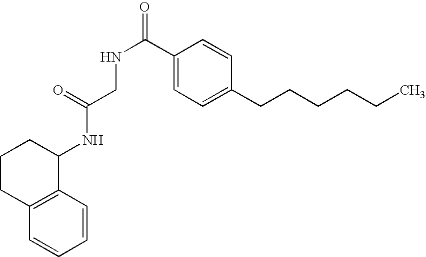 |
| 11 | N-[2-oxo-2-(1,2,3,4- tetrahydronaphthalen-1- ylamino)ethyl]-4-pentylbenzamide | 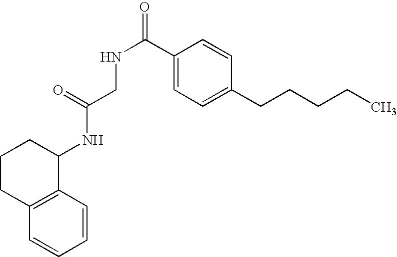 |
| 12 | 4-heptyl-N-[2-oxo-2-(1,2,3,4- tetrahydronaphthalen-1- ylamino)ethyl]benzamide | 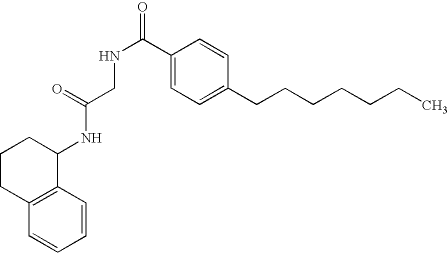 |
| 13 | Nalpha-{[5,6-bis(methyloxy)-1H- indol-2-yl]carbonyl}-N-(1,2,3,4- tetrahydronaphthalen-1- yl)tryptophanamide | 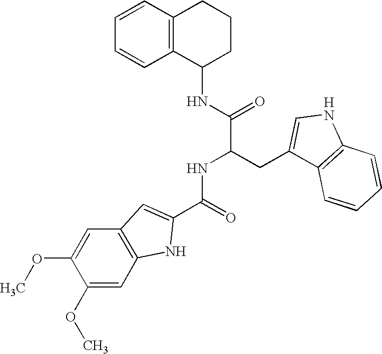 |
| 14 | Nalpha-{[4- (butyloxy)phenyl]carbonyl}-N-(1,2,3,4-tetrahydronaphthalen-1- yl)tryptophanamide | 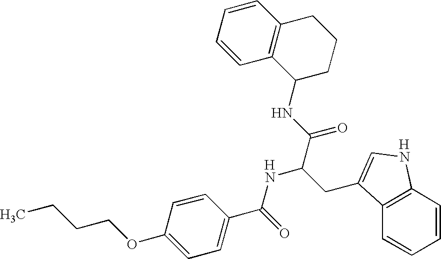 |
| 15 | 5-{(2E)-3-[3,4- bis(methyloxy)phenyl]prop-2-enoyl}- N-[2-(3-chlorophenyl)ethyl]-4,5,6,7- tetrahydro-3H-imidazol[4,5- c]pyridine-6-carboxamide | 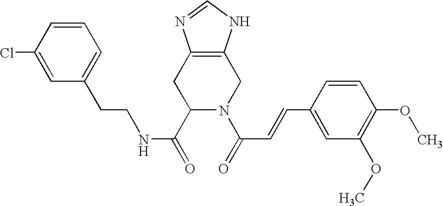 |
| 16 | N-[2-oxo-1-pyridin-3-yl-2-(1,2,3,4- tetrahydronaphthalen-1- ylamino)ethyl]-4-(1H-1,2,4-triazol-1- yl)benzamide | 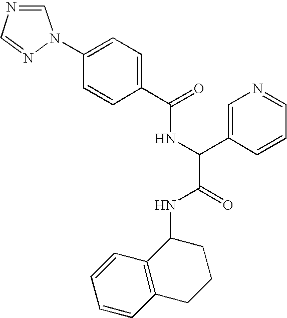 |
| 17 | Nalpha-{(2E)-3-[3,4- bis(methyloxy)phenyl]prop-2-enoyl}- N-(1,2,3,4-tetrahydronaphthalen-1- yl)tryptophanamide | 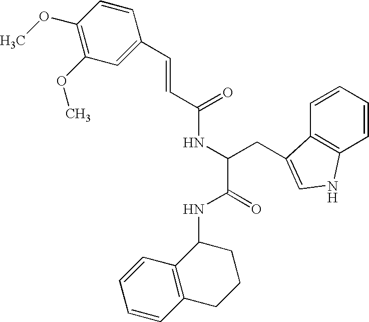 |
| 18 | 4-(butyloxy)-N-[1-methyl-2-oxo-2- (1,2,3,4-tetrahydronaphthalen-2- ylamino)ethyl]benzamide | 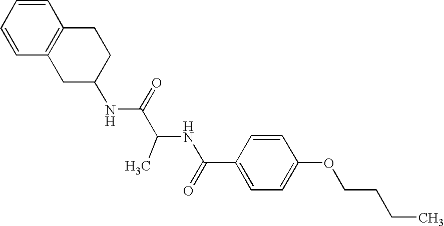 |
| 19 | N-[1-methyl-2-oxo-2-(1,2,3,4- tetrahydronaphthalen-2- ylamino)ethyl]-4- [(phenylmethyl)oxy]benzamide | 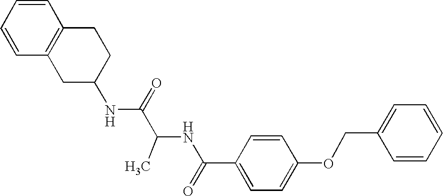 |
| 20 | 3,5-dimethyl-N-{2-oxo-2-(1,2,3,4- tetrahydronaphthalen-1- ylamino)ethyl]-4- [(phenylmethyl)oxy]benzamide | 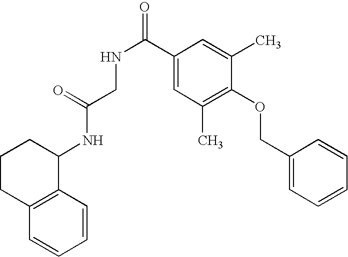 |
| 21 | N˜2˜-({[4- (butyloxy)phenyl]amino}carbonyl)- N-(2-phenylethyl)-O- (phenylmethyl)serinamide | 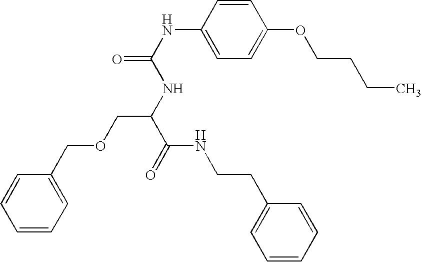 |
| 22 | (2S)-N˜-1˜-(4-butylphenyl)-N˜2˜- [(3,4- dichlorophenyl)methyl]pyrrolidine- 1,2-dicarboxamide | 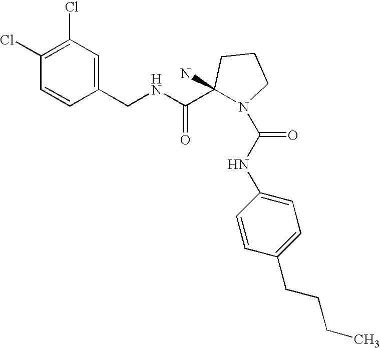 |
| 23 | N-[6-(methyloxy)-1,3-benzothiazol- 2-yl]-4-{[(4-oxo-5,6,7,8- tetrahydro[1]benzothieno[2,3- d]pyrimidin-3(4H)- yl)acetyl]amino}butanamide | 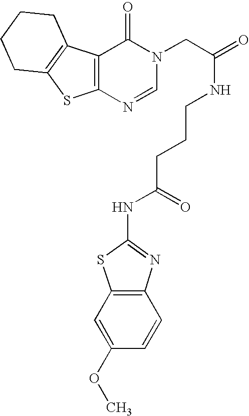 |
| 24 | (2S)-N˜2˜-[(3,4- dichlorophenyl)methyl]-N˜1˜-[4-(1- methylethyl)phenyl]pyrrolidine-1,2- dicarboxamide | 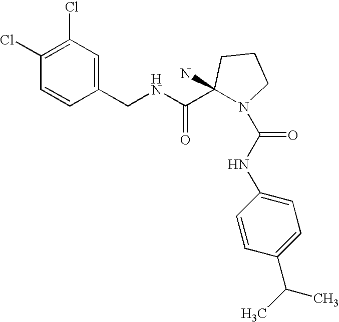 |
| 25 | N˜2˜-{[(4-bromophenyl)oxy]acetyl}- N-[(3,4- dichlorophenyl)methyl]glycinamide | 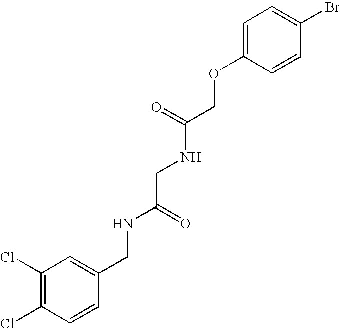 |
| 26 | N-[2-oxo-1-pyridin-3-yl-2-(1,2,3,4- tetrahydronaphthalen-1- ylamino)ethyl]-4-[(E)- phenyldiazenyl]benzamide | 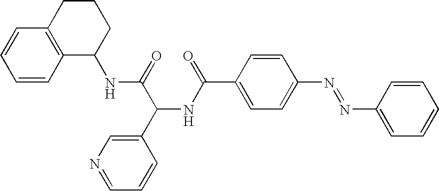 |
| 27 | 4-(butyloxy)-N-[2-oxo-2-(1,2,3,4- tetrahydronaphthalen-1- ylamino)ethyl]benzamide | 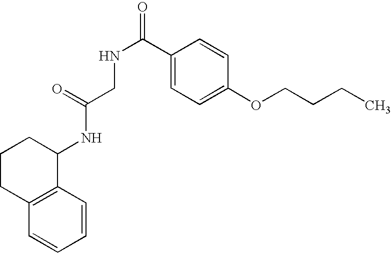 |
| 28 | N˜2˜-({[3- (methyloxy)phenyl]oxy}acetyl)-N- (1,2,3,4-tetrahydronaphthalen-1- yl)glycinamide | 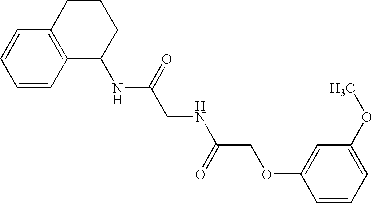 |
| 29 | 4-butyl-N-[2-oxo-2-(1,2,3,4- tetrahydronaphthalen-1- ylamino)ethyl]benzamide | 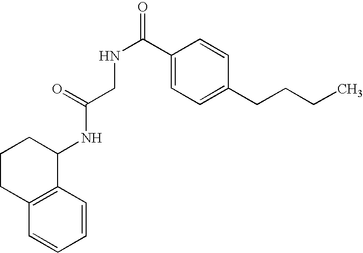 |
| 30 | N˜2˜-{(2E)-3-[3,4- bis(methyloxy)phenyl]prop-2-enoyl}- N-(diphenylmethyl)-O- (phenylmethyl)serinamide | 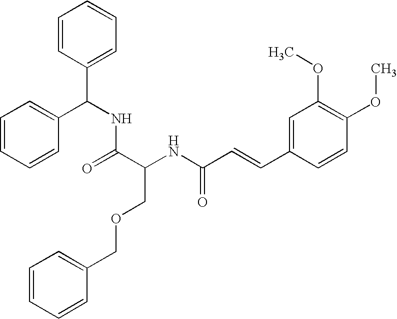 |
| 31 | N-(1,2,3,4-tetrahydronaphthalen-1- yl)-N˜2˜-[({4- [(trifluoromethyl)oxy]phenyl}oxy)ace tyl]glycinamide | 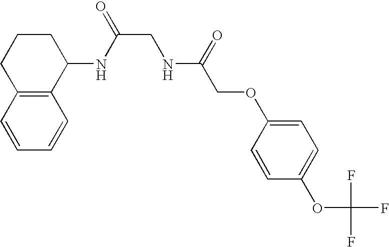 |
| 32 | N-{3-methyl-1-[(1,2,3,4- tetrahydronaphthalen-1- ylamino)carbonyl]butyl}biphenyl-4- carboxamide | 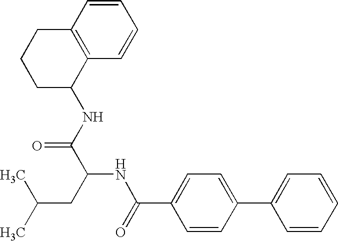 |
| 33 | N-˜2˜-({[4-(1,1- dimethylethyl)phenyl]oxy}acetyl)-N- (1,2,3,4-tetrahydronaphthalen-1- yl)glycinamide | 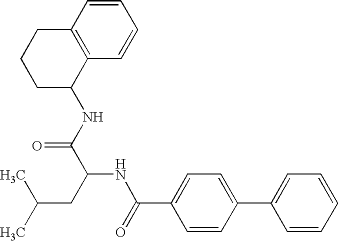 |
| 34 | N˜2˜-{[(4-chlorophenyl)oxy]acetyl}- N-(1,2,3,4-tetrahydronaphthalen-1- yl)glycinamide | 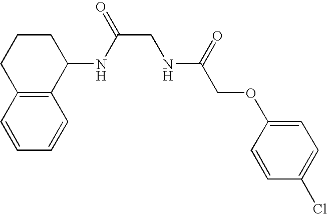 |
| 35 | N˜2˜-{[4- (phenyl]carbonyl}-N- (1,2,3,4-tetrahydronaphthalen-1- yl)leucinamide | 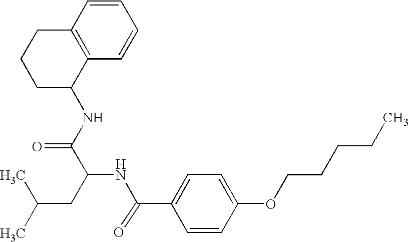 |
| 36 | 4-(hexyloxy)-N-[1-methyl-2-oxo-2- (1,2,3,4-tetrahydronaphthalen-2- ylamino)ethyl]benzamide | 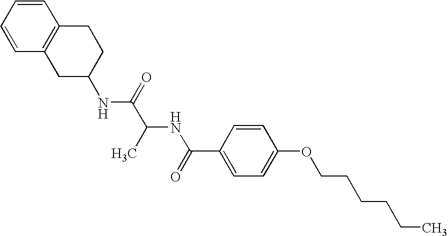 |
| 37 | 2-{[6-(methyloxy)-1,3-benzothiazol- 2-yl]amino}-2-oxoethyl 3-phenyl-3- [(phenylcarbonyl)amino]propanoate | 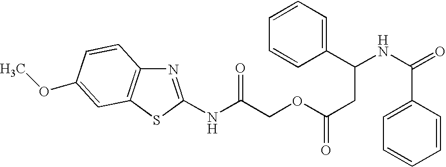 |
| 38 | N-[(3,4-dichlorophenyl)methyl]-2- {[4-(4-pyridin-2-ylpyrimidin-2- yl)phenyl]oxy]acetamide | 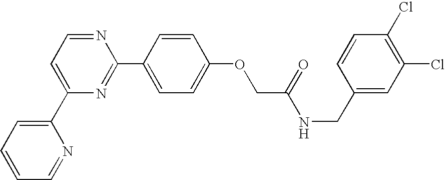 |
| 39 | N-(2,3-dihydro-1,4-benzodioxin-2- ylmethyl)-3-[3-(ethyloxy)propyl]-4- oxo-2-thioxo-1,2,3,4- tetrahydroquinazoline-7- carboxamide | 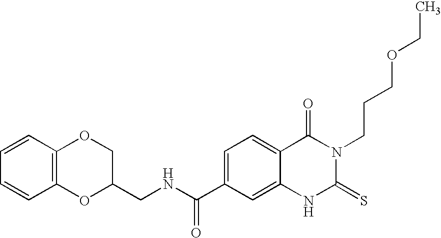 |
| 40 | N˜2˜-[(5,6-dimethyl-4- oxothieno[2,3-d]pyrimidin-3(4H)- yl)acetyl]-N-[6-(methyloxy)-1,3- benzothiazol-2-yl]glycinamide | 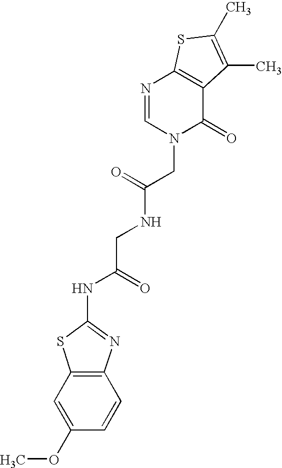 |
| 41 | 2-{4-[(2-naphthalen-1- ylethyl)sulfonyl]piperazin-1-yl)-N- pyridin-2-ylacetamide | 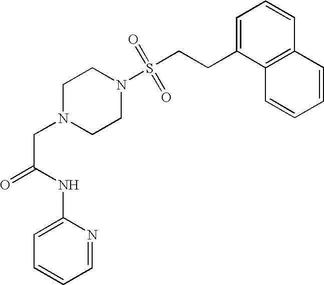 |
| 42 | N-(2,3-dihydro-1,4-benzodioxin-2- ylmethyl)-11-oxo-10,11-dihydro-5H- dibenzo[b,e][1,4]diazepine-8- carboxamide | 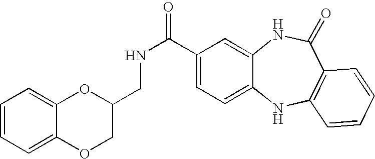 |
| 43 | N-[2-oxo-2-(1,2,3,4- tetrahydronaphthalen-1- ylamino)ethyl]biphenyl-4- carboxamide | 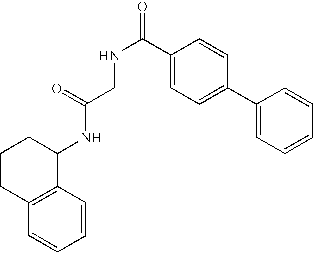 |
| 44 | N-[2-oxo-2-(1,2,3,4- tetrahydronaphthalen-1- ylamino)ethyl]naphthalene-2- carboxamide | 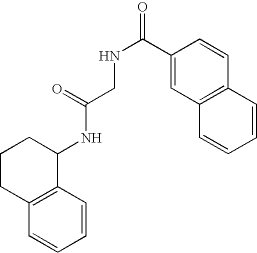 |
| 45 | N-[2-oxo-2-(1,2,3,4- tetrahydronaphthalen-1- ylamino)ethyl]-4- [(trifluoromethyl)oxy]benzamide | 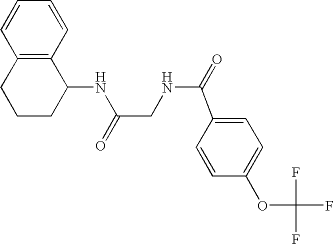 |
| 46 | N-[2-oxo-2-(1,2,3,4- tetrahydronaphthalen-1- ylamino)ethyl]quinoline-3- carboxamide | 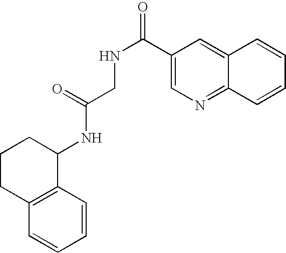 |
| 47 | N˜2˜-({[3,5- bis(trifluoromethyl)phenyl]amino}car bonyl)-N-(1,2,3,4- tetrahydronaphthalen-1- yl)glycinamide | 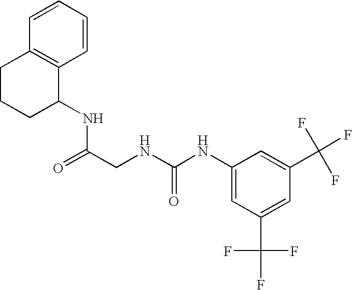 |
| 48 | N˜2˜-{[(3- ethylphenyl)amino]carbonyl}-N- (1,2,3,4-tetrahydronaphthalen-1- yl)glycinamide | 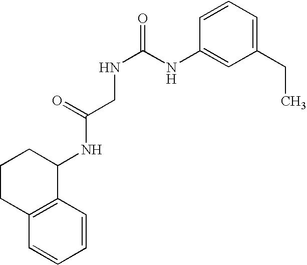 |
| 49 | N-[2-oxo-2-(1,2,3,4- tetrahydronaphthalen-1- ylamino)ethyl]-4- (phenyloxy)benzamide | 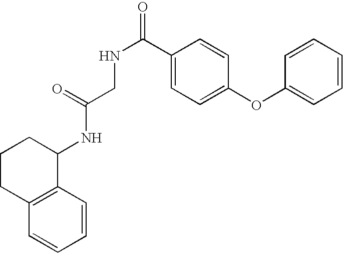 |
| 50 | 4-cyclohexyl-N-[2-oxo-2-(1,2,3,4- tetrahydronaphthalen-1- ylamino)ethyl]benzamide | 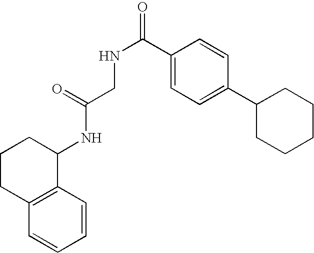 |
| 51 | N-[2-oxo-2-(1,2,3,4- tetrahydronaphthalen-1- ylamino)ethyl]-4- [(phenylmethyl)oxy]benzamide | 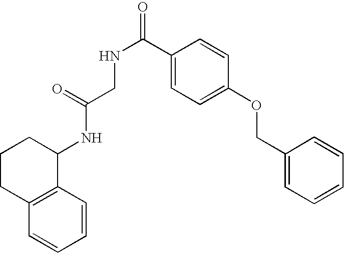 |
| 52 | 4-ethyl-N-{2-oxo-2- [(phenylmethyl)amino]ethyl}biphenyl -4-carboxamide | 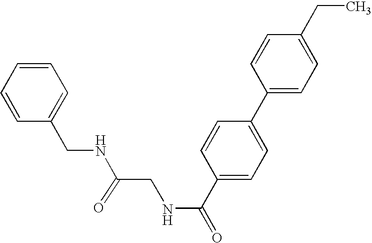 |
| 53 | N-[2-(cyclohexylamino)-2-oxoethyl]- 4′-ethylbiphenyl-4-carboxamide | 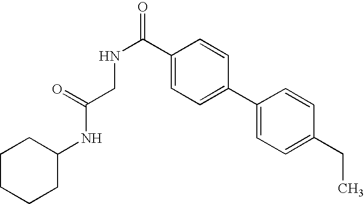 |
| 54 | N-(2-{[(3,4- dichlorophenyl)methyl]amino}-2- oxoethyl)-4′-ethylbiphenyl-4- carboxamide | 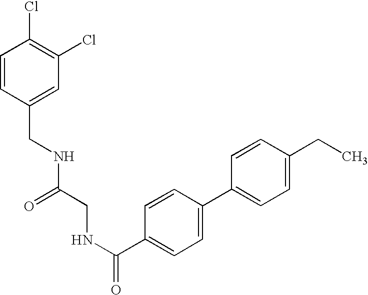 |
| 55 | N˜2˜- [{[cyclopentyl(phenyl)acetyl]amino}(pyridin-3-yl)acetyt]-N-(1,2,3,4- tetrahydronaphthalen-1- yl)glycinamide | 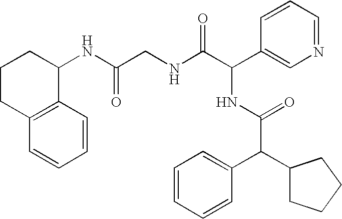 |
| 56 | 4-hydroxy-N-[2-oxo-2-(1,2,3,4- tetrahydronaphthalen-1- ylamino)ethyl]benzamide | 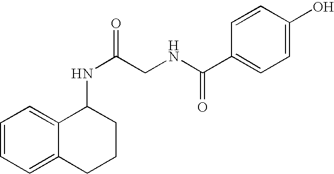 |
| 57 | N-[2-(1,3-dihydro-2H-isoindol-2-yl)- 2-oxoethyl]-4′-ethylbiphenyl-4- carboxamide | 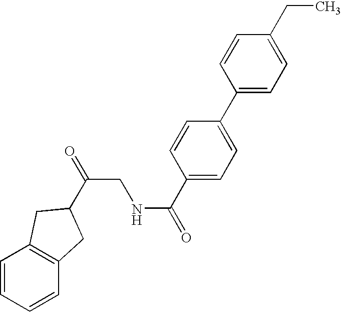 |
| 58 | 4-morpholin-4-yl-N-[2-oxo-2- (1,2,3,4-tetrahydronaphthalen-1- ylamino)ethyl]benzamide | 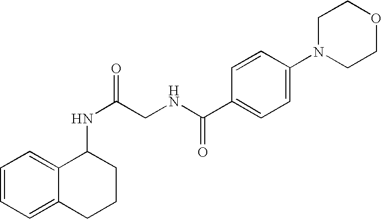 |
| 59 | 5,6-bis(methyloxy)-N-(2-oxo-2- (1,2,3,4-tetrahydronaphthalen-1- ylamino)ethyl]-1H-indole-2- carboxamide | 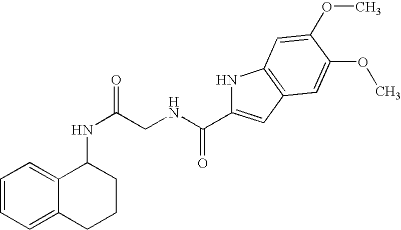 |
| 60 | N-[2-(3,4-dihydroisoquinolin-2(1H)- yl)-2-oxoethyl]-4′-ethylbiphenyl-4- carboxamide | 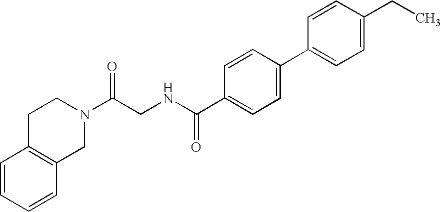 |
| 61 | 4′-ethyl-N-{2-oxo-2-[(1S)-1,2,3,4- tetrahydronaphthalen-1- ylamino]ethyl}biphenyl-4- carboxamide | 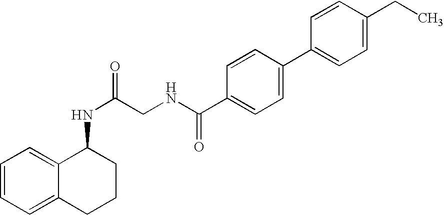 |
| 62 | 2-amino-N-˜4˜-(2,3-dihydro-1,4- benzodioxin-2-ylmethyl)-N˜1˜-[3- (ethyloxy)propyl]benzene-1,4- dicarboxamide | 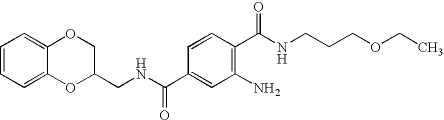 |
| 63 | N-{3-[(2-{[6-(methyloxy)-1,3- benzothiazol-2-yl]amino}-2- oxoethyl)amino]-3-oxo-1- phenylpropyl}benzamide | 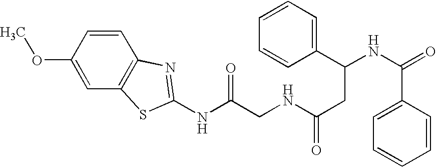 |
| 64 | 4′ethyl-N-(2-{[6-(methyloxy)-1,3- benzothiazol-2-yl]amino}-2- oxoethyl)biphenyl-4-carboxamide | 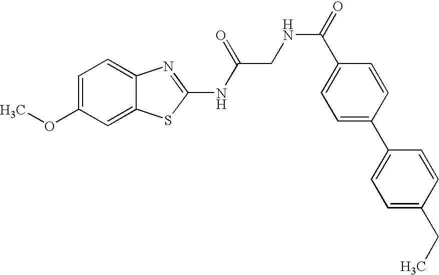 |
| 65 | N-{2-[(2,3-dihydro-1,4-benzodioxin- 2-ylmethyl)amino]-2-oxoethyl}-4′- ethylbiphenyl-4-carboxamide | 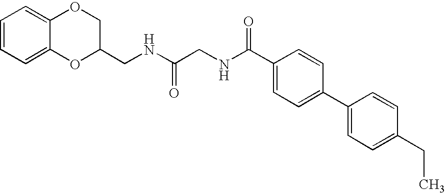 |
| 66 | 4′-ethyl-N-methyl-N-[2-oxo-2- (1,2,3,4-tetrahydronaphthalen-1- ylamino)ethyl]biphenyl-4- carboxamide | 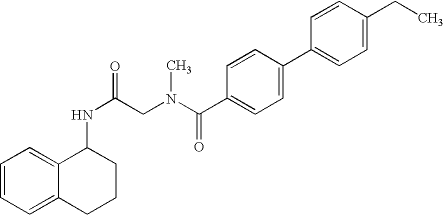 |
| 67 | 4′-ethyl-N-[2-(naphthalen-1- ylamino)-2-oxoethyl]biphenyl-4- carboxamide | 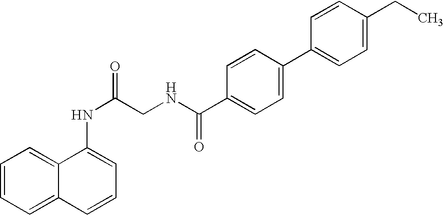 |
| 68 | 4′-ethyl-N-[2-oxo-2-(4- phenylpiperazin-1-yl)ethyl]biphenyl- 4-carboxamide | 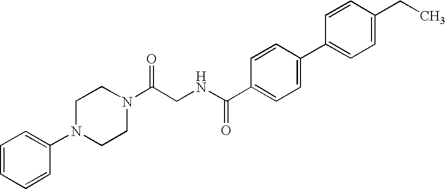 |
| 69 | N˜2˜-(biphenyl-4-ylmethyl)-N- (1,2,3,4-tetrahydronaphthalen-1- yl)glycinamide | 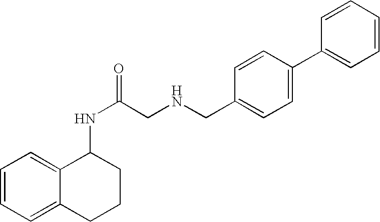 |
| 70 | 4-(1H-imidazol-1-yl)-N-[2-oxo-2- (1,2,3,4-tetrahydronaphthalen-1- ylamino)ethyl]benzamide | 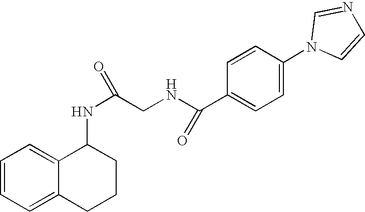 |
| 71 | 4-(1,3-oxazol-5-yl)-N-[2-oxo-2- (1,2,3,4-tetrahydronaphthalen-1- ylamino)ethyl]benzamide | 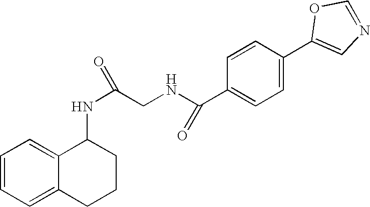 |
| 72 | N˜2˜-{[(2-biphenyl-4- ylethyl)amino]carbonyl}-N-(1,2,3,4- tetrahydronaphthalen-1- yl)glycinamide | 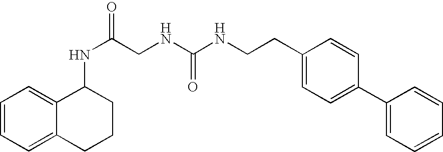 |
| 73 | N˜2˜-[(biphenyl-4- ylamino)carbonyl]-N-(1,2,3,4- tetrahydronaphthalen-1- yl)glycinamide | 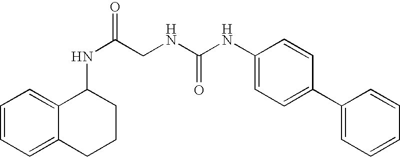 |
| 74 | 4′-ethyl-N-(2-{[(2- methylphenyl)methyl]amino}-2- oxoethyl)biphenyl-4-carboxamide | 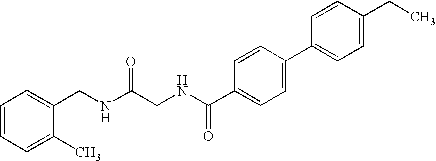 |
| 75 | 4′-ethyl-N-{2-oxo-2-[(2- phenylethyl)amino]ethyl}biphenyl-4- carboxamide | 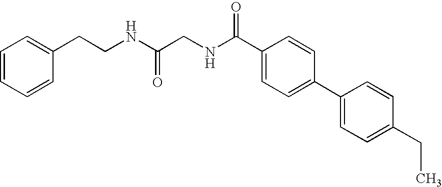 |
| 76 | 4′-ethyl-N-{2-oxo-2-[(1R)-1,2,3,4- tetrahydronaphthalen-1- ylamino]ethyl}biphenyl-4- carboxamide | 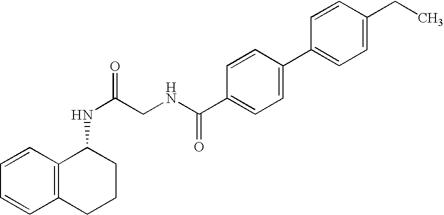 |
| 77 | N-[2-oxo-2-(1,2,3,4- tetrahydronaphthalen-1- ylamino)ethyl]-4-piperidin-1- ylbenzamide | 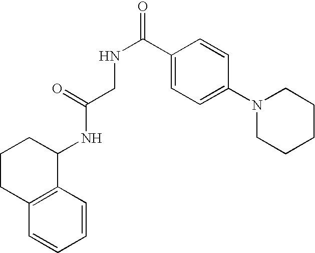 |
| 78 | 4′-ethyl-N-{2-oxo-2-[(1- phenylethyl)amino]ethyl}biphenyl-4- carboxamide | 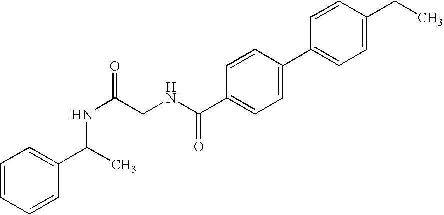 |
| 79 | 4′-ethyl-N-[2-oxo-2-({[2- (trifluoromethyl)phenyl]methyl}amino) ethyl]biphenyl-4-carboxamide | 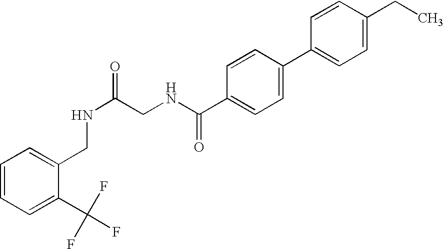 |
| 80 | N-[2-oxo-2-(1,2,3,4- tetrahydronaphthalen-1- ylamino)ethyl]quinoline-6- carboxamide | 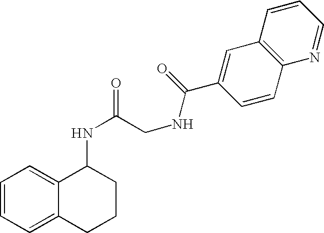 |
| 81 | N-(3-oxo-3-{[2-oxo-2-(1,2,3,4- tetrahydronaphthalen-1- ylamino)ethyl]amino}-1- phenylpropyl)benzamide | 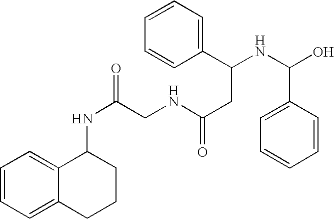 |
| 82 | N-[2-oxo-2-(1,2,3,4- tetrahydronaphthalen-1- ylamino)ethyl]-5,6,7,8- tetrahydronaphthalene-2- carboxamide | 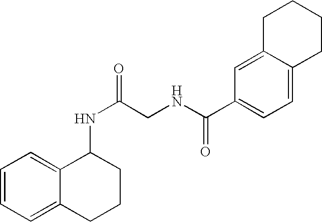 |
| 83 | 4′-ethyl-N-{2-oxo-2-[(1- phenylpropyl)amino]ethyl}biphenyl- 4-carboxamide | 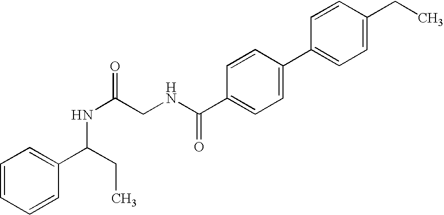 |
| 84 | 3-(acetylamino)-N-(2-{[6- (methyloxy)-1,3-benzothiazol-2- yl]amino}-2-oxoethyl)-3- phenylpropanamide | 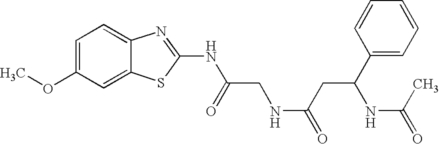 |
| 85 | N-(phenylcarbonyl)-beta-alanyl-N- [6-(methyloxy)-1,3-benzothiazol-2- yl]glycinamide | 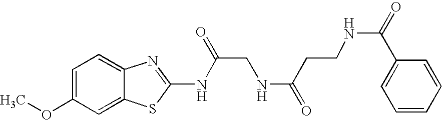 |
| 86 | N-[2-oxo-2-(5,6,7,8- tetrahydronaphthalen-1- ylamino)ethyl]quinoline-6- carboxamide | 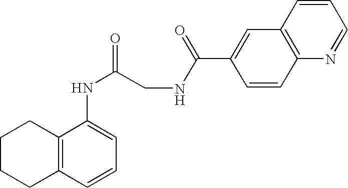 |
| 87 | N-[2-oxo-2-(5,6,7,8- tetrahydronaphthalen-1- ylamino)ethyl]quinoline-3- carboxamide | 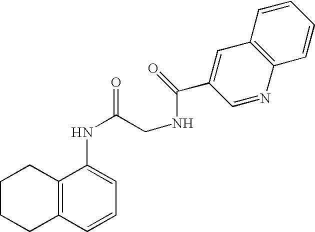 |
| 88 | N-[2-oxo-2-(1,2,3,4- tetrahydronaphthalen-1- ylamino)ethyl]-3-piperidin-1- ylpropanamide | 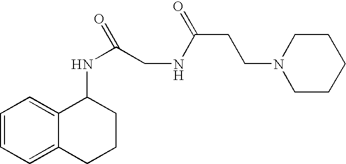 |
| 89 | N-[2-oxo-2-(5,6,7,8- tetrahydronaphthalen-1- ylamino)ethyl]-4-piperidin-1- ylbenzamide | 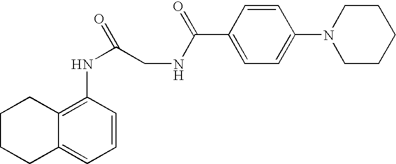 |
| 90 | N-(3-{[2-(1,3-benzothiazol-2- ylamino)-2-oxoethyl]amino}-3-oxo- 1-phenylpropyl)benzamide | 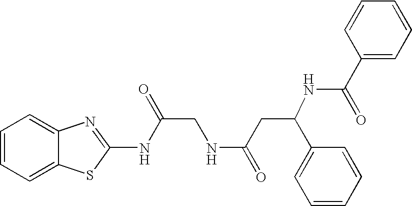 |
| 91 | N-[2-oxo-2-(1,2,3,4- tetrahydronaphthalen-1- ylamino)ethyl]-4-phenylpiperazine 1-carboxamide | 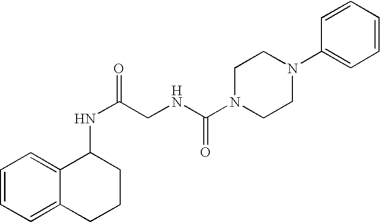 |
| 92 | N-(3-oxo-3-{[2-oxo-2-(1,3-thiazol-2- ylamino)ethyl]amino}-1- phenylpropyl)benzamide | 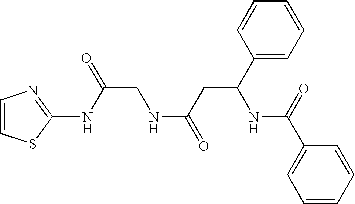 |
| 93 | N-[3-({2-[(4-methyl-1,3-thiazol-2- yl)amino]-2-oxoethyl}amino)-3-oxo- 1-phenylpropyl]benzamide | 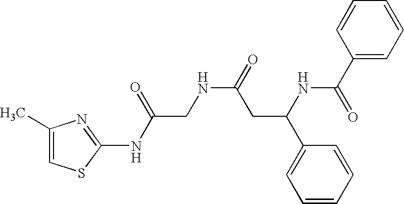 |
| 94 | N-{3-[(2-{[5- (methyloxy)[1,3]thiazolo[5,4- b]pyridin-2-yl]amino}-2- oxoethyl)amino]-3-oxo-1- phenylpropyl}benzamide | 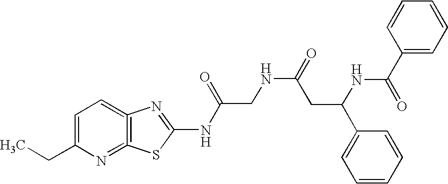 |
| 95 | 1,1-dimethylethyl 4-({N-[(4′- ethylbiphenyl-4- yl)carbonyl]glycyl}amino)-3,4- dihydroisoquinoline-2(1H)- carboxylate | 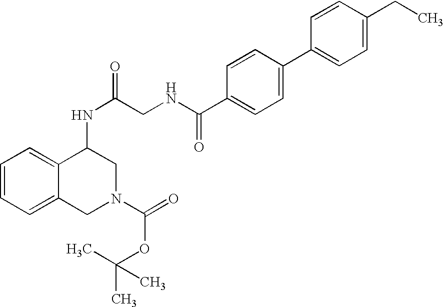 |
| 96 | 4′-ethyl-N-[2-oxo-2-(1,2,3,4- tetrahydroisoquinolin-4- ylamino)ethyl]biphenyl-4- carboxamide | 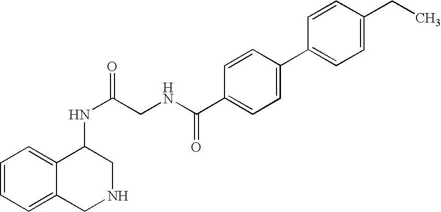 |
| 97 | N-{3-[(6-hydroxy-1,3-benzothiazol- 2-yl)amino]-3-oxo-1- phenylpropyl)benzamide | 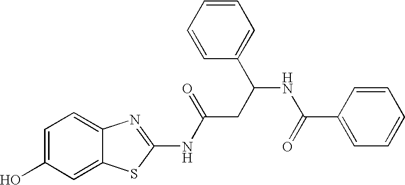 |
| 98 | N-[3-({2-[(6-hydroxy-1,3- benzothiazol-2-yl)amino]-2- oxoethyl}amino)-3-oxo-1- phenylpropyl]benzamide | 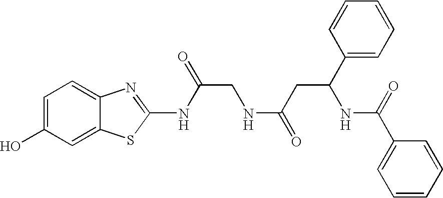 |
| 99 | 2-[(5-bromopyridin-2-yl)amino]-2- oxoethyl{[2-(naphthalen-1- ylamino)-2-oxoethyl]thio}acetate | 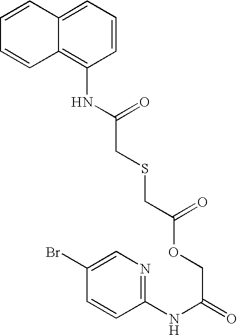 |
| 100 | 2-[(5-chloropyridin-2-yl)amino]-2- oxoethyl(1,3-benzoxazol-2- ylthio)acetate | 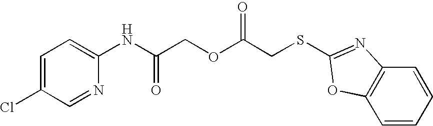 |
| 101 | 4-{[(5,6-dimethyl-4-oxothieno[2,3- d]pyrimidin-3(4H)-yl)acetyl]amino}- N-[6-(methyloxy)-1,3-benzothiazol- 2-yl]butanamide | 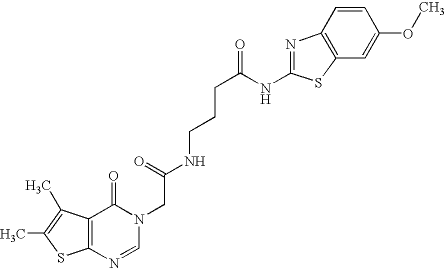 |

24. A pharmaceutical composition comprising a compound according to any one of claims 1-23 and a pharmaceutically acceptable carrier.

25. A metabolite of the compound or the pharmaceutical composition according to any one of claims 1-24.

26. A method of modulating the ill vivo activity of a kinase, the method comprising administering to a subject an effective amount of either the compound or the pharmaceutical composition according to any of claims 1-24, or a compound, or pharmaceutical composition comprising said compound and a pharmaceutically acceptable carrier, selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-dimethyl-4-oxo-4H-thieno[2,3-d]pyrimidin-3-yl)-N-[(6-methoxy-benzothiazol-2-ylcarbamoyl)-methyl]-acetamide, (naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide.

27. The method according to claim 26, wherein the kinase is at least one of KIAA1361, TAO, and JIK.

28. The method according to claim 27, wherein modulating the in vivo activity of the kinase comprises inhibition of said kinase.

29. A method of treating diseases or disorders associated with uncontrolled, abnormal, and/or unwanted cellular activities, the method comprising administering, to a mammal in need thereof, a therapeutically effective amount of either the compound or the pharmaceutical composition as described in any one of claims 1-24, or a compound, or pharmaceutical composition comprising said compound and a pharmaceutically acceptable carrier, selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-Dimethyl-4-oxo-4H-thieno[2,3-d]pyrimidin-3-yl)-N-[(6-methoxy-benzothiazol-2-ylcarbamoyl)-methyl]-acetamide, (Naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide.

30. A method of screening for a modulator of a kinase, said kinase selected from KIAA1361, TAO, and JIK, the method comprising combining either a compound according to any one of claims 1-23, or a compound selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-dimethyl-4-oxo-4H-thieno[2,3-d]pyrimidin-3-yl)-N-[(6-methoxy-benzothiazol-2-ylcarbamoyl)-methyl]-acetamide, (naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino-N-(6-methoxy-benzothiazol-2-yl)-butyramide, and at least one candidate agent and determining the effect of the candidate agent on the activity of said kinase.

31. A method of inhibiting proliferative activity in a cell, the method comprising administering an effective amount of a composition comprising a compound according any one of claims 1-23, or a compound selected from the group consisting of 3-benzoylamino-3-phenyl-propionic acid (6-methoxy-benzothiazol-2-ylcarbamoyl)-methyl ester, N-(3,4-dichloro-benzyl)-2-[4-(4-pyridin-2-yl-pyrimidin-2-yl)-phenoxy]-acetamide, 2-(5,6-dimethyl-4-oxo-4H-thieno[2,3-d]pyrimidin-3-yl)-N-[(6-methoxy-benzothiazol-2-ylcarbamoyl)-methyl]-acetamide, (naphthalen-1-ylcarbamoylmethylsulfanyl)-acetic acid (5-bromo-pyridin-2-ylcarbamoyl)-methyl ester, (benzooxazol-2-ylsulfanyl)-acetic acid (5-chloro-pyridin-2-ylcarbamoyl)-methyl ester, and 4-[2-(5,6-dimethyl-4-oxo-4H-thieno[2,3-d]pyrimidin-3-yl)-acetylamino]-N-(6-methoxy-benzothiazol-2-yl)-butyramide, to a cell or a plurality of cells.
